SEQUENCE LISTING (15 sequences)

SEQ ID NO: 1 - protein, 844 aa, unidentified
MAPTFQTALFFTIISLSFAAPNAKQVRWCAISDLEQKKCNDLVGSCNVPDITLVCVLRSSTEDCMTAIKDGQADAMFLDSGEVYEASKDPYNLKPIIAEPYSSNRDLQKCLKERQQALAKKMIGHYIPQCDEKGNYQPQQCHGSTGHCWCVNAMGEKISGTNTPPGQTRATCERHELPKCLKERQVALGGDEKVLGRFVPQCDEKGNYEPQQFHGSTGYSWCVNAIGEEIAGTKTPPGKIPATCQKHDLVTTCHYAVAMVKKSSAFQFNQLKGKRSCHSGVSKTDGWKALVTVLVEKKLLSWDGPAKESIQRAMSKFFSVSCIPGATQTNLCKQCKGEEGKNCKNSHDEPYYGNYGAFRCLKEDMGDVAFLRSTALSDEHSEVYELLCPDNTRKPLNKYKECNLGTVPAGTVVTRKISDKTEDINNFLMEAQKRQCKLFSSAHGKDLMFDDSTLQLALLSSEVDAFLYLGVKLFHAMKALTGDAHLPSKNKVRWCTINKLEKMKCDDWSAVSGGAIACTEASCPKGCVKQILKGEADAVKLEVQYMYEALMCGLLPAVEEYHNKDDFGPCKTPGSPYTDFGTLRAVALVKKSNKDINWNNIKGKKSCHTGVGDIAGWVIPVSLIRRQNDNSDIDSFFGESCAPGSDTKSNLCKLCIGDPKNSAANTKCSLSDKEAYYGNQGAFRCLVEKGDVAFVPHTVVFENTDGKNPAVWAKNLKSEDFELLCLDGSRAPVSNYKSCKLSGIPPPAIVTREESISDVVRIVANQQSLYGRKGFEKDMFQLFSSNKGNNLLFNDNTQCLITFDRQPKDIMEDYFGKPYYTTVYGASRSAMSSELISACTIKHC

SEQ ID NO: 2 - protein, 845 aa, Nanorana parkeri
MALTFHTALYFTIVGLSFAASDARHVQWCTISHLEQKKCNDLVGSCNVPDITLACVYRSSTENCMAAIKDGQADAMFLDSGDVYKASLDHYNLKPIIAEPYSLHRELTKCLKHRQESLGGDKMVKGRYIPQCDEKGNYHPVQCHASTGYCWCVNANGEKIEGTNTTPVQTPPTCPSQVLTKCLKERQEALGGKRIAIGRYIPQCDEQGNYRPMQCHGSTGYCWCVNAIGEKIEGTNTPPGNTQPTCQSHDWDTCHYAVAVVKNSSTFQFGQLKGKRSCHSGLSKTDGWNAPVNVFVEKKLLPWDGLAKGSIERAVSKFFSASCIPGATETNLCKQCIGEEEKKCKSSHDEPYYGDHGAFRCLQEDKGDVAFLKNTALPDEHSGVYELLCPDNTRKPLNKYKECNLGKVPADAVVTRKAGDKTKDINDFLLEAQKKKCKLFGSPHGKDLMFDDSTTHLAPLPSEIDAFFFLGVKWYNAMKALTEDVKLPSKNKVRWCTINKPEMMKCKDWAAVSGGAIACTEASCPEHCVKQILKGEADAVTLDVQYMYMALMCGLLPAVEEYPNKDDFHPCQIPGSTIKDFGTKRAVALVKKSNKDIKWNNLKGKKSCHTHVGDIPGWVIPAGLISNQNDNIDIESFFGESCAPGSDTNSKLCKLCIGDPENPKASTRCSLSDKEAYYGNEGAFRCLVEKGDVAFVPHTVVFANTDGKNPAEWAKDLKSEDFEILCLDGSRAPVTNYRGCNLSGLPPRAIVTREESVSDVVRILINQQSLYGRNGFEKDMFQMFSSAKGQNLLFNDETQCLIEFDRQPKDIMEDYFGVRYYTAVYSASRSAVPSELIPACTFKHC

SEQ ID NO: 3 - protein, 839 aa, Mantella aurantiaca
MALTFQTALYFTIIGLSFVTSSARDVRWCVISDLEQKKCNDLVGSCKVSGITLVCVHRSSTENCMTAIRDEQADAMFLDSGDVYKASMSPYNLKPIIAESYSSHKECLKKRQEALGGANVLGQFVPQCDEKGSYQPQQCYGSTGHCWCVNAIGEEIVGTKTQPGQTQATCERHDQTKCLKDRQKALAKKMIGGFVPQCDEKGNFKPRQCHGSTGYCWCTNANGEKIAATNTPPQQSPPTCERHDLDSCHYAVAVVKKSSIFQFNQLKGKRSCHSGVSKTDGWKAPVSVLVDKNLLSWDRAAKESIEKAVSKFFSDSCIPGATETNLCNQCMGEGGKKCMSSHDEPYYGDHGAFRCLKEDKGDVAFLKNTVLSDTHSEDYELLCPDNTRKPLNKYKECNLGKVPADAVVTRKSGDKIKDINDFLLEAQKKHCQLFSSAHGKDLMFDDSTTQLSLLPPEVDVFFFLGVQWLNTMKTLTADVKPPSKNKVRWCTVNNLEKMKCDDWSAVSGGAIECTEASSPEHCIKQIVKGEADAVTLDVQYMYEALKCGLLPAVEEYHNKDDFGPCKTPGSEIKDFGAKRAVALVKKSNKDINWHNLKGKKSCHTGVGDQAGWIIPVGLISRQIDNCNIDSFFSESCAPGSDTTSNFCKLCIGDPSNPMANTKCSLSDKEAYHGNEGAIRCLVEKGDVAFVHHTAVFENTDGKNPAEWAANLKSEDFEILCLDGSRAPVSNYMTCRLSGVPPRAIVTREESVSDVVRILINQQSLFGRNGFEKDMFQMFSSNKGQNLLFNDGTQCLIEFDRQPRGIMEDYFGKPYYNAVYNTSKCVLPSELTSACTFKHC

SEQ ID NO: 4 - protein, 912 aa, Dendrobates tinctorius
MRMTPQVLLWLCLLALSSAAPNIRNVRWCVTSDTEEQKCNDLVTSCHVDEILLTCVKKSSTEDCVRAISNGEADAISLDSKDVYKASLDPFNLKPIMTEAYSEREHTPCMRHRQSVLGGKNMKIGAFVPKCDEKGNYVPKQCHGSTGYCWCLNENGEEIEGTRTPPGNSGLTCENKANKPPCLKERQKLLSAKPSPAVFVPECDEKGNYRPQQSHVYTWCVDEYGEEVFGSRNFPGKPPKPCEASGETACIKERNKVLSVAEPLLGAFLPDCDENGYFSPLQFHGSTGYSWCVTKNGEEIKGTRTGPGQTPPTCEVSAPVTLHYAVAVVKKSSSFQLDQLKGKRSCHSAVGEAAGWVAPLNVLLKKKLLLWEEPEQKSIEKVASEIFSASCAPGAQEANLCEQCAGQEDQCTRGPGEPYYGDEGAFRCLRDGKGDVAFVEDTALTGQYSDNFELLCPDNTRRPLSQYKICNFGRIPRHSVVTRSTGDKMQDITEYLLQAQKKECKLFSSTHGKDLLFEDTTSALIALPSAMDTFLFLGPDLFNGMKTLNGARPPRVKQQIRWCPQSKNEHKKCEDWSSVSGGAIKCTEPSSALECVKKILKDEADAVNLDTAHTYTALKCGLLPSLDEYRNKDDLIPCQIPGADYTEFGSYRIVAIVKKKDKDITWNNLQGKKSCHTGVGDMMGWNIPVYLISKKTKNCDLGSYFSQSCAPGSPIDSNLCKLCIGDPQNTEANTKCSLSDKEAYYGNEGAVRCLVEKGDVAFVPHTAVFENTDGKNPALWAKDLKSADFELLCPDGSRAPVSNYKTCKLSGIANQVTVTRPESVKDVVRITQNQQSLYGRTGSLKDIFQMFSTSYGQNLLFSDRTQCLVEFDRMLDRDIMDDYFGKPFHKHLIRDNDCFPISALATACSFHH

SEQ ID NO: 5 - protein, 912 aa, Oophaga sylvatica
MRMTLQVLLCLCLLVLSSAAPNSRNVRWCVTSDTEEQKCNDLVTSCHVDEILLICVKKSSKEDCVRAISNGEADAISLDSKDVYKASLDPFNLKPIMTEAYSESEQTPCMRHRQSVLGGKKMIIGAFVPKCDEKGNYVPKQCHDSTGFCWCLNENGEEIEGTRTPPGNSGLTCENKANKPPCLKERQKLLSAKPSPAVFVPECDEKGNYRPEQSHVYSWCVDEYGEEVLGSRTFPGKPPKPCEASGETPCIKERNKVLSAAEPLPGAFVPDCDEKGYFSPLQFHGSTGHSWCVTKNGEEIKGTRTRPGQTPPTCDVPAPATLHYAVAVVKKSSSFQLDQLKGKRSCHSAVGEAAGWVAPLNVLLKKKFLLWEEPEQKSTEKVASEIFSASCAAGAQEANLCEQCAGQEDKCTRGPGEPYYGDEGAIRCLRDDKGDVAFVEDTALTGQYSDNFELLCPDNTRRPLSQYKICNFGSVPRHAVVTRSTGDKMKEITEYLLQAQKKECKLFSSTHGKDLLFEDTTSALIALPSGMDTFLFLGPDLFNGMKTLNGAHPPRVKQKIRWCPQNKNEKKKCDDWSSVSGGSIKCTEPSSALECIKMILKDEADAVNLDAEHAYTALKCGLLPSLEEYRNKDDLIVCQIPGAEYSEFGSYRIVALVKKTDKDITWNNLQGKKSCHTHVGDMIGWNIPVYLISKKTKNCDLGSYFSQSCAPGSPIDSNLCKLCIGDPQNTEANTKCSLSDKEAYYGNEGAVRCLVETGDVAFVPHTAVFENTDGKNPALWAKDLKSTDFELLCPDGSRAPVSDYKKCKLSGIANHVTVTRPESVKDVVRITQNQESLYGRTGSQKDIFQMFSSSYGQNLLFNDRTQCLFEFDRMLDRDIMDDYLGKPFHKHLMSDNDCLPKSALATACSFHH

SEQ ID NO: 6 - protein, 923 aa, Ranitomeya imitator
MRRSSYAASQEMRMTLRVLLCLCLWALSSAAPNTRNVRWCVTSDTEEQKCNDLVTSCHVDEILLICVKKSSTEDCVRAISNGEADAISLDSKDVYKASLDPFNLKPIMTEAYSESEHTPCMRHRQSVLGGKIMKIGAFVPKCDEKGNYVPKQCHGSTGYCWCLNENGEEIEGTRTPPGNPGLTCENKANKPPCLKERQKLLSAKPSPAVFVPECDEKGNYRPQQSHVYSWCVDEYGEEVFGSRSFPGKPPKPCEASGETPCIKERNKVLSAAEPLLGAFVPDCDEKGYFSPLQSHGSTGYSWCVTKNGDEIKGTRTGPGQSPPTCEVPAPVTLHYAVAVVKKSSSFHLDQLKGKRSCHSAVGEAAGWVAPLNVLLKKKLLLWEEPEPKTIEKVASEIFSASCAPGAQEANLCEQCAGQEDKCTRGPGEPYYGDEGAFRCLREDKGDVAFVEDTALTGQYSDNFELLCPDNTRRPLSQYKICNFGRIPRHAVVTRSSGDKMKDITEYLLQAQKKECKLFSSTHGKDLLFEDTTSALIALPSAMDTFLFLGPDLFNGMKTLNGARPPQVNQQIRWCPQSKNEKKKCDDWSSVSGGAIKCTEPSSGLECIKMILKGEAEAVNLDIEHSYIALKCGLLPSVDEYRNKDDLIPCQIPGSEYSEFGSYRIVALVKKTDKDLTWNNLQGKKSCHTRAGDMIGWNIPVYLISKKTKNCDLGSYFSQSCAPGSPIDSNLCKLCIGDPQNPQANTKCSISDKEAYYGHEGAIRCLVEKGDVAFVPHTAIFENTDGKNPALWAKDLKSTDFELLCPDGSRAPVNDYKKCKLSGIANQIAITRPESVKDVVRIIQNQQSLYGRTGSQKDIFQMFSSSYGQNLLFSDRTQCLVEFDRMLDRDIMDDYFGKPFHKHLLRDNDCLPKSALASACSFHH

SEQ ID NO: 7 - protein, 914 aa, Phyllobates terribilis
MRMTTAFQVLLCVCLLALSSAAPNARNVRWCVTSDAEEQKCNDLVTSCHVDEILLTCVKKYSTEDCVRAISNGEADAISLDSKDVYKASLHPFNLKPIVTEAYSEKEHTPCMRHRQSVLGGKKIKIGAFVPKCDEKGNYVPKQCHGSTGYCWCLNENGEEIEGTRTPPGTKHLTCEDTADKPPCLKKRQKLLSAKPSPAVFVPECDEKGNYRPEQSHVYTWCVDEYGEEVFGSRNFPGKPPKPCVASDEPLCIKERNKVLGTTEPLLGAFVPDCDEKGYFSPLQFHGSTGYSWCVTENGVEIKGTRTGPGQTPPTCEVPAPMTLNYAVAVVKKSSSFHLDQLKGKRSCHSAVGEAAGWVAPLNVLLKKKLLSWGGPEQKSIEKVASEIFSASCAPGAQEANLCEQCAGQEDKCTRGPGEPYYGDEGAFRCLRDDKGDVAFVEDTVLSGQYSDNFELLCPDNTRRPLSQYKICNFGRIPRHAVVTRTTGDKMKDITEYLVQGQKKECKLFSSTHGKDLLFEDTTTALITLPSAMDTFFFLGSELFKGMKTLEGTRPPRVHREIRWCPQNKNEKKKCDDWSSVSGGAIKCTEPSSALECIEMILKGEAEAVNLDAEHAYTALKCGLLPSVEEYRNKDDLIPCQVPGAEYSEFGGNRIVALVKKTDKDITWNDIQGKKSCHTHVGDMIGWNIPIYLIYKKTKNCDIGSFFSQSCAPGSTIDSNLCKLCIGDPQNPEANTKCSLSDKEAYYGNEGAIRCLVEKGDVAFVPHTAVYENTDGKNPALWAKDLKSTDFELLCLDGSRAPVSDYKKCKLSGIVNQVTVTRPESVKDVVRIIHNQESLYGRTGFQKDIFQMFSSSYGENLLFSDRTQCLFEFDRMLDRDIMEDYLGKPFHKYVMRDNECLPKSALATACSFHH

SEQ ID NO: 8 - protein, 919 aa, Epipedobates tricolor
MVSSRKLRMTSAFQLLLCLCLMVLSFAAPNARNVRWCVTSDAEEHKCNDLVNSCHVNDILLKCVKKSSTEDCVRAISNGEADTISLDSKDVYKASLHPFNLKPIMTEGYSEREHTPCMRHRQSVLEGKKMKIGAFVPKCDEKGNYAPKQCHGSSGYCWCLNENGEEIEGTRTPPGTKSLTCEDAANKPPCLKERQKLLSSKPAPTVFVPECDEKGNYRPQQCHIYCWCVDEYGEEVLFSRSFPGKPPKKCEPSGETLCLKQRNKVLSATEPLLGTFVPECDDKGYFTPQQFHGSTGYSWCVTKNGDEIPGTRTGPGQTPHICEVKAPVTLHYAVAVVKKSSSFQLDELKGKRSCHSAVGEAAGWVAPLKILLKKKLLSLEGAEVKSIEKAASEIFSASCAPGAHEAKLCEQCAGQEGKCHRGPGEPYYGDEGAFRCLRDNKGDVAFVEDTALSGQYSDNYELLCPDNTRRPLSQYKICNFGRVPRHAVVTRSSGDKMKDITEYLLEAQKKECKLFSSTHGKDLLFEDTTTGLIALPSAMDTFLFLGPELFHGMKTLDGAHPPSSQEIRWCPLTKNEKKKCDDWSSVSGGAIKCTEPSSGQKCIEKILKGEADVVTLGVEHAYTALKCGLHISFEEYRNKDDLIPCQVLEAEYTEFGSYRIVAVVKKTDKDITWNNLQGKKSCHTSVNDMVGWKIPHALLYKKTKSCDFGSYFSKSCAPGSAIDSNLCKLCIGDPQNTKSNTKCSLSDKEAYYGNDGAIRCLVEKGDVAFVPHTAIFENTDGKNPALWAKDLKSTDFELLCLDGSRAPVSDYKTCKLSGIANQVTVNRPERQADVLRIILNQQSLYGRTGSQKDIFQMFSSSYGQNLLFNDRTQCLFVFDRMLDRDIVDDYIGKAMQRDVLRDNECFPQTALTNACLFHH

SEQ ID NO: 9 - protein, 913 aa, Allobates femoralis
MRMTPALLGLLCLCLLVLSSAAPNSRTVRWCVTSDTEQQKCNDLVNSCHVDEIVLSCVKKSNTEDCVRAISTGEADAISLDSKDVYKASLDPFHLKPIMTEGYSERDHTPCMKHRQSVLGGKQLIIGAFVPKCDEKGNYAPKQCHGSSGYCWCLNENGEEIKGTRSRPGTKVLTCEDAANKPPCLKERQKLLSENPSPTVFVPECDEKGNYRPRQCHDYCWCVDEYGEEIFGSRTFPGKPPKACEASGETLCIKERNKVLSTAEPLRGAFLPECDEKGYFSPMQCHSSTGHCWCTTKDGEEIEGTRTGPGQSRPTCDIPAPGTVHYAVAVVKKSSNFQLDGLKGKRSCHSAVGESGGWVAPLNIFLKKNLLSWEGPEQKSIEKAASEFFSASCAPGAQESTLCEQCAGQEDKCKRGPGEPYYGDEGAIKCLKDDKGDVAFVEDTILSGQYSNDFELLCSDNTRRPLSQYKMCNFGKIPRHAVVTRSSGDKMEDITEYLLKAQKKECKLFGSAVGKDLLFEDTTTSLIVLPSAMDTFLYLGPELFSGMKTLEGAHPPSNQEIRWCPQSKNEKKKCDDWSSVSGGAIKCTEPSSALECIEKILKGEAEAVTLDGEHAYTALKCGLVPSLDEYRNKDDLVPCQIPGAEYSEFGSYRIVALVKKTDKDITWDNLQGKKSCHTSVGDMMGWTVPISLIYKKTKNCDIGSYFSQSCAPGSAIDSNLCELCIGDPGNPTANTKCSQNDKEAYYGNEGAIRCLVEKGDVTFVPHTAVYENTDGKNPALWAKDLKSTDFELLCPDGSRAPVSDYKRCKLSGIANQITVTRSESLKDVVRIILNQQSLYGRNGFQKDIFQLFSSVHGQNLLFSDRTQCLVEFDRMLDRDIMDDYFGKPFHKTVFRDNDCLPKSALATACSFHH

SEQ ID NO: 10 - protein, 944 aa, unidentified
MTPAFQVLLCLCLLGMSYAAPQARNVRWCVTSDAEEEKCNDLVNTCTVKEILLICVKKSSTEDCIRAISAGEADAISLDSEDIYQASLKPFHLKPIMTETYASKKNIKLNQSPCQKEHQHLLEGGPKCDETESTPCLRHRQRVLGARKPQIGEFVPECDEKGNYFPKQCYGTTGYCWCVDEHGDEIPVGRAKQGKVNISCEYAASEKPCMKEQRKALSGGQPLRGAFIPNCDEKGNYSPKQCHGSTGYCWCVDENGAEISGSRTPPGQQVPTCGSYVGATCIKDRYKVLGAGKPLPGAFVPDCDEKGDYRPQQCHGSTGHCWCVSKDGVEIQGTRAAPGQSPPTCEDPDLVKSHYAVAVVKKSSTLQFNHLKGKRSCHSAVGKTAGWIAPLYALYKKHFLLWDGSEEKSFEQAASEFFSASCAPGAKEENLCKQCAGQEDNCKSSPGEPYYGDEGALRCLREDKGDVAFVEHTALSGKDSDNFELLCPDNTRSPVSEHKDCNFGKVPRHAVVTRDTGDKSKDIIEYLLEAQKKECKLFSSTCGKDLMSGDTTTNLIALPSGMDAFLFLGPELFNAMKTLHGAPLPSKNEILWCTQNKNEKTKCDDWSAVSGGAIKCTEPLSADQCIEKILKREADAVTLHTEHMYTALKCGLVPAVEGYYNKDDFTPCKTDGDFYKDFGGTYVVALVKKSDKDITWNNLKGKKSCHTGVGHWTGWMIPVSQISKQTKNCDIGSYFSQSCAPGSAIDSNLCELCIGDQQNTKAQTQCSPSDKEAYYGGAGAIRCLVEKGDVAFVTDRDVFKNTDGNNPALWAKDLKSTDFELLCLDGSRAPVTDYKNCNLPGNPNLSVVSREENVNDVVRIFLNQQSLYGRNGSQKDKFQMFSSYGENLLFSDDAQCMIEFDMIKGRDLIDDVLGKHFSATMLRNNPCLPKSELRTACTFRAEKI

SEQ ID NO: 11 - protein, 1954 aa, Bufo bufo
MKHSSSSAQKEVRMTPAFQVLLCLCLLGMSYAAPQARNVRWCVTSDAEEEKCNDLVNSCPVKEILLICVKKSSIEDCMRAISAGEADAISLDSENIYKASLKPFHLKPIMTETYISKKDIKLTQGPCQKEHQHLLEGRQHKCEETESTPCLRHRQRVLGAKKPHIGEFMPECDEKGNYFPKQCYGTTGYCWCVDENGAERHVGRAKQGKVNISCEYTESQKPCMKERRKVLSGGQPLPGAFIPDCDEEGNYNPKQCHGSTGYCWCVDENGAEISGSRMPTGQSDPTCGTYAGATCIKDRYKVLGAGEALRGAFVPDCDEKGDYRSKQCHGSTGYCWCVSKYGVEIQGTRTAPGQSPTTCEIPGGEKPCMKERRKSLSGGQPLPGAFMPDCDEKGNYSPKQCHGSTGYCWCVNENGKEISGSRTPPGQQVPTCVASVLSAETSCIKERQKVLGAVKPILGAFVPDCDEKGEYRPKQCHGSTGYCWCVSKDGKEIQGTRAAPGQSPPTCQDTGEKLVLSAETSCIKERQKVLGAVKPILGAFVPDCDEKGEYRPKQCHGSTGYCWCVSKDGKEIQGTRAARGQSPPTCQDTGEKLVLSAETSCIKELQKVLGAVKPMVGAFVPDCDEKGEYRPKQCHGSTGYCWCVSKDGKEIQGTRVAPGQSPPTCQDTVLSAETSCIKERQKVLGAVKPILGAFLPDCDEKGEYRPKQCHGSTGYCWCVSKDGKEIQGTRAAPGQSPPTCQDTGEKLVLSAETSCIKERQKVLGAVKPIVGAFVPDCDEKGEYRPKQCHGSTGHCWCVSKDGKEIQGTRVAPGQSPPTCQDTVLSAETSCIKERQKVLGAVKPILGAFLPDCDEKGEYRPKQCHGSTGYCWCVSKDGKEIQGTRAAPGQSPPTCQDTVLSAETSCIKERQKVLGAVKPILGAFLPDCDEKGEYRPKQCHGSTGYCWCVSKDGKEIQGTRAAPGQSPPTCQDTGEKLVLSAETSCIKERQKVLGAVKPIVGAFVPDCDEKGEYRPKQCHGSTGHCWCVSKDGKEIQGTRVAPGQSPPTCQDTVLSAETSCIKERQKVLGAVKPILGAFLPDCDEKGEYRPKQCHGSTGYCWCVSKDGKEIQGTRAAPGQSPPTCQDTGEKLVLSAETSCIKERQKVLGAVKPIVGAFVPDCDEKGEYRPKQCHGSTGHCWCVSKDGKEIQGTRVAPGQSPPTCQDTVLSAETSCIKERQKVLGAVKPILGAFVPDCDEKGEYRPKQCHGSTGYCWCVSKDGKEIQGTRAAPGQSPPTCQDTGEKLVLSAETSCIKERQKVLGAVKPIVGAFVPDCDEKGEYRPKQCHGSTGHCWCVSKDGKEIQGTRVAPGQSPPTCQDTDPVTCHYAVAVVKKSSTLQFNQLKGKRSCHSAVGKTAGWIAPLYKLYKKNLLLWEKPEEKSFEKAASEFFSVSCAPGAKEENLCKQCAGKEDKCKRSPGELYYGDEGALRCLREDKGDVAFLEDIALSGQDLDNFELLCPDNTKSPLSEQKHCHFGKVPSHAVVTRSTGDKSKDIIEYLLEAQKKGCKLFSSTHGKDLMFEHTTTNVIALPSAMNTFLFLGPELFDAMKTLHGTPLPSKNEVRWCTQNQNEKTKCDDWSSVSGGAIKCTEPSSVQQCIEKILKHEADAVTLSAEHMYTALKCGLVPAVDEYHNKDDFAPCRTLGDIYTDFGTPRAVALVKKSNKDITWNNLKGKKSCHTGVGHMAGWVIPLSLISKQTNNCDLGSYFSQSCAPGSAIDSNLCKLCIGDPQNTKAQTQCSPSDKEAYYDSAGAIRCLVEKGDVAFLPHTAVFENTDGNNPALWAKDLKSTDFELLCPDGSRAPVTDYKNCKLLSISSPSVVTREESVSDVVRIVLSQQSLFGRKGFEKDMFQMFSSSNGKNLLFSDGTQCLLEFDRIIGRDIMEDYFGKPFHTAVHRDNQCLPTSELASACAFHHC

SEQ ID NO: 12 - protein, 1821 aa, Bufo gargarizans
MKHSSSSAQKEVRMTPAFQVLLCLCLLGMSYAAPQARNVRWCVTSNAEEKKCNDLVNSCTVKEIVLICVKKSSTEDCMRAISAGEADAISLDSENIYKASLNPFDLKPIMTETYPSQKEIKVNQGPCQKERQRQRERGRPLLGAFEPKCDEKGNYQPKQCHGSTGYCWCVNEEGKTIDGTKTPPGQKSVTCEDHQQSTPCLRHRQSVLGANKPQIGAFVPDCDEKGNYSPKQCFGSTGYCWCVDEHGDEIEGVRAKQGKVNITCEYTGGEKPCMKERRKSLSGGQPLPGAFMPDCDEKGNYSPKQCHGSTGYCWCVNENGKEISGSRTPPGQQVPTCGASVLSAETSCIKERQKVLGAATPILGAFVPDCDAKGDYRPKQCHGSTGHCWCVSKDGKEIQGTRTAPGQTPPTCEIPGGEKPCMKERRKSLSGGQPLPGAFMPDCDEKGNYSPKQCHGSTGYCWCVNENGKEISGSRTPPGQQVPTCGASVLSAETSCIKERQKVLGAEKPILGAFVPDCDEKGDYRPKQCHSSTGHCWCVSKDGKEIQGTRTAPGQTPPTCEIPGGEKPCMKERQKSLSGGQPLPGAFMPDCDEKGNYSPKQCHGSTGYCWCVNENGKEISGSRTPPGQQVPTCGASVLSADTSCIKERQKVLGAEKPILGAFEPDCDEKGDYRPKQCHGSTGHCWCVSKDGKEIQGTRTARGQSPPTCEIPVLSAETSCIKERQKVLGAATPILGAFVPDCDEKGDYRPKQCHGSTGHCWCVSKDGKEIQGTRAAPGQTPPTCEDRVLSAETSCIKERQKVLGAATPILGAFVPDCDEKGEYRPKQCHGSTGHCWCVSKDGKEIQGTRAGPGQTPPTCEDTAETSCIKERQKVLGAAKPILGAFVPDCDEKGEYRPKQCHGSTGHCWCVSKDGKEIQGTRAAPRQSPPTCEIPVLSAETSCIKEQQKVRAGKPILGAFVPDCDEKGDYRPKQCHGSTGHCWCVSKDGKEIQGTRAACGQSPPTCEIPAETSCIKERQKVLGAEKPILGAFVPDCDEKGEYRPKQCHGSTGHCWCVSKDGKEIQGTRAGPGQSPPTCEDTVLSAETSCIKERQKVLGAATPILGAFVPDCDEKGDYRPKQCHGSTGHCWCVSKDGKEIQGTRAGPGQSPPTCEIPAEKSCIKERQKVRSPRKPILGAFVPDCDEKGDYRPKQCHSSTGHCWCVSKDGKEIQGTRAARGQSPPTCEDPDPVTCHYAVAVVKKSSTLQFNQLKGKRSCHSAVGKTASWIAPLYKLYKKNLLLWEKPEEKSFEKAASEFFSVSCAPGAKEENLCKQCAGKEEKCKRSPGEPYYGDEGALRCLRDDKGDVAFVEHTALSGQDLDNFELLCPDNTRSPLSEHKHCHFGKVPRHAVVTKSTGDKSKDIIEYLLEAQKKGCKLFSSTHGKDLIFEHTTKNLIALPSAMNTFLFLGPELFDAMKTLHGAPLPSKNEVRWCTQNKKEKTKCDDWSSVSGGAIKCTEPSSVQQCIEKILKHEADAVTLSAEHMYTALKCGLVPAIDEYHNKDDLAPCKTLGDIYTDFGTPRAVALVKKSDKGITWNNLKGKKSCHTGVGHMAGWVIPLSLISKQTNNCDMGSYFSQSCAPGSAIDSNLCKLCIGDPQNTKAQTKCSPSDKEAYFGSAGAIRCLVEKGDVAFLPHTAVIENTDGNNPALWAKDLKSTDFELLCPDGSRAPVTDYKNCKLLSISSPSVVTREESVSDVVRIVLNQQSLFGQKGFEKDIFQMFSSSNGKNLLFSDGTQCLLEFDRIIGRDIMEDYFGKRFHTAVHRDNQCLPTSEFASACAFHHC

SEQ ID NO: 13 - protein, 764 aa, Engystomops pustulosus
MTAISHGEADAISLHGENIYKASFKPFNLIPIITESYHYQKGGRPLLGAFVPKCDEKGNYSPTQCHGGTGYCWCVTAEGKEIDGTKKPPGESPTCGEETKKTPCLNKREKALSGGQPLLGAFAPDCDEEGNFKPRQCYGSTGYCWCVDDNGVEISATRTPPGEKPPTCGATDPVTCHYAVAVVKKSSTFQFNQLKGKRSCHSAVGESAGWVAPMSALLDKKFLLWEGPAKKSFEKAASEFFSASCAPGAKEENLCKQCAGQQDKCKQSPGEPYYGDEGAFRCLREDKGDVAFVEHTVLSGQYSDNYELLCPDKTRKPLSQYEHCHFGKVPRPAVVTRHTGRKTKDITDYLLEAQKKECKLFSSTHGKNPPDTTTTLTSLPLAMDTFLFLGPELFNAMKRMHGEPLPSNKEVRWCPQSSDEKKKCDDWSAVSGGAIKCTEPFTTLQCIEKVLKGEAEAVTLNVENMYTALKCGLVPIVEEYHNKDDFNPCQSRGAQIKDFGTVKAVAVVKKRDKDINWNNLKGKKSCHTGVGHLAGWVVPVSLINKQSRTCDVESYFNQSCAPGSTTKSNLCKLCIGDPQNTKAKTKCSPNDKEAYYGNEGAIRCLAEKGDVAFVPHTAVFENTDGKNPALWAKDLKSTDFELLCPDGSRAPVSNYKNCKLAGIPPRAVVTRKDSASDVTKIIVNQQSLFGRKGFQKDIFQMFSSSNGQNLLFSDNTQCLIEFDRMIEKDIMEDYFGKTYYTAVHSDNQCFPPSALASACSFHHC

SEQ ID NO: 14 - protein, 852 aa, Eleutherodactylus coqui
MTPAFQLLLCLCLLGLSYTAPQGRNVRWCVTSDIEDKKCNDLANSCTVNEIHLKCVKKSSTLDCVKTISDGEADAISLDGEDIYKASLNPFNLKPIMVESHYHRAKNLPCLKERQEALGGGHPLPGAFVPNCDEKGNYNPKQCHGSTGYCWCVNKNGQKINGTETPPGQPSPTCEEEKSTPCLKHRQRLLGGNKPKIGAFVPKCDEKGDYIPKQCHGSTGQCWCVNTDGEEIAGTRTGPGKAPPTCEDAEPVTCHYAVAVVKKSSTLQFDQLKGKRSCHSAVGTAAGWAAPLNALMKRNLLVWKGPKEKTIEKAASEFFLSSCAPGAKETNLCKQCAGKEANCKHSSEEPYYGDEGAFKCLRDDKGDVAFVENSVLSEEHTDSFELLCPDNTRKQLSQYKDCNFGKVPRHAVVTRSSGDKIKDITEYLLGAQKKECELFSSTHGKRFLFEDTTTNLIALPSAMDTFLFLGPELFDAMKALHGEHPPSKNEVRWCTLSEKEKLKCDDWSSVSGGAIKCTEPSLAEECIEKVLKGEADVVSLSKKHMYAALTCGLIPAFEEYHNKDDFGPCQTPGTQYTDFGMTSNVALVKKKDQDITWNNLKGKKSCHPEVDDSAGWVDPLILIKKQTKSCDLGSFFSKSCAPGSAIDSNLCELCIGDSQNSKANTKCSRSDKEAYYGNDGAIRQKGDVAFVPHFAVSDNTDGKNPALWAKDLKSSDFELLCPDGSRAPVSDHKKCKLGGASIQTVVCREDRVSDVVRIIQNQQSLYGRKGFQKDIFQIFSSRHGRNILFHDDTQCLVDFDRVKEIDIMYDFFGKEHYDSIDFCLLIPSLIRRLGNRDLVIEMSLLVVFIPPS

SEQ ID NO: 15 - protein, 844 aa, Rana temporaria
MAPTFKAALFFTIISLSFAAPNAKQVRWCAISDLEQKKCNDLVGSCNVQDITLVCVLRTSTEDCLTAIKDGQADAMFLDSGDVYEASKDPYNLKPIIAEPYSSHKDLQKCLKEREQALAKKMIGHHIPQCDEKGNYQPQQCHGSTGQCWCVNGMGEKISGTNTPPGQTRATCERHELPKCLKERQVALGGDEIVLGRFVPQCDEKGNYEPQQFHGSTGYSWCVNEIGEEIAGTKTPPGKIPVTCEKHDLVTTCHYAVAMVKKSSTFQFNQLKGKRSCHSGVSKTDGWKALVTVLVEKKLLSWDGPAKESIQQAMSKFFSVSCIPGATQTNLCKQCKGEEGKNCKNSHDEPYYGNHGASRCLKEDKGDVAFLRSTALSDEDSEAYELLCPDNTRKPLNKYKECNLGTVPVGAVVTRKIGDKTVDINNFLMEAQKKQCKLFSSAHGKDLMFDDSTLQLALLSSGVDAFLYLGVKLFNTMKALTEDAHLSSKNKVRWCTINKLEKMKCDDWSAVSGGAIECTEASCPKCCIKQILKGEADAVKLEVQYMYEALTCGLLPAVEEYHNKDDFGPCKTPGSPYTDFGSPRAVALVKKSNKDINWNNIKGKKACLTGVGDIAGWVIPVSLIRRQNDNCDIDSFFGESCAPGSDTKSNLCKLCIGDPKNSVANTKCSLGDNEAYHGNKGAFRCLVEKGDVAFVPHTVVFENTDGKNPAEWAKKLKSEDFELLCLDGSRSPVSNYKSCKLSGIPPPAIVTREQSVTDVVRIVTNQQSLYGRKGFEKDIFQLFSSNKGKNLLFNDNTQCLIEFDRQPKDIMEDYFGKSYYTTVYGASRSAMSSELISACTVKHC

(19) United States
(12) Patent Application Publication
(10) Pub. No.: US 2026/0063625 A1
(43) Pub. Date: Mar. 5, 2026

(54) COMPOSITIONS FOR AND METHODS OF DETECTING FOOD TOXINS

(71) Applicants: THE REGENTS OF THE UNIVERSITY OF CALIFORNIA, Oakland, CA (US); THE BOARD OF TRUSTEES OF THE LELAND STANFORD JUNIOR UNIVERSITY, Stanford, CA (US)

(72) Inventors: Daniel L. MINOR, Oakland, CA (US); Justin DU BOIS, Stanford, CA (US); Lauren O'CONNELL, Stanford, CA (US); Zhou CHEN, Oakland, CA (US); Sandra ZAKRZEWSKA, Oakland, CA (US); Fayal ABDEREMANE-ALI, Oakland, CA (US); Aurora ALVAREZ-BUYLLA, Stanford, CA (US)

(73) Assignees: THE RGENTS OF THE UNIVERSITY OF CALIFORNIA, Oakland, CA (US); THE BOARD OF TRUSTEES OF THE LELAND STANFORD JUNIOR UNIVERSITY, Stanford, CA (US)

(21) Appl. No.: 18/873,224

(22) PCT Filed: Jun. 9, 2023
(86) PCT No.: PCT/US2023/068216

§ 371 Date: Dec. 9, 2024

Related U.S. Application Data
(60) Provisional application No. 63/350,806, filed on Jun. 9, 2022.

(51) Int. Cl. G01N 33/53; G01N 33/543

(52) U.S. Cl. CPC G01N 33/5308; G01N 33/54366; G01N 2333/43504

(57) ABSTRACT

The present disclosure is directed, in part, to saxiphilin proteins, nucleic acids encoding the same, compositions comprising the same, kits comprising the same, and methods of detecting toxin in samples. In some embodiments, the toxin is saxitoxin or derivatives of the same.

### CROSS-REFERENCE TO RELATED APPLICATIONS

This application is a 35 U.S.C. § 371 national stage of and claims priority to international application PCT/US2023/068216, which was filed Jun. 9, 2023, entitled, “COMPOSITIONS FOR AND METHODS OF DETECTING FOOD TOXINS,” and claims priority to U.S. Provisional Application Ser. No. 63/350,806, which was filed Jun. 9, 2022, in accordance with 35 U.S.C. § 119 (e), both of which are incorporated by reference in their entireties.

### STATEMENT REGARDING FEDERALLY SPONSORED RESEARCH

This disclosure was made with government support under grant numbers HDTRA11910040 and HDTRA12110011 awarded by the United States Department of Defense. The United States government has certain rights in this invention.

### FIELD OF THE DISCLOSURE

The present disclosure is directed, in part, to saxiphilin proteins, nucleic acids encoding the same, compositions comprising the same, kits comprising the same, and methods of detecting toxin in samples. In some embodiments, the toxin is saxitoxin or derivatives of the same.

### BACKGROUND

Saxitoxin (STX), one of the most lethal non-peptidyl neurotoxins, blocks the bioelectrical signals in nerve and muscle required for life by inhibiting select voltage-gated sodium channel (Na_{V}) isoforms (Thottumkara et al., Hille, Llewellyn). Cyanobacteria and dinoflagellate species associated with oceanic red tides produce this bis-guanidinium small molecule and its congeners whose accumulation in seafood can cause paralytic shellfish poisoning (PSP), a commercial fishing and public health hazard of growing importance due to climate change (Thottumkara et al., Llewellyn, Wiewse et al., Anderson et al.). Its extreme lethality has also earned STX the unusual distinction of being the only marine toxin declared a chemical weapon (Thottumkara et al., Llewellyn). Select vertebrates, particularly frogs, resist STX poisoning (Prinzmetal et al., Kao et al., Mahar et al., Abderemane-Ali et al.), a property that is thought to rely on the ability of the soluble ‘toxin sponge’ protein saxiphilin (Sxph) to sequester STX (Mahar et al., Abderemane-Ali et al.).

### SUMMARY OF EMBODIMENTS

The present disclosure relates to compositions and kits comprising analogs and functional fragments of a saxiphilin amino acid sequence. In some embodiments, the amino acid sequences of the disclosure relate to an American Bullfrog saxiphilin sequence (SEQ ID NO:1), or functional fragments and variants thereof. SEQ ID NO:1:

MAPTFQTALFFTIISLSFAAPNAKQVRWCAISDLEQKKCNDLVGSC
NVPDITLVCVLRSSTEDCMTAIKDGQADAMFLDSGEVYEASKDPY
NLKPIIAEPYSSNRDLQKCLKERQQALAKKMIGHYIPQCDEKGNY
QPQQCHGSTGHCWCVNAMGEKISGTNTPPGQTRATCERHELPKCL
KERQVALGGDEKVLGRFVPQCDEKGNYEPQQFHGSTGYSWCVNAI
GEEIAGTKTPPGKIPATCQKHDLVTTCHYAVAMVKKSSAFQFNQL
KGKRSCHSGVSKTDGWKALVTVLVEKKLLSWDGPAKESIQRAMSK
FFSVSCIPGATQTNLCKQCKGEEGKNCKNSHDEPYYGNYGAFRCL
KEDMGDVAFLRSTALSDEHSEVYELLCPDNTRKPLNKYKECNLGT
VPAGTVVTRKISDKTEDINNFLMEAQKRQCKLFSSAHGKDLMFDD
STLQLALLSSEVDAFLYLGVKLFHAMKALTGDAHLPSKNKVRWCT
INKLEKMKCDDWSAVSGGAIACTEASCPKGCVKQILKGEADAVKL
EVQYMYEALMCGLLPAVEEYHNKDDFGPCKTPGSPYTDFGTLRAV
ALVKKSNKDINWNNIKGKKSCHTGVGDIAGWVIPVSLIRRQNDNS
DIDSFFGESCAPGSDTKSNLCKLCIGDPKNSAANTKCSLSDKEAY
YGNQGAFRCLVEKGDVAFVPHTVVFENTDGKNPAVWAKNLKSEDF
ELLCLDGSRAPVSNYKSCKLSGIPPPAIVTREESISDVVRIVANQ
QSLYGRKGFEKDMFQLFSSNKGNNLLFNDNTQCLITFDRQPKDIM
EDYFGKPYYTTVYGASRSAMSSELISACTIKHC

The present disclosure provides proteins or amino acid sequences comprising at least 80% sequence identity to SEQ ID NO:1 and comprising at least one of the following amino acid substitutions in SEQ ID NO:1: an alanine for the isoleucine at position 782; an alanine for the tyrosine at position 558; an isoleucine for the tyrosine at position 558; an asparagine for the aspartic acid at position 785; an alanine for the lysine at position 789; an alanine for the threonine at position 563; a phenylalanine for a tyrosine at position 558; a glutamic acid for the glutamine at position 787; an alanine for a tyrosine at position 795; an alanine for the glutamine at position 787; a tyrosine for the phenylalanine at position 784; a phenylalanine for the isoleucine at position 782; an alanine for the phenylalanine at position 561; an alanine for the aspartic acid at position 785; a leucine for the phenylalanine at position 784; an alanine for the proline at position 727; an aspartic acid for the glutamic acid at position 540; an alanine for the phenylalanine at position 784; a glutamic acid for the aspartic acid at position 794; a cysteine for the phenylalanine at position 784; a asparagine for an aspartic acid at position 794; a serine for the phenylalanine at position 784; a glutamine for the glutamic acid at position 540; an alanine for the aspartic acid at position 794; an alanine for the glutamic acid at position 540.

The present disclosure provides proteins or amino acid sequences comprising at least 80% sequence identity to SEQ ID NO:1 and comprises a tyrosine for the isoleucine at position 559.

In some embodiments, the protein comprises at least 80% sequence identity to SEQ ID NO:1, and comprises at least one of the amino acid substitutions in SEQ ID NO:1. In some embodiments, the protein comprises at least 90% sequence identity to SEQ ID NO:1, and comprises at least one of the amino acid substitutions in SEQ ID NO:1. In some embodiments, the protein comprises at least 95% sequence identity to SEQ ID NO:1, and comprises at least one of the amino acid substitutions in SEQ ID NO:1. In some embodiments, the protein comprises at least 99% sequence identity to SEQ ID NO:1, and comprises at least one of the amino acid substitutions in SEQ ID NO:1. In some embodiments, the protein comprises at least 80% sequence identity to SEQ ID NO:2, and comprises at least one of the amino acid substitutions in SEQ ID NO:2. In some embodiments, the protein comprises at least 90% sequence identity to SEQ ID NO:2, and comprises at least one of the amino acid substitutions in SEQ ID NO:2. In some embodiments, the protein comprises at least 95% sequence identity to SEQ ID NO:2, and comprises at least one of the amino acid substitutions in SEQ ID NO:2. In some embodiments, the protein comprises at least 99% sequence identity to SEQ ID NO:2, and comprises at least one of the amino acid substitutions in SEQ ID NO:2.

In some embodiments, the protein comprises at least 80% sequence identity to SEQ ID NO:1, and comprises more than one of the amino acid substitutions in SEQ ID NO:1. In some embodiments, the protein comprises at least 90% sequence identity to SEQ ID NO: 1, and comprises more than one of the amino acid substitutions in SEQ ID NO:1. In some embodiments, the protein comprises at least 95% sequence identity to SEQ ID NO:1, and comprises more than one of the amino acid substitutions in SEQ ID NO:1. In some embodiments, the protein comprises at least 96% sequence identity to SEQ ID NO:1, and comprises more than one of the amino acid substitutions in SEQ ID NO:1. In some embodiments, the protein comprises at least 97% sequence identity to SEQ ID NO:1, and comprises more than one of the amino acid substitutions in SEQ ID NO:1. In some embodiments, the protein comprises at least 98% sequence identity to SEQ ID NO:1, and comprises more than one of the amino acid substitutions in SEQ ID NO:1. In some embodiments, the protein comprises at least 99% sequence identity to SEQ ID NO:1, and comprises more than one of the amino acid substitutions in SEQ ID NO:1.

In some embodiments, the protein or amino acid sequence comprises at least 80% sequence identity to SEQ ID NO:2, and comprises more than one of the amino acid substitutions in SEQ ID NO:2. In some embodiments, the protein comprises at least 90% sequence identity to SEQ ID NO:2, and comprises more than one of the amino acid substitutions in SEQ ID NO:2. In some embodiments, the protein comprises at least 95% sequence identity to SEQ ID NO:2, and comprises more than one of the amino acid substitutions in SEQ ID NO:2. In some embodiments, the protein comprises at least 96% sequence identity to SEQ ID NO:2, and comprises more than one of the amino acid substitutions in SEQ ID NO:2. In some embodiments, the protein comprises at least 97% sequence identity to SEQ ID NO:2, and comprises more than one of the amino acid substitutions in SEQ ID NO:2. In some embodiments, the protein comprises at least 98% sequence identity to SEQ ID NO:2, and comprises more than one of the amino acid substitutions in SEQ ID NO:2. In some embodiments, the protein comprises at least 99% sequence identity to SEQ ID NO:2, and comprises more than one of the amino acid substitutions in SEQ ID NO:2.

In some embodiments, the protein or amino acid sequence comprises SEQ ID NO:1, SEQ ID NO:2, SEQ ID NO:3, SEQ ID NO:4, SEQ ID NO:5, SEQ ID NO:6, SEQ ID NO:7, SEQ ID NO:9, SEQ ID NO:10, SEQ ID NO:11, SEQ ID NO:12, SEQ ID NO:13, SEQ ID NO:14, SEQ ID NO:15. In some embodiments, the protein or amino acid sequence comprises at least about 70% or 80% sequence identity to SEQ ID NO:1, SEQ ID NO:2, SEQ ID NO:3, SEQ ID NO:4, SEQ ID NO:5, SEQ ID NO:6, SEQ ID NO:7, SEQ ID NO:9, SEQ ID NO:10, SEQ ID NO:11, SEQ ID NO:12, SEQ ID NO:13, SEQ ID NO:14, or SEQ ID NO:15 but is free of SEQ ID NO:1, SEQ ID NO:2, SEQ ID NO:3, SEQ ID NO:4, SEQ ID NO:5, SEQ ID NO:6, SEQ ID NO:7, SEQ ID NO:9, SEQ ID NO:10, SEQ ID NO:11, SEQ ID NO:12, SEQ ID NO:13, SEQ ID NO:14, SEQ ID NO:15, respectively.

In some embodiments, the system is free of SEQ ID NO:1. In some embodiments, the system is free of SEQ ID NO:2. In some embodiments, the system is free of SEQ ID NO:3. In some embodiments, the system is free of SEQ ID NO:4. In some embodiments, the system is free of SEQ ID NO:5. In some embodiments, the system is free of SEQ ID NO:6. In some embodiments, the system is free of SEQ ID NO:7. In some embodiments, the system is free of SEQ ID NO:8. In some embodiments, the system is free of SEQ ID NO:9. In some embodiments, the system is free of SEQ ID NO:10. In some embodiments, the system is free of SEQ ID NO:11. In some embodiments, the system is free of SEQ ID NO:12. In some embodiments, the system is free of SEQ ID NO:13. In some embodiments, the system is free of SEQ ID NO:14. In some embodiments, the system is free of SEQ ID NO:15.

The present invention also provides nucleic acids encoding any of the proteins described above. In some embodiments, the nucleic acid comprises. The present invention also provides vectors comprising any of the nucleic acids described above encoding any of the proteins described above. In some embodiments, the vector is a plasmid. In some embodiments, the vector is a retrovirus.

In some embodiments, the protein comprises at least 80% sequence identity to SEQ ID NO:1, and comprises more than one of the amino acid substitutions in SEQ ID NO:1. In some embodiments, the mutation is a substitution or deletion mutation 540, 558; 561, 563, 727, 782, 784, 785, 787, 789, 794 and/or 795.

The present disclosure relates to a system comprising: (i) a solid support comprising at least one vessel, wherein the vessel comprises a solution comprising a first amino acid sequence that comprises at least about 70% sequence identity to X₁X₂X₃X₄X₅X₆X₇X₈X₉X₁₀X₁₁X₁₂X₁₃X₁₄; or (ii) a solid support comprising a solid surface upon which the first amino acid sequence is absorbed, bound covalently or bound non-covalently, wherein the first amino acid sequence comprises at least about 70% sequence identity to X₁X₂X₃X₄X₅X₆X₇X₈X₉X₁₀X₁₁X₁₂X₁₃X₁₄; wherein X₁=I, V, F, L; X₂=any amino acid; X₃=F; X₄=D; X₅=any amino acid; X₆=M, Q, I; X₇=any amino acid; X₈=R, K; X₉=any amino acid; X₁₀=any amino acid; X₁₁=any amino acid; X₁₂=any amino acid; X₁₃=D; X₁₄=Y, V.

In some embodiments, the solid surface is a magnetic bead, a portion of a column, or a modified or unmodified plastic surface. In some embodiments, the solid surface is a modified plastic surface comprising a linker covalently bound to the first amino acid sequence. In some embodiments, the linker is a second amino acid sequence positioned on the solid surface and bound covalently or non-covalently to the first amino acid sequence. In some embodiments, the linker is an antibody or antibody fragment immobilized to the surface of the solid surface comprising a complementary determinant region (CDR) specific to an the first amino acid sequence.

In some embodiments, the amino acid sequence comprises at least 90% sequence identity to X₁X₂X₃X₄X₅X₆X₇X₈X₉X₁₀X₁₁X₁₂X₁₃X₁₄; wherein X₁=I, V, F, L; X₂=any amino acid; X₃=F; X₄=D; X₅=any amino acid; X₆=M, Q, I; X₇=any amino acid; X₈=R, K; X₉=any amino acid; X₁₀=any amino acid; X₁₁=any amino acid; X₁₂=any amino acid; X₁₃=D; X₁₄=Y, V.

In some embodiments, the first amino acid sequence comprises about 70% sequence identity to an amino acid comprising contiguous amino acids with Formula: X_{B}−[from about 53 to about 56 amino acids]−[[X₁X₂X₃X₄X₅X₆X₇X₈X₉X₁₀X₁₁X₁₂X₁₃X₁₄]; wherein X_{B}=N or P; X₁=I, V, F, or L; X₂=any amino acid; X₃=F; X₄=D; X₅=any amino acid; X₆=M, Q, or I; X₇=any amino acid; X₈=R or K; X₉=any amino acid; X₁₀=any amino acid; X₁₁=any amino acid; X₁₂=any amino acid; X₁₃=D; X₁₄=Y or V.

In some embodiments, the first amino acid sequence comprises at least about 75% sequence identity to an amino acid with contiguous amino acids of Formula III: [X₁ₐX₂ₐX₃ₐX₄ₐX₅ₐX₆ₐ]−[from about 161 to about 164 amino acids]−X_{B}−[from about 53 to about 56 amino acids]−[X₁X₂X₃X₄X₅X₆X₇X₈X₉X₁₀X₁₁X₁₂X₁₃X₁₄]; wherein X₁ₐ=Y; X₂ₐ=any amino acid; X₃ₐ=an amino acid; X₄ₐ=F; X₅ₐ=any amino acid; X₆ₐ=S or G; X_{B}=N or P; X₁=I, V, F, or L; X₂=any amino acid; X₃=F; X₄=D; X₅=any amino acid; X₆=M, Q, or I; X₇=any amino acid; X₈=R or K; X₉=any amino acid; X₁₀=any amino acid; X₁₁=any amino acid; X₁₂=any amino acid; X₁₃=D; X₁₄=Y or V.

In some embodiments, the first amino acid sequence comprises at least about 75% sequence identity to an amino acid with contiguous amino acids of Formula IV: [X_{A}]−[from about 15 to about 20 amino acids]−[X₁ₐX₂ₐX₃ₐX₄ₐX₅ₐX₆ₐ]−[from about 161 to about 164 amino acids]−X_{B}−[from about 53 to about 56 amino acids]−[X₁X₂X₃X₄X₅X₆X₇X₈X₉X₁₀X₁₁X₁₂X₁₃X₁₄]; wherein X_{A}=D or E; X₁ₐ=Y; X₂ₐ=any amino acid; X₃ₐ=an amino acid; X₄ₐ=F; X₅ₐ=any amino acid; X₆ₐ=S or G; X_{B}=N or P; X₁=I, V, F, or L; X₂=any amino acid; X₃=F; X₄=D; X₅=any amino acid; X₆=M, Q, or I; X₇=any amino acid; X₈=R or K; X₉=any amino acid; X₁₀=any amino acid; X₁₁=any amino acid; X₁₂=any amino acid; X₁₃=D; X₁₄=Y or V.

In some embodiments, the first amino acid is chosen from an amino acid sequence comprising at least about 75% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15 or a functional fragment thereof. In some embodiments, the first amino acid is a functional fragment comprising at least about 75% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15. In some embodiments, the first amino acid is chosen from an amino acid sequence comprising a substitution mutation at amino acid number chosen from: 540, 558, 559, 561, 563, 727, 782, 784, 785, 787, 789, 794 and/or 795, in relation to such amino acid numbers identified in any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15.

In some embodiments, the solid support comprises at least one vessel with a volume defined by a contiguous surface forming walls and a base of the vessel. In some embodiments, the solid support is a 6-, 12-, 24-, 48-, or 96-well plate. In some embodiments, the solid support comprises at least one vessel with a volume defined by a first surface that forms a base of the vessel and one or a plurality of secondary surfaces that form walls of the vessel. Some embodiments further comprise a sample.

In some embodiments, the amino acid is associated with a saxitoxin (STX) compound having a structure represented by a formula:

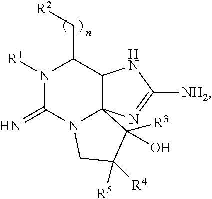

wherein n is selected from 0, 1, and 2; wherein each of R¹ and R³ is independently selected from hydrogen and —OH; wherein R² is selected from hydrogen, —OH, and —OC(O)R¹⁰; wherein R¹⁰, when present, is selected from —NH₂, —CH₃, —NHSO₂H, —NHSO₃⁻, —NHSO₃H, and Ar¹; wherein Ar¹, when present, is a C6 aryl substituted with 0, 1, 2, or 3 groups independently selected from —OH, —SO₃⁻, and —SO₃H; and wherein each of R⁴ and R⁵ is independently selected from hydrogen, —OH, —OSO₃H, and —OSO₃⁻, or a salt thereof.

In some embodiments, the amino acid is associated with a tetrodotoxin (TTX) compound having a structure represented by a formula selected from:

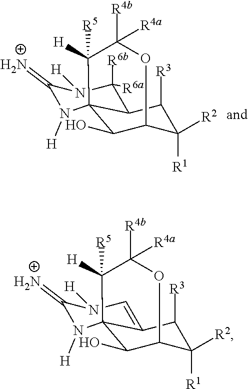

wherein R¹ is selected from hydrogen and —OH; wherein R² is selected from —CH₃, —CH₂OH, and —CH(OH)₂; wherein R³ is hydrogen and wherein each of R^{4a} and R^{4b} together comprise ═O, or wherein R³ and R^{4a} together comprise —O— and wherein R^{4b} is —OH; and wherein R⁵ is —OH and wherein each of R^{6a} and R^{6b} is independently selected from hydrogen and —OH, or wherein R⁵ and R^{6b} together comprise —O— and wherein R^{6a} is hydrogen; or a salt thereof.

The present disclosure also relates to a composition comprising a first amino acid sequence that comprises at least 70% sequence identity to X₁X₂X₃X₄X₅X₆X₇X₈X₉X₁₀X₁₁X₁₂X₁₃X₁₄; wherein X₁=I, V, F, L; X₂=any amino acid; X₃=F; X₄=D; X₅=any amino acid; X₆=M, Q, I; X₇=any amino acid; X₈=R, K; X₉=any amino acid; X₁₀=any amino acid; X₁₁=any amino acid; X₁₂=any amino acid; X₁₃=D; X₁₄=Y, V; and SEQ ID NO:12.

Some embodiments further comprise a targeting domain covalently bound or non-covalently bound to the first amino sequence. In some embodiments, the targeting domain comprises a fluorescent molecule. In some embodiments, targeting domain comprises a dye, quantum dot, streptavidin, biotin, or an enzyme. In some embodiments, the targeting domain comprises a dye intercalated within the amino acid. In some embodiments, the amino acid is immobilized on a solid surface or in solution. In some embodiments, the composition is free of SEQ ID NO: 1, SEQ ID NO:2, SEQ ID NO:11, SEQ ID NO:12, SEQ ID NO:13, SEQ ID NO:14 and SEQ ID NO:15.

In some embodiments, the amino acid is associated with a saxitoxin (STX) compound having a structure represented by a formula:

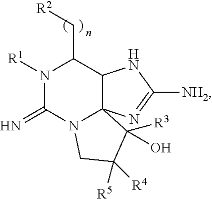

wherein n is selected from 0, 1, and 2; wherein each of R¹ and R³ is independently selected from hydrogen and —OH; wherein R² is selected from hydrogen, —OH, and —OC(O)R¹⁰; wherein R¹⁰, when present, is selected from —NH₂, —CH₃, —NHSO₂H, —NHSO₃⁻, —NHSO₃H, and Ar¹; wherein Ar¹, when present, is a C6 aryl substituted with 0, 1, 2, or 3 groups independently selected from —OH, —SO₃⁻, and —SO₃H; and wherein each of R⁴ and R⁵ is independently selected from hydrogen, —OH, —OSO₃H, and —OSO₃⁻, or a salt thereof.

In some embodiments, the amino acid sequence comprises at least 90% sequence identity to X₁X₂X₃X₄X₅X₆X₇X₈X₉X₁₀X₁₁X₁₂X₁₃X₁₄; wherein X₁=I, V, F, L; X₂=any amino acid; X₃=F; X₄=D; X₅=any amino acid; X₆=M, Q, I; X₇=any amino acid; X₈=R, K; X₉=any amino acid; X₁₀=any amino acid; X₁₁=any amino acid; X₁₂=any amino acid; X₁₃=D; X₁₄=Y, V.

In some embodiments, the first amino acid sequence comprises about 70% sequence identity to an amino acid comprising contiguous amino acids with Formula: X_{B}−[from about 53 to about 56 amino acids]−[[X₁X₂X₃X₄X₅X₆X₇X₈X₉X₁₀X₁₁X₁₂X₁₃X₁₄]; wherein X_{B}=N or P; X₁=I, V, F, or L; X₂=any amino acid; X₃=F; X₄=D; X₅=any amino acid; X₆=M, Q, or I; X₇=any amino acid; X₈=R or K; X₉=any amino acid; X₁₀=any amino acid; X₁₁=any amino acid; X₁₂=any amino acid; X₁₃=D; X₁₄=Y or V.

In some embodiments, the first amino acid sequence comprises at least about 75% sequence identity to an amino acid with contiguous amino acids of Formula III: [X₁ₐX₂ₐX₃ₐX₄ₐX₅ₐX₆ₐ]−[from about 161 to about 164 amino acids]−X_{B}−[from about 53 to about 56 amino acids]−[X₁X₂X₃X₄X₅X₆X₇X₈X₉X₁₀X₁₁X₁₂X₁₃X₁₄]; wherein X₁ₐ=Y; X₂ₐ=any amino acid; X₃ₐ=an amino acid; X₄ₐ=F; X₅ₐ=any amino acid; X₆ₐ=S or G; X_{B}=N or P; X₁=I, V, F, or L; X₂=any amino acid; X₃=F; X₄=D; X₅=any amino acid; X₆=M, Q, or I; X₇=any amino acid; X₈=R or K; X₉=any amino acid; X₁₀=any amino acid; X₁₁=any amino acid; X₁₂=any amino acid; X₁₃=D; X₁₄=Y or V.

In some embodiments, the first amino acid sequence comprises at least about 75% sequence identity to an amino acid with contiguous amino acids of Formula IV: [X_{A}]−[from about 15 to about 20 amino acids]−[X₁ₐX₂ₐX₃ₐX₄ₐX₅ₐX₆ₐ]−[from about 161 to about 164 amino acids]−X_{B}−[from about 53 to about 56 amino acids]-[X₁X₂X₃X₄X₅X₆X₇X₈X₉X₁₀X₁₁X₁₂X₁₃X₁₄]; wherein X_{A}=D or E; X₁ₐ=Y; X₂ₐ=any amino acid; X₃ₐ=an amino acid; X₄ₐ=F; X₅ₐ=any amino acid; X₆ₐ=S or G; X_{B}=N or P; X₁=I, V, F, or L; X₂=any amino acid; X₃=F; X₄=D; X₅=any amino acid; X₆=M, Q, or I; X₇=any amino acid; X₈=R or K; X₉=any amino acid; X₁₀=any amino acid; X₁₁=any amino acid; X₁₂=any amino acid; X₁₃=D; X₁₄=Y or V.

In some embodiments, the first amino acid is chosen from an amino acid sequence comprising at least about 75% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO:13, SEQ ID NO:14 or SEQ ID NO:15 or a functional fragment thereof. In some embodiments, the first amino acid is a functional fragment comprising at least about 75% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO:13, SEQ ID NO:14 or SEQ ID NO:15. In some embodiments, the first amino acid is chosen from an amino acid sequence comprising a substitution mutation at amino acid number chosen from: 540, 558, 559, 561, 563, 727, 782, 784, 785, 787, 789, 794 and/or 795, in relation to such amino acid numbers identified in any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO:13, SEQ ID NO:14 or SEQ ID NO:15.

The present disclosure also relates to a method of detecting the quantity or presence of a paralytic shellfish poisoning (PSP) toxin in a sample, the method comprising: (i) exposing the sample to a composition comprising an amino acid sequence having at least 90% sequence identity to least 90% sequence identity to X₁X₂X₃X₄X₅X₆X₇X₈X₉X₁₀X₁₁X₁₂X₁₃X₁₄; wherein X₁=I, V, F, L; X₂=any amino acid; X₃=F; X₄=D; X₅=any amino acid; X₆=M, Q, I; X₇=any amino acid; X₈=R, K; X₉=any amino acid; X₁₀=any amino acid; X₁₁=any amino acid; X₁₂=any amino acid; X₁₃=D; X₁₄=Y, V.

In some embodiments, the step (i) is performed for a time period sufficient to bind the amino acid to a toxin in the sample. Some embodiments further comprise a step (ii) comprising measuring the amount of amino acid sequence bound to the PSP toxin present in the sample. In some embodiments, the method is performed in an animal, and wherein the step of measuring comprises monitoring the death of an animal exposed to the sample in the presence or absence of the amino acid sequence. In some embodiments, the sample is from a mollusk. In some embodiments, the amino acid sequence comprises at least 90% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO:13, SEQ ID NO:14 or SEQ ID NO:15 or a functional fragment thereof. In some embodiments, the amino acid sequence comprises a sequence chosen from Formula I, Formula II, Formula III or Formula IV.

In some embodiments, the PSP toxin is a saxitoxin (STX) compound having a structure represented by a formula:

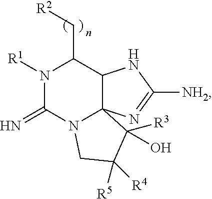

wherein n is selected from 0, 1, and 2; wherein each of R¹ and R³ is independently selected from hydrogen and —OH; wherein R² is selected from hydrogen, —OH, and —OC(O)R¹⁰; wherein R¹⁰, when present, is selected from —NH₂, —CH₃, —NHSO₂H, —NHSO₃⁻, —NHSO₃H, and Ar¹; wherein Ar¹, when present, is a C6 aryl substituted with 0, 1, 2, or 3 groups independently selected from —OH, —SO₃⁻, and —SO₃H; and wherein each of R⁴ and R⁵ is independently selected from hydrogen, —OH, —OSO₃H, and —OSO₃⁻, or a salt thereof.

In some embodiments, the PSP toxin is a tetrodotoxin (TTX) compound having a structure represented by a formula selected from:

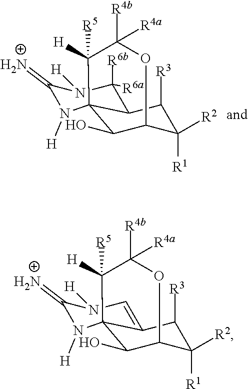

wherein R¹ is selected from hydrogen and —OH; wherein R² is selected from —CH₃, —CH₂OH, and —CH(OH)₂; wherein R³ is hydrogen and wherein each of R^{4a} and R^{4b} together comprise ═O, or wherein R³ and R^{4a} together comprise —O— and wherein R^{4b} is —OH; and wherein R⁵ is —OH and wherein each of R^{6a} and R^{6b} is independently selected from hydrogen and —OH, or wherein R⁵ and R^{6b} together comprise —O— and wherein R^{6a} is hydrogen; or a salt thereof.

In some embodiments, the composition comprises an amino acid sequence covalently bound or non-covalently bound to a targeting domain. In some embodiments, the step (ii) of measuring the amount of amino acid sequence bound to the PSP toxin comprises mixing a known amount of the amino acid sequence and the sample for a time period sufficient to associate the amino acid sequence to the saxitoxin or derivative thereof and measuring the association between the amino acid sequence and the saxitoxin or derivative thereof by one or more of: fluorescence, microscopy, chemiluminescence, elution, wavelength absorbance, or enzymatic cleavage.

The disclosure also relates to a method of diagnosing a subject as contaminated with a toxin, the method comprising: (i) exposing a sample from the subject to a composition comprising an amino acid sequence having at least 70% sequence identity to X₁X₂X₃X₄X₅X₆X₇X₈X₉X₁₀X₁₁X₁₂X₁₃X₁₄; wherein X₁=I, V, F, L; X₂=any amino acid; X₃=F; X₄=D; X₅=any amino acid; X₆=M, Q, I; X₇=any amino acid; X₈=R, K; X₉=any amino acid; X₁₀=any amino acid; X₁₁=any amino acid; X₁₂=any amino acid; X₁₃=D; X₁₄=Y, V; (ii) measuring an association between the amino acid sequence and a paralytic shellfish poisoning (PSP) toxin; and (iii) diagnosing the subject as being contaminated with the toxin if the quantity of association between the amino acid sequence and the PSP toxin is higher than a threshold level of the toxin.

In some embodiments, the step (i) is performed for a time period sufficient to bind the amino acid to the PSP toxin in the sample. Some embodiments further comprise a step, after step (ii) but before step (iii), normalizing quantitative values obtained from measuring the association by subtracting or comparing the quantitative values obtained from the step of measuring to control values of association determined by a control. In some embodiments, the sample is from a mollusk. In some embodiments, the amino acid sequence comprises at least 90% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO:13, SEQ ID NO:14 or SEQ ID NO:15 or a functional fragment thereof.

In some embodiments, the PSP toxin is a saxitoxin (STX) compound having a structure represented by a formula:

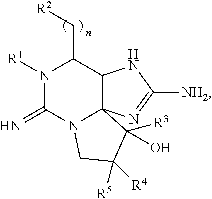

wherein n is selected from 0, 1, and 2; wherein each of R¹ and R³ is independently selected from hydrogen and —OH; wherein R² is selected from hydrogen, —OH, and —OC(O)R¹⁰; wherein R¹⁰, when present, is selected from —NH₂, —CH₃, —NHSO₂H, —NHSO₃⁻, —NHSO₃H, and Ar¹; wherein Ar¹, when present, is a C6 aryl substituted with 0, 1, 2, or 3 groups independently selected from —OH, —SO₃⁻, and —SO₃H; and wherein each of R⁴ and R⁵ is independently selected from hydrogen, —OH, —OSO₃H, and —OSO₃⁻, or a salt thereof.

In some embodiments, the PSP toxin is a tetrodotoxin (TTX) compound having a structure represented by a formula selected from:

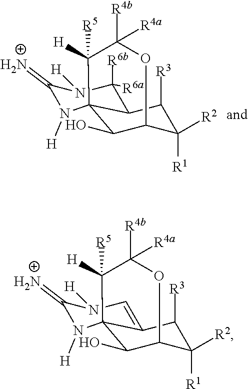

wherein R¹ is selected from hydrogen and —OH; wherein R² is selected from —CH₃, —CH₂OH, and —CH(OH)₂; wherein R³ is hydrogen and wherein each of R^{4a} and R^{4b} together comprise ═O, or wherein R³ and R^{4a} together comprise —O— and wherein R^{4b} is —OH; and wherein R⁵ is —OH and wherein each of R^{6a} and R^{6b} is independently selected from hydrogen and —OH, or wherein R⁵ and R^{6b} together comprise —O— and wherein R^{6a} is hydrogen; or a salt thereof.

In some embodiments, the composition comprises an amino acid sequence covalently bound or non-covalently bound to a targeting domain. In some embodiments, the step of measuring the amount of amino acid sequence bound to the PSP toxin comprises mixing a known amount of the amino acid sequence with the sample for a time period sufficient to associate the amino acid sequence to the PSP toxin and measuring the association between the amino acid sequence and the saxitoxin or derivative thereof by one or a combination of: fluorescence, microscopy, chemiluminescence, elution, wavelength absorbance, or enzymatic cleavage. In some embodiments, the step of measuring further comprises: (a) mixing a known amount of a control amino acid sequence with the sample for a time period sufficient for association of the amino acid sequence with the PSP toxin and measuring the association between the control amino acid sequence and the PSP toxin by one or a combination of: fluorescence, microscopy, chemiluminescence, elution, wavelength absorbance, or enzymatic cleavage; and (b) normalizing the association of the amino acid sequence to the PSP toxin to the association of the control to the PSP toxin.

The present disclosure also relates to a method of assaying the toxicity of a subject comprising: (i) exposing a sample from the subject to a composition comprising an amino acid sequence having at least 70% sequence identity to X₁X₂X₃X₄X₅X₆X₇X₈X₉X₁₀X₁₁X₁₂X₁₃X₁₄; wherein X₁=I, V, F, L; X₂=any amino acid; X₃=F; X₄=D; X₅=any amino acid; X₆=M, Q, I; X₇=any amino acid; X₈=R, K; X₉=any amino acid; X₁₀=any amino acid; X₁₁=any amino acid; X₁₂=any amino acid; X₁₃=D; X₁₄=Y, V; (ii) measuring an association between the amino acid sequence and a paralytic shellfish poisoning (PSP) toxin; and (iii) determining that the subject comprises a toxic substance if the association between the amino acid sequence and the PSP toxin is higher than a threshold level of toxin; or determining that the subject is free of a toxic substance if the association between the amino acid sequence and the PSP toxin is lower than the threshold level of the toxin.

In some embodiments, step (i) is performed for a time period sufficient to bind the amino acid to the PSP toxin in the sample. Some embodiments further comprise a step, after step (ii) but before step (iii), normalizing quantitative values obtained from measuring the association by subtracting or comparing the quantitative values obtained from the step of measuring to control values of association determined by a control. In some embodiments, the sample is from a mollusk. In some embodiments, the amino acid sequence comprises at least 90% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO:13, SEQ ID NO:14 or SEQ ID NO:15 or a functional fragment thereof. In some embodiments, the amino acid sequence comprises a functional fragment of an amino sequence chosen from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO:13, SEQ ID NO:14 or SEQ ID NO: 15.

In some embodiments, the PSP toxin is a saxitoxin (STX) compound having a structure represented by a formula:

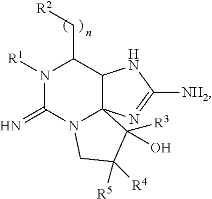

wherein n is selected from 0, 1, and 2; wherein each of R¹ and R³ is independently selected from hydrogen and —OH; wherein R² is selected from hydrogen, —OH, and —OC(O)R¹⁰; wherein R¹⁰, when present, is selected from —NH₂, —CH₃, —NHSO₂H, —NHSO₃⁻, —NHSO₃H, and Ar¹; wherein Ar¹, when present, is a C6 aryl substituted with 0, 1, 2, or 3 groups independently selected from —OH, —SO₃⁻, and —SO₃H; and wherein each of R⁴ and R⁵ is independently selected from hydrogen, —OH, —OSO₃H, and —OSO₃⁻, or a salt thereof.

In some embodiments, the PSP toxin is a tetrodotoxin (TTX) compound having a structure represented by a formula selected from:

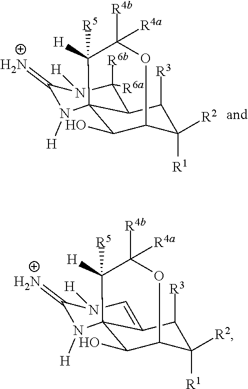

wherein R¹ is selected from hydrogen and —OH; wherein R² is selected from —CH₃, —CH₂OH, and —CH(OH)₂; wherein R³ is hydrogen and wherein each of R^{4a} and R^{4b} together comprise ═O, or wherein R³ and R^{4a} together comprise —O— and wherein R^{4b} is —OH; and wherein R⁵ is —OH and wherein each of R^{6a} and R^{6b} is independently selected from hydrogen and —OH, or wherein R⁵ and R^{6b} together comprise —O— and wherein R^{6a} is hydrogen; or a salt thereof.

In some embodiments, the composition comprises an amino acid sequence covalently bound or non-covalently bound to a targeting domain. In some embodiments, the step of measuring the amount of amino acid sequence bound to the PSP toxin comprises mixing a known amount of the amino acid sequence with the sample for a time period sufficient to associate the amino acid sequence to the PSP toxin and measuring the association between the amino acid sequence and the PSP toxin by one or a combination of: fluorescence, microscopy, chemiluminescence, elution, wavelength absorbance, or enzymatic cleavage.

In some embodiments, the step of measuring further comprises: (a) mixing a known amount of a control amino acid sequence with the sample for a time period sufficient for association of the amino acid sequence with the PSP toxin and measuring the association between the control amino acid sequence and the PSP toxin by one or a combination of: fluorescence, microscopy, chemiluminescence, elution, wavelength absorbance, or enzymatic cleavage; and (b) normalizing the association of the amino acid sequence to the PSP toxin the association of the control to the PSP toxin.

The disclosure also relates to a method of identifying a population of contaminated animals comprising toxic levels of a paralytic shellfish poisoning (PSP) toxin comprising: (i) exposing a subject to a sample from the population in the presence of a composition comprising an amino acid sequence having at least 70% sequence identity to Formula I, II, III or IV; (ii) detecting an association between the amino acid sequence and the PSP toxin; and (iii) identifying the population as being contaminated if the association between the amino acid sequence and the PSP toxin is higher than a threshold level of toxin; or identifying the population as not being contaminated if the association between the amino acid sequence and the PSP toxin is lower than the threshold level of the toxin. Some embodiments further comprise a step of (iv) correlating a contamination status of the population with a contamination status of a known location or geography based upon the source of the sample.

The disclosure also relates to a kit comprising: (a) one or a plurality of the compositions; or a system; and a first container comprising instructions; or (b) a first container comprising: a solid support comprising one or a plurality of reaction vessels; and a composition comprising one or a plurality of vials or tubes, each vial or tube comprising one or a combination the disclosed compositions; or (c) a first container comprising the disclosed systems; or a first container comprising one or a plurality of vials or tubes, each vial or tube comprising one or a combination the disclosed compositions; and a second container comprising instructions.

The disclosure also relates to a diagnostic test comprising (i) filter; and (ii) a saxiphilin or functional fragment thereof comprising a detection molecule covalently or non-covalently attached to said saxiphilin or functional fragment thereof. In some embodiments, the saxiphilin or functional fragment thereof is embedded in the filter.

The disclosure also relates to a method of preparing a sample comprising or suspected of comprising a toxin comprising (i) exposing homogenate to a filter; and (ii) analyzing the homogenate for presence of a toxin. In some embodiments, prior to step (i) the sample is homogenized to form a homogenate. In some embodiments, the filter comprises a saxiphilin or functional fragment thereof. In some embodiments, the saxiphilin comprises a detection molecule covalently or non-covalently attached to said saxophilin or functional fragment thereof. In some embodiments, the step of analyzing comprises detecting whether a toxin bound to said saxiphilin or functional fragment thereof.

### BRIEF DESCRIPTIONS OF THE DRAWINGS

FIG. 1. Alanine scan of RcSxph binding.

FIGS. 1A, and 1B. Exemplar thermofluor (TF) assay results for RcSxph (FIG. 1A) in the presence of the indicated concentrations of STX (left) and TTX (right) and Select RcSxph mutants (FIG. 1B) in the presence of STX. STX and TTX concentrations are 0 nM (black), 19.5 nM (blue), 625 nM (cyan), 5000 nM (orange), and 20000 nM (red). Dashed lines indicate □Tm. FIG. 1C. F-STX diagram. STX and fluorescein (F) moieties are highlighted blue and yellow, respectively. FIG. 1D. Exemplar Fluorescence polarization (FP) binding curves and Kds for RcSxph and the indicated mutants. FIG. 1E. Comparison of RcSxph mutant □Tm and □□G values (line y=3.49-0.7523x, R²=0.886). FIG. 1F. Exemplar isotherms for titration of 100 μM STX into 10 μM RcSxph. 100 μM STX into 10 μM RcSxph F561A, 100 μM STX into 10 μM RcSxph E540D, and 300 μM STX into 30 μM RcSxph D794E. Kd and □H values are indicated. FIG. 1G. Comparison of □G_{ITC} for STX and □G_{FP} for F-STX for RcSxph and mutants. Purple box highlights region of good correlation. Orange box indicates region outside of the ITC dynamic range. (line shows x=y)

FIGS. 2A through 2D. Energetic fingerprint of STX recognition by RcSxph.

FIG. 2A. □□G comparisons for the indicated RcSxph STX binding pocket mutants relative to wild-type RcSxph. Colors indicate: □□□□G<−1 kcal mol⁻¹ (blue), −1≤□□G≤0 kcal mol⁻¹ (light blue), 0≥□□G≥1 kcal mol⁻¹ (yellow), 1≥□□G≥2 kcal mol⁻¹ (orange), 2≥□□G≥3 kcal mol⁻¹ (red orange), and □□G≥3 (red). FIG. 2B. Energetic map of alanine scan mutations on STX binding to the RcSxph STX binding pocket (PDB:6O0F) (Yen et al.). Second shell sites are in italics. Colors are as in ‘A’. C and D, Structural interactions of STX with C, RcSxph (PDB:6O0F) (Yen et al.) and D, human Na_{V}1.7 (PDB:6J8G) (Shen et al. 2019). Residues that are energetically important for the STX interaction are shown in space filling. Na_{V}1.7 selectivity filter ‘DEKA’ ring residues are shown (white). Italics indicate corresponding residue numbers for rat Na_{V}1.4 (Thomas-Tran and Du Bois).

FIG. 3. Structures of enhanced affinity RcSxph mutants.

FIG. 3A. Superposition of the STX binding pockets of RcSxph-Y558A (purple) and the RcSxph-Y558A:STX complex (light blue). FIG. 3B. Superposition of the STX binding pockets ofRcSxph-Y558I (pale yellow) and the RcSxph-Y558I:STX complex (splitpea). FIG. 3C. Superposition of the STX binding pockets of STX bound complexes of RcSxph (PDB: 6O0F) (Yen et al.), RcSxph-Y558A (purple), and RcSxph-Y558I (splitpea). STX from the RcSxph is firebrick.

FIG. 4. RcSxph mutants have differential effects on PtNa_{V}1.4 rescue from STX block.

FIG. 4A-E, Exemplar two-electrode voltage clamp recordings of PtNa_{V}14 expressed in Xenopus oocytes in the presence of 100 nM STX and indicated [Sxph]:[STX] ratios for RcSxph (FIG. 4A), RcSxph E540A (FIG. 4B), RcSxph Y558I (FIG. 4C), RcSxph P727A (FIG. 4D), and RcSxph I782A (FIG. 4E). Inset shows the stimulation protocol. FIG. 4F. [Sxph]:[STX] dose response curves for RcSxph (black, open circles), RcSxph E540A (inverted triangles), RcSxph Y558I (triangles), RcSxph P727A (diamonds), and RcSxph I782A (squares) in the presence of 100 nM STX. Lines show fit to the Hill equation.

FIG. 5. Sxph family member properties.

FIG. 5A. Comparison of the human transferrin (TF) Fe³⁺ ligand positions (UniProtKB: P02787), RcSxph STX binding motif residues (Yen et al.), and number of Thy1 domains for Sxphs from R. catesbaiana (PDB:600D) (Yen et al.), N. parkeri (NCBI: XP_018410833.1) (Yen et al.), M. aurantiaca, D. tinctorius, O. sylvatica, R. imitator, P. terribilis, E. tricolor, A. femoralis, R. marinus, B. bufo (NCBI:XM_040427746.1), and B. garagarizans. (NCBI:XP_044148290.1). TF Fe³⁺ (orange) and carbonate (blue) ligands are indicated. Blue highlights indicate residue conservation. FIG. 5B. Comparison of STX binding pocket for the indicated Sxphs. Numbers denote RcSxph positions. Conserved residues are highlighted. Asterix indicates second shell sites. FIG. 5C. Exemplar TF curves for NpSxph, RiSxph, OsSxph, and MaSxph in the presence of the indicated concentrations of STX (purple box) or TTX. (green box) □Tm values are indicated. FIG. 5D. Exemplar FP binding curves and Kds for NpSxph (green), RiSxph (blue), OsSxph (orange), and MaSxph (purple). FIG. 5E. Exemplar NpSxph I559Y TF curves in the presence of the indicated concentrations of STX (purple box) or TTX (green box) and FP binding (green). □□Tm and Kd values are indicated. Error bars are S.E.M.

FIG. 6. NpSxph and NpSxph:STX structures.

FIG. 6A. Cartoon diagram of the NpSxph:STX complex. N1 (light green), N2 (green), Thy1-1 (light orange), Thy1-2 (orange), C1 (marine), and C2 (light blue) domains are indicated. STX (pink) is shown in space filling representation. FIG. 6B. Comparison of STX binding pocket for apo-NpSxph (yellow) and NpSxph:STX (marine). STX (pink) is shown as ball and stick. FIG. 6C. Comparison of NpSxph (marine) and RcSxph (orange) (PDB:6O0F) (Yen et al.) STX binding sites. STX from NpSxph and RcSxph complexes is pink and orange, respectively. FIG. 6D. Comparison of NpSxph (marine) and RcSxph-Y558I (splitpea) STX binding sites. STX from NpSxph and RcSxph-Y558I complexes is pink and splitpea, respectively. RcSxph and RcSxph-Y558I residue numbers in FIG. 6C and FIG. 6D are indicated in italics.

FIG. S1. RcSxph thermofluor (TF) assay.

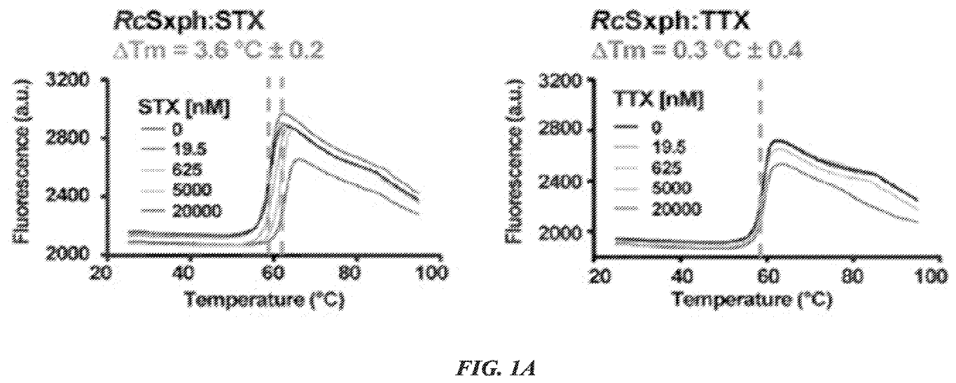

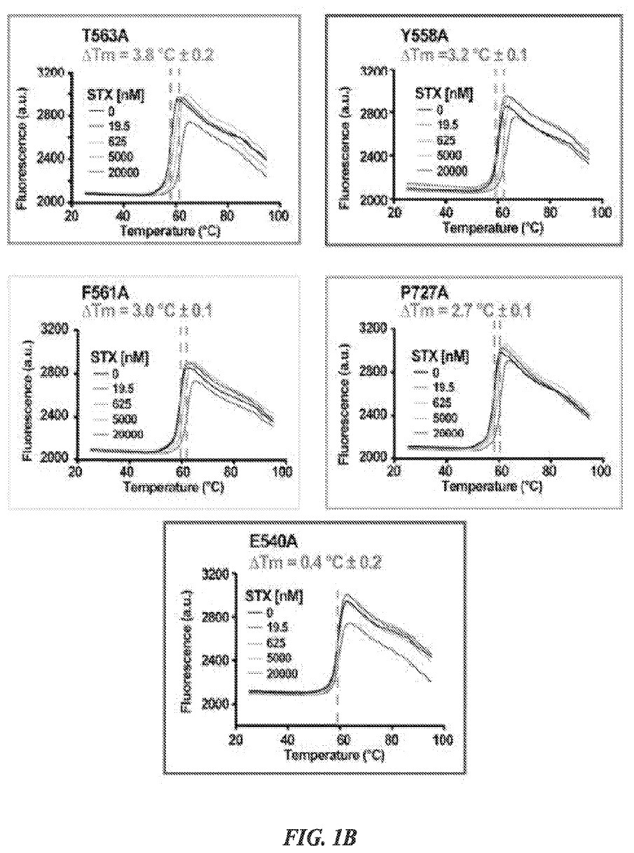

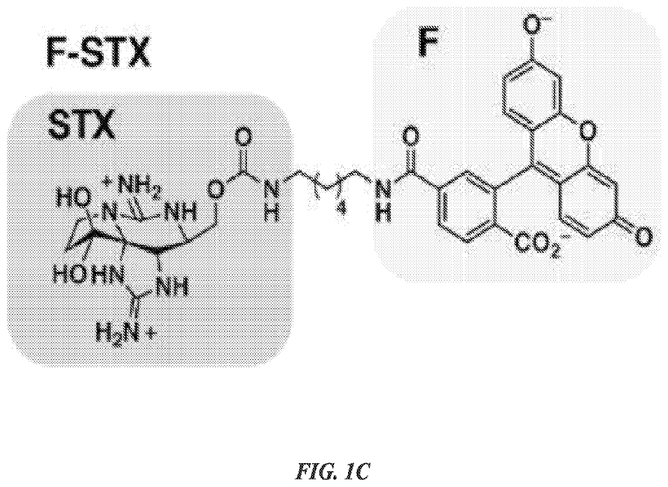

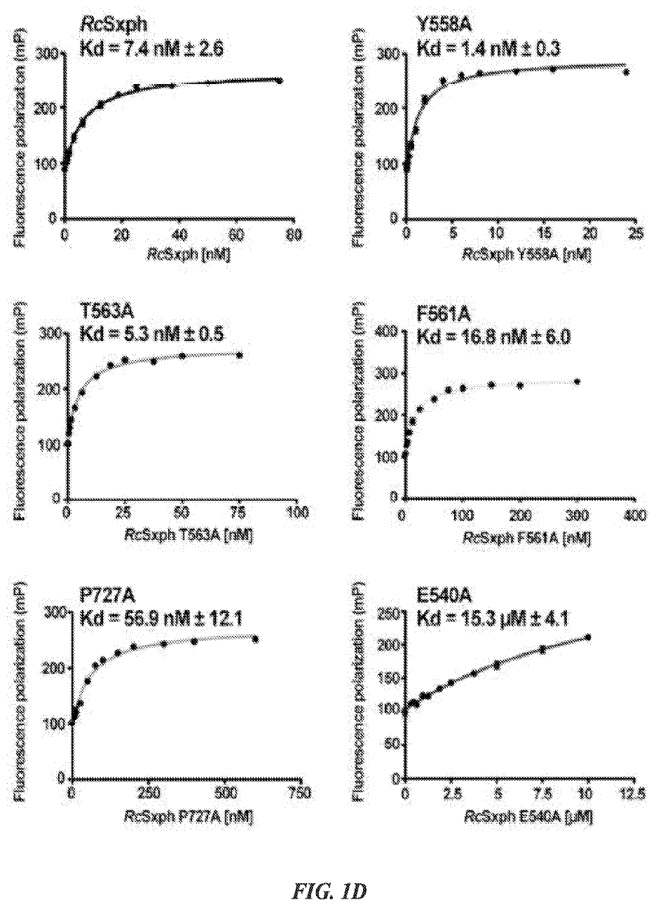

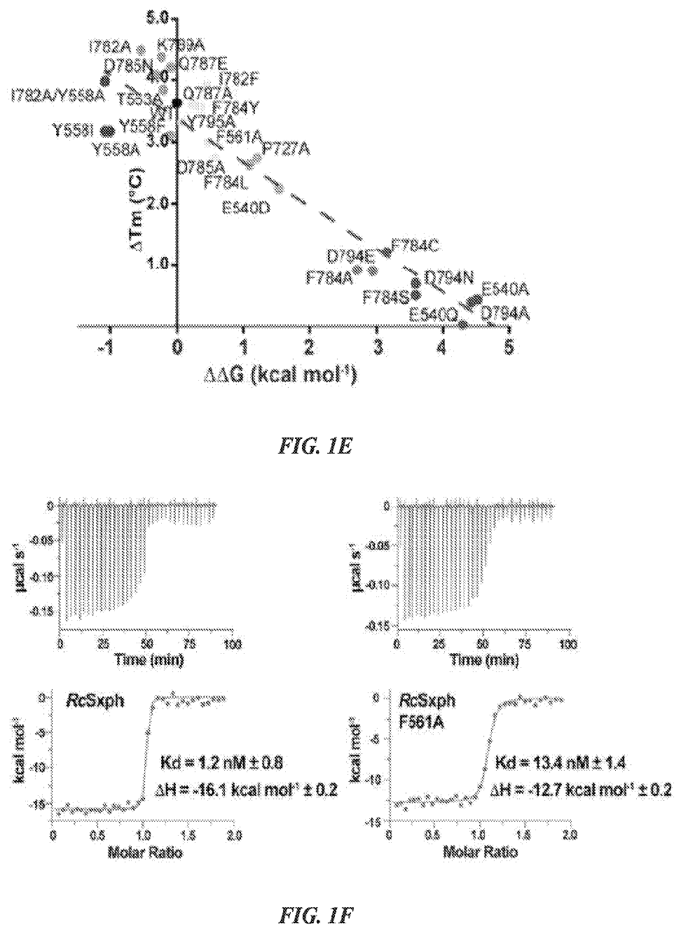

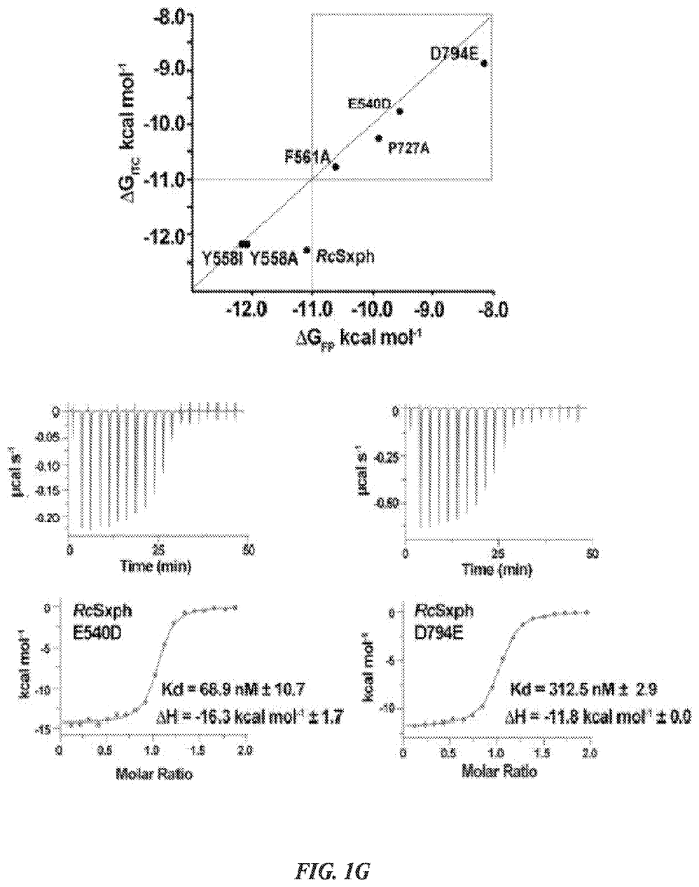

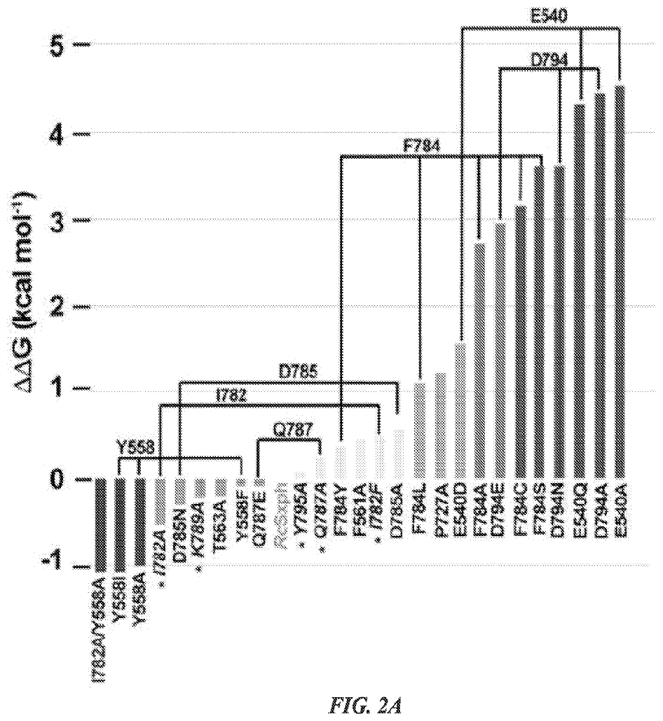

FIG. S1A. Exemplar thermofluor (TF) assay results for RcSxph in the presence of the indicated concentrations of STX. Curves for RcSxph, E540A, P727A, Y558A, F561A, and T563A are identical to those shown in FIGS. 1A and 1B. DTm values are indicated. FIG. SIB. Baseline Tm values for RcSxph and the indicated mutants. FIG. S1C. Plot of Tm vs. DTm for the proteins in FIG. S1B. Error bars are S.E.M.

FIG. S2. F-STX NMR spectrum.

FIG. S2. ¹H NMR (600 MHz, D₂O) δ 8.05 (d, J=8.1 Hz, 1H), 7.94 (d, J=8.9 Hz, 1H), 7.48 (s, 1H), 6.95 (d, J=9.0 Hz, 2H), 6.79 (s, 2H), 6.67 (dt, J=9.1, 2.2 Hz, 2H), 4.60 (d, J=1.2 Hz, 1H), 4.09-4.05 (m, 1H), 3.89 (dd, J=11.6, 5.2 Hz, 1H), 3.70 (dt, J=10.1, 5.5 Hz, 1H), 3.64 (dd, J=8.7, 5.4 Hz, 1H), 3.47-3.42 (m, 1H), 3.27 (t, J=6.6 Hz, 2H), 2.97-2.89 (m, 2H), 2.36-2.33 (m 1H), 2.30-2.24 (m, 1H), 1.48-1.45 (m, 2H), 1.32-1.29 (m, 2H), 1.25-1.21 (m, 4H) ppm

FIG. S3. Structure of the RcSxph:F-STX: complex.

FIG. S3A. Exemplar electron density (Is) for RcSxph (dark gray) and F-STX (light gray). FIG. S3B. RcSxph:F-STX: B-factors for the F-STX ligand and select binding site residues. FIG. S3C. Superposition of the STX binding sites of the RcSxph:F-STX; and RcSxph:STX (PDB:6O0F) (Yen et al.) complexes.

FIG. s4. RcSxph fluorescence polarization (FP) assay.

FIG. S4. Exemplar FP binding curves and Kds for RcSxph and the indicated mutants. Curves for RcSxph, E540A, P727A, Y558A, F561A, and T563A are identical to those shown in FIG. 1D. Colored boxes and lines in correspond to DDG classifications in Table 1. Error bars are S.E.M.

FIG. S5. RcSxph and NpSxph Isothermal titration calorimetry.

FIG. S5. Exemplar ITC isotherms for 100 μM STX into 10 μM RcSxph Y558A (FIG. S5A), 100 μM STX into 10 μM RcSxph Y558I (FIG. S5B), 100 μM STX into 10 μM RcSxph P727A (FIG. S5C), 100 μM STX into 9.7 μM NpSxph (FIG. S5D), and 100 μM STX into 7.9 μM NpSxph I559Y (FIG. S5E). FIG. S5F. Comparison of DG_{ITC} for STX and DG_{FP} for F-STX for RcSxph, NpSxph, and indicated mutants. Purple box highlights region of good correlation. Orange box indicates region outside of the ITC dynamic range. RcSxph data are identical to FIG. 1G.

FIG. S6. RcSxph Y558A and RcSxph-Y558I structures and STX complexes.

FIG. S6. Exemplar electron density (1.5 s) for RcSxph Y558A (purple)(S6A), RcSxph-Y558A:STX (light blue)(S6B), RcSxph-Y558I (pale yellow)(S6C), and RcSxph-Y558I:STX (splitpea)(S6D). Select residues and STX are indicated.

FIG. S7. Frog Sxph sequence alignment.

FIG. S7. Sxph sequence alignment for RcSxph, NpSxph, MaSxph, DtSxph, OsSxph, RiSxph, PtSxph, EtSxph, and AfSxph. Domains and secondary structure are from RcSxph. N1 (dark green), N2 (light green), Thy1 domains (orange), C1 (marine), C2 (cyan). STX binding site residues are indicated by stars and colored based on the alanine scan results in Table 1. Residues corresponding to transferrin Fe³⁺ and carbonate ligands are indicated by orange and blue hexagons, respectively and highlighted (Yen et al., Lambert et al.).

FIG. S8. Toad Sxph sequence alignment.

Sxph sequence alignment for RcSxph, and toad saxiphilins RmSxph, BbSxph (NCBI:XM_040427746.1), and BgSxph(NCBI:XP_044148290.1). Domains and secondary structure are from RcSxph. N1 (dark green), N2 (light green), Thy1 domains (orange), C1 (marine), C2 (cyan). STX binding site residues are indicated by stars and colored based on the alanine scan results in Table 1. Residues corresponding to transferrin Fe³⁺ and carbonate ligands (Yen et al., Lambert et al.) are indicated by orange and blue hexagons, respectively and highlighted. Only beginning and ends of the Thy1 domains are shown. Total number of Thy1 domains are indicated.

FIG. s9. Thy1 domain sequence alignment.

Thy1 domains from RcSxph, NpSxph, MaSxph, DtSxph, OsSxph, RiSxph, PtSxph, EtSxph, AfSxph, RmSxph, BbSxph, and BgSxph and the type (1A or 1B) are shown. Secondary structure from RcSxph Thy1-1 is shown. Cysteine are highlighted. SS4-SS8 indicate disulfide numbers from RcSxph. Loop regions are indicated.

FIG. S10. NpSxph structure and comparisons with RcSxph.

FIG. S10A and B. Exemplar electron density for NpSxph (2Fo-Fc, 1.5 s, grey) and (Fo-Fc, 3.0 s, green) (FIG. S10A), NpSxph:STX (2Fo-Fc, 1.5 s, grey). NpSxph (marine), STX (pink), and PEG400 (yellow) (FIG. S10B) are shown. STX (pink) from the NpSxph:STX complex is shown in FIG. S10A to compare with the PEG400 position. Select residues are labelled. FIG. S10C. NpSxph and RcSxph superposition using the C-lobes. N- and C-lobes are green/light green and marine/light blue for NpSxph and RcSxph, respectively. Arrow indicate relationships between NpSxph and RcSxph N-lobes. FIG. S10D. Superposition of NpSxph (green) and RcSxph (light green) N-lobes. FIG. S10E. Superposition of NpSxph (marine) and RcSxph (light blue) C-lobes. FIG. S10F. Cartoon diagram of NpSxph and RcSxph superposition from FIG. S10C showing the change in Thy1 domain positions. NpSxph Thy1 domains (orange) and RcSxph Thy1 domains (magenta) are indicated. FIG. S10G. Cartoon diagram of NpSxph and RcSxph Thy1 domains superposed on Thy1-1. NpSxph and RcSxph Thy1-1 and Thy1-2 are light orange and pink and orange and magenta, respectively. FIG. S10H. Superposition of individual NpSxph and RcSxph Thy1-1 and Thy1-2 domains. Disulfide bonds are indicated.

FIG. S11. Structure of the NpSxph:F-STX complex.

FIG. S11A. Exemplar electron density for NpSxph:F-STX(2Fo-Fc, 1.5 s, grey). NpSxph (cyan) and F-STX (orange). FIG. S11B. Comparison of NpSxph:STX (marine) and NpSxph:F-STX STX binding sites. STX from NpSxph is pink. F-STX is orange. Select residues are indicated.

### DETAILED DESCRIPTION OF THE EMBODIMENTS

Before the present compositions and methods are described, it is to be understood that this disclosure is not limited to the particular molecules, compositions, methodologies or protocols described, as these may vary. It is also to be understood that the terminology used in the description is for the purpose of describing the particular versions or embodiments only, and is not intended to limit the scope of the present disclosure which will be limited only by the appended claims. It is understood that these embodiments are not limited to the particular methodology, protocols, cell lines, vectors, and reagents described, as these may vary. It also is to be understood that the terminology used herein is for the purpose of describing particular embodiments only, and is not intended to limit the scope of the present embodiments or claims.

Unless defined otherwise, all technical and scientific terms used herein have the same meanings as commonly understood by one of ordinary skill in the art. Although any methods and materials similar or equivalent to those described herein can be used in the practice or testing of embodiments of the present disclosure, the preferred methods, devices, and materials are now described. All publications mentioned herein are incorporated by reference. Nothing herein is to be construed as an admission that the disclosure is not entitled to antedate such disclosure by virtue of prior disclosure.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of.” “Consisting essentially of,” when used in the claims, shall have its ordinary meaning as used in the field of patent law.

The term “about” as used herein when referring to a measurable value such as an amount, a temporal duration, and the like, is meant to encompass variations of 20%, +10%, ±5%, ±1%, ±0.9%, ±0.8%, ±0.7%, ±0.6%, ±0.5%, ±0.4%, ±0.3%, ±0.2% or 0.1% from the specified value, as such variations are appropriate to perform the disclosed methods.

As used herein, the phrase “integer from X to Y” means any integer that includes the endpoints. That is, where a range is disclosed, each integer in the range including the endpoints is disclosed. For example, the phrase “integer from X to Y” discloses 1, 2, 3, 4, or 5 as well as the range 1 to 5.

As used herein, “substantially equal” means within a range known to be correlated to an abnormal or normal range at a given measured unit. In some embodiments, “substantially equal” means that the associated term is from about +/−10% of where an equal value would be associated with whatever metric is modified by the term. For example, if a control sample is from a diseased patient, substantially equal is, in some embodiments, within an abnormal range+/−10% of the abnormal value. If a control sample is from a patient known not to have the condition being tested, substantially equal is within a normal range for that given metric. In some embodiments, the amount of expression of toxin (e.g. STX or TTX) detected by any of the methods disclosed herein is from about 1.01 to about 2.00 times the amount of expression of the toxin disclosed herein in order for the diagnosis of a toxin exposure or contamination to be made. In some embodiments, the amount of toxin detected by any of the methods disclosed herein is from about 1.01 to about 1.50 times the amount of toxin disclosed herein in order for the diagnosis of toxin exposure to be made.

As used herein, the term “subject,” “individual” or “patient,” used interchangeably, means any animal, including mammals, such as mice, rats, other rodents, rabbits, dogs, cats, swine, cattle, sheep, horses, or primates, such as humans. The term “subject” is used, in some embodiments, throughout the specification to describe an animal from which a sample is taken. In some embodiment, the subject is a human. For diagnosis of those conditions which are specific for a specific subject, such as a human being, the term “patient” may be interchangeably used. In some instances in the description of the present invention, the term “patient” will refer to human patients suffering from a particular disease or disorder. In some embodiments, the subject may be a human suspected of having or being identified as at risk to a terminal condition or disorder. In some embodiments, the subject may be a mammal which functions as a source of the isolated sample of biopsy or bodily fluid. In some embodiments, the subject may be a non-human animal from which a sample of biopsy or bodily fluid is isolated or provided. In some embodiments, the subject is a mollusk.

As used herein, the terms “biologically significant” refers to an amount or concentration of enzymatic reaction product or enzyme in a sample whose quantity of binding that is detected and is statistically significant as compared to a control when the amount or concentration is normalized for a control. In some embodiments, the terms is used to describe the amount of toxin that is present in a sample at a level sufficient to cause a dysfunctional biological effect. In some embodiments, the biologically significant amount of amino acid sequence (or saxiphilin) is the amount sufficient to bind or neutralize toxins disclosed herein. In some embodiments, the biologically significant amount of amino acid sequence disclosed herein is the amount sufficient to characterize a sample as toxic. In some embodiments, the biologically significant amount of toxin is the amount sufficient to cause PSP. In some embodiments, the biologically significant amount of toxin is the amount sufficient to characterize a sample as being toxic to a subject.

As used herein, the term “kit” refers to a set of components provided in the context of a system for delivering materials or diagnosing a subject with having been contaminated with a disclosed toxin or exposed to a disclosed toxin. Such delivery systems may include, for example, systems that allow for storage, transport, or delivery of various diagnostic reagents (e.g., oligonucleotides, enzymes, extracellular matrix components etc. in appropriate containers) and/or supporting materials (e.g., buffers, media, cells, written instructions for performing the assay etc.) from one location to another. For example, in some embodiments, kits include one or more enclosures (e.g., boxes) containing relevant reaction reagents and/or supporting materials. As used herein, the term “fragmented kit” refers to a diagnostic assay comprising two or more separate containers that each contain a subportion of total kit components. Containers may be delivered to an intended recipient together or separately. For example, a first container may contain a petri dish or polystyrene plate for use in a cell culture assay, while a second container may contain cells, such as control cells. As another example, the kit may comprise a first container comprising a solid support such as a chip or slide with one or a plurality of ligands with affinities to one or a plurality of biomarkers disclosed herein and a second container comprising any one or plurality of reagents necessary for the detection and/or quantification of the amount of biomarkers in a sample. The term “fragmented kit” is intended to encompass kits containing Analyte Specific Reagents (ASR's) regulated under section 520(e) of the Federal Food, Drug, and Cosmetic Act, but are not limited thereto. Indeed, any delivery system comprising two or more separate containers that each contain a sub-portion of total kit components are included in the term “fragmented kit.” In contrast, a “combined kit” refers to a delivery system containing all components in a single container (e.g., in a single box housing each of the desired components). The term “kit” includes both fragmented and combined kits.

As used herein, “cell culture” means growth, maintenance, transfection, or propagation of cells, tissues, or their products. As used herein, “culture medium” refers to any solution capable of sustaining the growth of the targeted cells either in vitro or in vivo, or any solution with which targeted cells or exogenous nucleic acids are mixed before being applied to cells in vitro or to a patient in vivo. In some embodiments, culture medium means solution capable of sustaining the growth of the targeted cells either in vitro.

As used herein, the term “animal” includes, but is not limited to, humans and non-human vertebrates such as wild animals, rodents, such as rats, ferrets, and domesticated animals, and farm animals, such as dogs, cats, horses, pigs, cows, sheep, and goats. In some embodiments, the animal is a mammal. In some embodiments, the animal is a human. In some embodiments, the animal is a non-human mammal.

As used herein, the term “mammal” means any animal in the class Mammalia such as rodent (i.e., a mouse, a rat, or a guinea pig), a monkey, a cat, a dog, a cow, a horse, a pig, or a human. In some embodiments, the mammal is a human. In some embodiments, the mammal refers to any non-human mammal. The present disclosure relates to any of the methods or compositions of matter disclosed herein wherein the sample is taken from a mammal or non-human mammal. The present disclosure relates to any of the methods or compositions of matter disclosed herein wherein the sample is taken from a human or non-human primate.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the terms “treat,” “treated,” or “treating” can refer to therapeutic treatment and/or prophylactic or preventative measures wherein the object is to prevent or slow down (lessen) an undesired physiological condition, disorder or disease, or obtain beneficial or desired clinical results. For purposes of the embodiments described herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment can also include eliciting a clinically significant response without excessive levels of side effects. Treatment also includes prolonging survival as compared to expected survival if not receiving treatment.

As used herein, the terms “diagnose,” “diagnosing,” or variants thereof refer to identifying the nature of a physiological condition, disorder or disease.

Any probes may be used in concert with any of the devices, systems, kits, or methods disclosed herein. As used herein, the term “probe” refers to any molecule that may bind or associate, indirectly or directly, covalently or non-covalently, to any of the saxiphilins and/or reaction products disclosed herein and whose association or binding is detectable using the methods disclosed herein. In some embodiments, the probe is a fluorogenic, fluorescent, or chemiluminescent probe, an antibody, or an absorbance-based probe. In some embodiments, an absorbance-based probe, for example the chromophore pNA (para-nitroanaline), may be used as a probe for detection and/or quantification of a toxin disclosed herein. In some embodiments, the probe comprises an amino acid sequence that is a natural or non-natural ligand of an toxin disclosed herein and/or an analog or salt thereof, including those analogs that comprise at least about 70%, at least about 75%, at least about 80%, at least about 85%, at least about 87%, at least about 90%, at least about 95%, at least about 96%, at least about 97%, at least about 98%, or at least about 99% sequence identity to amino acids SEQ ID NO: 1, 2, 3, 4, 5, 6, 7, 8, 9, 10, 11, 12, 13, 14 or 15 as indicated in the Table 3 below. A probe may be immobilized, adsorbed, or otherwise non-covalently bound to a solid surface, such that upon exposure to an enzyme for a time period sufficient to associate with the toxin. In some embodiments, association of the toxin to the amino acid sequence causes a biological change in the nature or chemical availability of one or more probes such that the biological change enables detection of the association event. For instance, if the step of detecting comprises use of FRET, cleavage of the amino acid compositions disclosed herein cause one of the chromophore to emit a fluorescent light under exposure to a wavelength sufficient to activate such a fluorescent molecule. The intensity, length, or amplitude of a wavelength emitted from fluorescent marker can be measured and is, in some embodiments, proportional to the presence, absence or quantity of toxin present in the reaction vessel, thereby the quantity of enzyme can be determined from detection of the intensity of or fluorescence at a known wavelength of light.

An “activity-based probe,” as used herein, refers to a certain embodiment of probe comprising a small molecule that binds to or has affinity for a molecule such as an amino acid that binds a toxin in the presence of such an amino acid sequence, such that its bound or unbound state confers an activity readout observable by a user. In some embodiments, the activity-based probe covalently or non-covalently binds to a toxin disclosed herein or derivative herein. In some embodiments, the binding of the activity-based probe modifies the physical or biological activity of the toxin. In some embodiments, the activity-based probe can be fluorescent or chemiluminescent. In some embodiments, the activity-based probe has a measurable activity of one value if the enzyme is inactive and another measurable activity if in an activated state.

As used herein, the terms “fluorogenic” and “fluorescent” probe refer to any molecule (dye, quantum dot, peptide, or fluorescent marker) that emits a known and/or detectable wavelength of light upon exposure to a known wavelength of light. In some embodiments, the saxiphilins or peptides with known cleavage sites recognizable by any of the enzymes expressed by one or a plurality of mucinous cysts are covalently or non-covalently attached to a fluorogenic probe. In some embodiments, the attachment of the fluorogenic probe to the saxiphilin creates a chimeric molecule capable of a fluorescent emission or emissions upon exposure of the amino acid sequence to the toxin and the known wavelength of light, such that exposure to the toxin creates a reaction product which is quantifiable in the presence of a fluorimeter. In some embodiments, light from the fluorogenic probe is fully quenched upon exposure to the known wavelength of light before association of the disclosed toxins and the disclosed amino acid sequences the fluorogenic probe emits a known wavelength of light, the intensity of which is quantifiable by absorbance readings or intensity levels in the presence of a fluorimeter and after enzymatic cleavage of the substrate. In some embodiments, the fluorogenic probe is a coumarin-based dye or rhodamine-based dye with fluorescent emission spectra measureable or quantifiable in the presence of or exposure to a predetermined wavelength of light. In some embodiments, the fluorogenic probe comprises rhodamine. In some embodiments, the fluorogenic probe comprises rhodamine-100. Coumarin-based fluorogenic probes are known in the art, for example in U.S. Pat. Nos. 7,625,758 and 7,863,048, which are herein incorporated by reference in their entireties. In some embodiments, the fluorogenic probes are a component to, covalently bound to, non-covalently bound to, intercalated with one or a plurality of amino acid sequences or toxins disclosed herein. In some embodiments, the fluorogenic probes are chosen from ACC or AMC. In some embodiments, the fluorogenic probe is a fluorescein molecule. In some embodiments, the fluorogenic probe is capable of emitting a resonance wave detectable and/or quantifiable by a fluorimeter after exposure to one or a plurality of toxins disclosed herein. “Fluorescence microscopy,” which uses the fluorescence to generate an image, may be used to detect the presence, absence, or quantity of a fluorescent probe. In some embodiments, fluorescence microscopy comprises measuring fluorescence resonance energy transfer (FRET) within a FRET-based assay.

A “chemiluminescent probe” refers to any molecule (dye, peptide, or chemiluminescent marker) that emits a known and/or detectable wavelength of light as the result of a chemical reaction. Chemiluminescence differs from fluorescence or phosphorescence in that the electronic excited state is the product of a chemical reaction rather than of the absorption of a photon. Non-limiting examples of chemiluminescent probes are luciferin and aequorin molecules. In some embodiments, a chemiluminescent molecule is covalently or non-covalently attached to a saxiphilin disclosed herein or toxin, such that the excited electronic state can be quantified to determine directly to the amino acid sequences disclosed, such as a saxiphilin, is in a reaction vessel, or, indirectly, by quantifying the amount of association that was produced between the amino acid sequences and the toxins after activation of the probe on the amino acid sequence or a portion of the sequence.

As used herein, an “enzyme” can be any partially or wholly proteinaceous molecule which carries out a chemical reaction in a catalytic manner upon exposure to a substrate. Such enzymes can be native enzymes, fusion enzymes, proenzymes, apoenzymes, denatured enzymes, famesylated enzymes, ubiquitinated enzymes, fatty acylated enzymes, gerangeranylated enzymes, GPI-linked enzymes, lipid-linked enzymes, prenylated enzymes, naturally-occurring or artificially-generated mutant enzymes, enzymes with side chain or backbone modifications, enzymes having leader sequences, and enzymes complexed with non-proteinaceous material, such as proteoglycans, proteoliposomes. Enzymes can be made by any means, including natural expression, promoted expression, cloning, various solution-based and solid-based peptide syntheses, and similar methods known to those of skill in the art. In some embodiments, signal peptides or probes bound to the amino acids or toxins disclosed herein.

As used herein, “conservative” amino acid substitutions may be defined as set out in Tables A, B, or C below. Saxiphilin sequences disclosed herein may include those amino acid sequences that include or a plurality of conservative substitutions have been introduced by modification of polynucleotides encoding polypeptides of the invention. Amino acids can be classified according to physical properties and contribution to secondary and tertiary protein structure. A conservative substitution is recognized in the art as a substitution of one amino acid for another amino acid that has similar properties. Exemplary conservative substitutions are set out in Table A.

TABLE A
Conservative Substitutions I

| Side Chain Characteristics | Amino Acid Aliphatic |
|---|---|
| Non-polar | G A P I L V F |
| Polar - uncharged | CSTMNQ |
| Polar - charged | D E K R |
| Aromatic | H F W Y |
| Other | NQDE |

Alternately, conservative amino acids can be grouped as described in Lehninger, (Biochemistry, Second Edition; Worth Publishers, Inc. NY, N.Y. (1975), pp. 71-77) as set forth in Table B.

TABLE B
Conservative Substitutions II

| Side Chain Characteristic | Amino Acid |
|---|---|
| Non-polar (hydrophobic) | |
| Aliphatic: | A L I V P. |
| Aromatic: | F W Y |
| Sulfur-containing: | M |
| Borderline: | G Y |
| Uncharged-polar | |
| Hydroxyl: | S T Y |
| Amides: | N Q |
| Sulfhydryl: | C |
| Borderline: | G Y |
| Positively Charged (Basic): | K R H |
| Charged (Acidic): | D E |

Alternately, exemplary conservative substitutions are set out in Table C.

TABLE C
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |

As used herein, the term “sample” refers generally to a limited quantity of something which is intended to be similar to and represent a larger amount of that thing. In the present disclosure, a sample is a collection, fluid, blood, swab, brushing, scraping, biopsy, removed tissue, surgical resection that is to be tested. In some embodiments, the sample is a bivalve comprising a toxin or an extract from a homogenized or nonhomogenized animal, such as a mollusk or bivalve. As used herein, “control sample” or “reference sample” refer to samples with a known presence, absence, or quantity of substance being measured, that is used for comparison against an experimental sample. In some embodiments, the sample may be a tissue sample from a mollusk or a human. In some embodiments, the human is exhibiting signs of toxin poisoning or suspected of ingesting a contaminated mollusk or portion of a contaminated mollusk.

In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc. in some embodiments, the methods disclosed herein do not comprise a processed sample.

In some embodiments, the sample is homogenized. It may be homogenized by any method known in the art. It may be homogenized with a mixer, a tissue grinder or a high pressure homogenizer. Samples can be homogenized for any length of time necessary to create a uniform homogenate.

A filter as used herein may be any filter known in the art, including a cation exchange membrane filter, such as Nafion or similar perfluorinated ionomers. Anion exchange membranes may also be used, as well as various polymeric hydrogels such as acrylamide, poly(ethylene glycol) diacrylate, poly(2-hydroxylethyl methacrylate), or poly(vinyl alcohol). Additionally, other exemplary embodiments may include mechanisms for quantitative analysis of the color change by means of photodiodes and sensors or microfluidic devices that require smaller amounts of reagent and samples.

A “score” is a numerical value that may be assigned or generated after normalization of the value based upon the presence, absence, or quantity of substrates or enzymes disclosed herein. In some embodiments, the score is normalized in respect to a control raw data value.

In some embodiments, a “toxin” is a naturally occurring non-peptidyl neurotoxin in the family of saxitoxin. In some embodiments, a “toxin” is a neurotoxin. In some embodiments, a “toxin” is saxitoxin. In some embodiments, a “toxin” is a saxitoxin family member. There are 57 known saxitoxin family members (Wiese et al., 2010 March Drugs 8:2185-2211, which is hereby incorporated by reference in its entirety). A toxin may comprise one or more than one saxitoxin family member.

In some embodiments, a toxin is a compound having a structure represented by a formula:

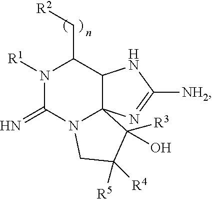

wherein n is selected from 0, 1, and 2; wherein each of R¹ and R³ is independently selected from hydrogen and —OH; wherein R² is selected from hydrogen, —OH, and —OC(O)R¹⁰; wherein R¹⁰, when present, is selected from —NH₂, —CH₃, —NHSO₂H, —NHSO₃⁻, —NHSO₃H, and Ar¹; wherein Ar¹, when present, is a C6 aryl substituted with 0, 1, 2, or 3 groups independently selected from —OH, —SO₃⁻, and —SO₃H; and wherein each of R⁴ and R⁵ is independently selected from hydrogen, —OH, —OSO₃H, and —OSO₃⁻, or a salt thereof.

In some embodiments, a toxin is a structure represented by a formula selected from:

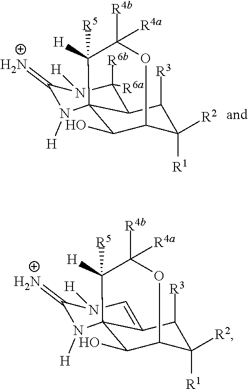

wherein R¹ is selected from hydrogen and —OH; wherein R² is selected from —CH₃, —CH₂OH, and —CH(OH)₂; wherein R³ is hydrogen and wherein each of R^{4a} and R^{4b} together comprise ═O, or wherein R³ and R^{4a} together comprise —O— and wherein R^{4b} is —OH; and wherein R⁵ is —OH and wherein each of R^{6a} and R^{6b} is independently selected from hydrogen and —OH, or wherein R⁵ and R^{6b} together comprise —O— and wherein R^{6a} is hydrogen; or a salt thereof.

In some embodiments, the disclosure relates to a method of treating a subject exposed to a toxin. In some embodiments, the disclosure relates to a method of detecting a toxin in a sample from a human. In some embodiments, the disclosure relates to a method of detecting a toxin in a sample from a mollusk.

TABLE 5
Saxitoxin structures

| | | |
|---|---|---|
| Saxitoxin | STX | 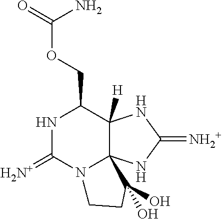 |
| Decarbamoyl Saxitoxin | dcSTX | 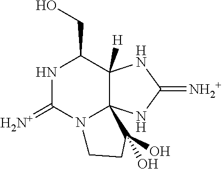 |
| Neosaxitoxin | neoSTX | 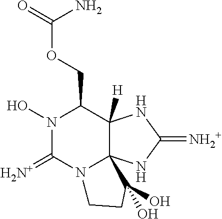 |
| Decarbamoyl Neosaxitoxin | dc-neoSTX | 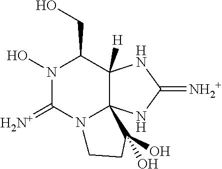 |
| Gonyautoxin IV | GTX5 (B1) | 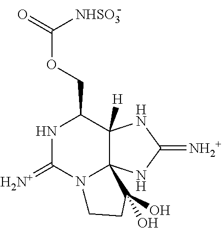 |
| | GTX6 | 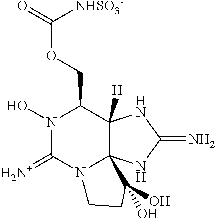 |
| | GTX2/3 | 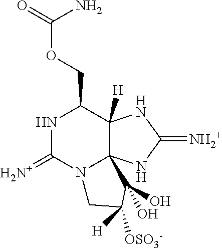 |
| | | 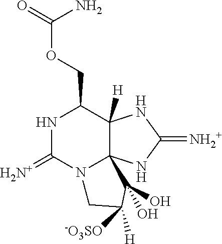 |
| | dcGTX2/3 | 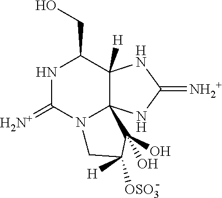 |
| | | 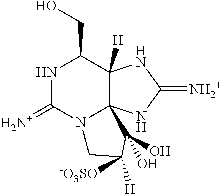 |
| | C1/C2 | 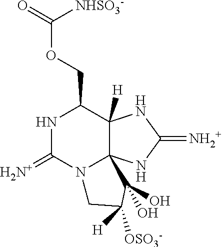 |
| | | 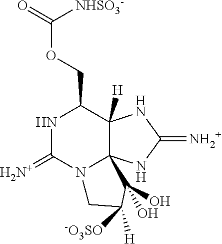 |
| | GTX1/4 | 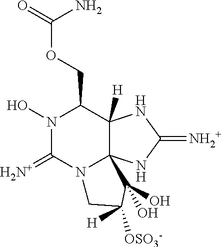 |
| | | 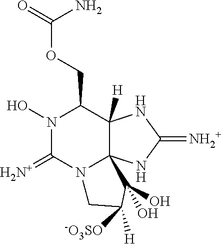 |
| | TTX | 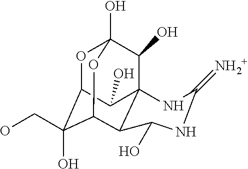 [SMILES: [NH2+]=C1NC(O)C2C3OC4(O)OC([C@@H](O)C2(N1)[C@@H]4O)C3(O)CO] |
| | C13-OBz | 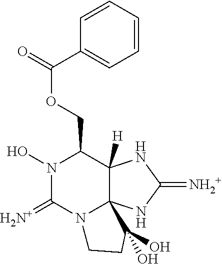 |
| | C13-OAc | 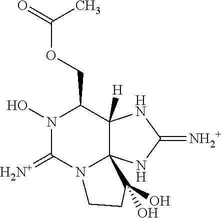 |
| | C13-O-2- CH3-Ph | 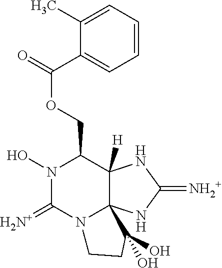 |
| | C13-0-2- F-Ph | 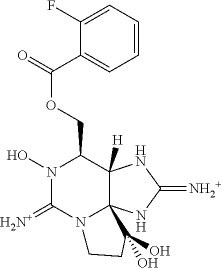 |
| | C13-O-3- CH3-Ph | 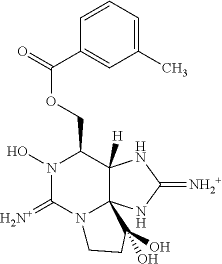 |
| | C13-O-3- F-Ph | 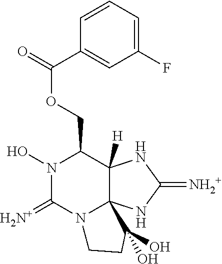 |
| | C13-O-4- CH3-Ph | 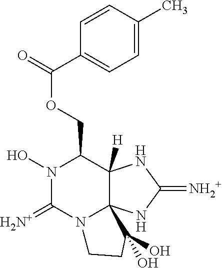 |
| | C13-0-4- F-Ph | 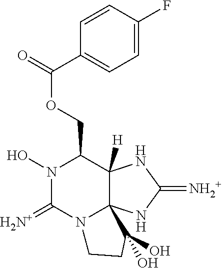 |
| | C13-0-4- OH-Ph | 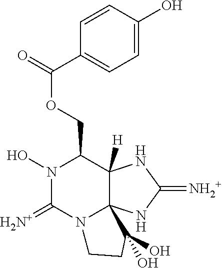 |
| | C13-O-3,4- (OH)2-Ph | 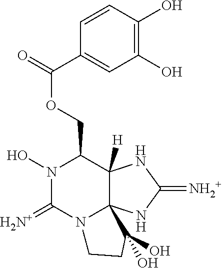 |

The disclosure relates to a system, composition, and series of methods ofusing the systems and compositions for the analysis of a sample from a subject to accurately diagnose, prognose, or classify the subject with exposed to a toxin disclosed herein. The disclosure also relates to a system, composition, and series of methods of using the systems and compositions for the analysis of a sample from a subject to accurately diagnose, prognose, or classify the subject as contaminated with a toxin disclosed herein or that causes PSP. The disclosure relates to a system, composition, and series of methods of using the systems and compositions for the analysis of a sample from a subject to accurately diagnose, prognose, or classify the subject with PSP or exhibiting the signs of PSP. In some embodiments, the system of the present invention comprises a means of detecting and/or quantifying or observing data comprising morphological features, the expression of protein, and/or the expression of nucleic acids in a plurality of cells; and correlating that data with a subject's medical history to predict clinical outcome, treatment plans, preventive medicine plans, or effective therapies.

The “percent identity” or “percent homology” of two polynucleotide or two polypeptide sequences is determined by comparing the sequences using the GAP computer program (a part of the GCG Wisconsin Package, version 10.3 (Accelrys, San Diego, Calif)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (http://www.ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001.

As used herein, “specific for” or “specifically binds to” means that the binding affinity of a substrate to a specified target nucleic acid sequence, such as a tripeptidyl peptidase, is statistically higher than the binding affinity of the same substrate to a generally comparable, but non-target amino acid sequence. The substrate's Kd to each nucleotide sequence can be compared to assess the binding specificity of the substrate to a particular target nucleotide sequence.

Human or non-human variants of the enzymes above are contemplated by the methods, systems, and devices disclosed herein. Variants of these enzymes include sequences that are at least 70% homologous or identical to the human sequences above. As used herein, the term “variants” is intended to mean substantially similar sequences. For nucleic acid molecules, a variant comprises a nucleic acid molecule having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” nucleic acid molecule or polypeptide comprises a naturally occurring nucleotide sequence or amino acid sequence, respectively. For nucleic acid molecules, conservative variants include those sequences that, because of the degeneracy of the genetic code, encode the amino acid sequence of one of the polypeptides of the disclosure. Variant nucleic acid molecules also include synthetically derived nucleic acid molecules, such as those generated, for example, by using site-directed mutagenesis but which still encode a protein of the disclosure. Generally, variants of a particular nucleic acid molecule or amino acid sequence of the disclosure will have at least about 70%, 75%, 80%, 85%, 87%, 90%, 91%, 92%, 93%, 94%, 95%, 96%, 97%, 98%, 99% or more sequence identity to that particular polynucleotide as determined by sequence alignment programs and parameters as described elsewhere herein.

Variants of a particular nucleic acid molecule of the disclosure (i.e., the reference amino acid sequence) can also be evaluated by comparison of the percent sequence identity between the polypeptide encoded by a variant nucleic acid molecule and the polypeptide encoded by the reference nucleic acid molecule. Percent sequence identity between any two polypeptides can be calculated using sequence alignment programs and parameters described elsewhere herein. Where any given pair of nucleic acid molecule of the disclosure is evaluated by comparison of the percent sequence identity shared by the two polypeptides that they encode, the percent sequence identity between the two encoded polypeptides is at least about 70%, 75%, 80%, 85%, 87%, 90%, 91%, 92%, 93%, 94%, 95%, 96%, 97%, 98%, 99% or more sequence identity. In some embodiments, the term “variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present disclosure are biologically active, that is they continue to possess the desired biological activity of the native protein as described herein. Such variants may result from, for example, genetic polymorphism or from human manipulation. Biologically active variants of a protein of the disclosure will have at least about 70%, 75%, 80%, 85%, 87%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the disclosure may differ from that protein by as few as 1-15 amino acid residues, as few as 1-10, such as 6-10, as few as 20, 15, 10, 9, 8, 7, 6, 5, as few as 4, 3, 2, or even 1 amino acid residue. The proteins or polypeptides of the disclosure may be altered in various ways including amino acid substitutions, deletions, truncations, and insertions. Methods for such manipulations are generally known in the art. For example, amino acid sequence variants and fragments of the proteins can be prepared by mutations in the nucleic acid sequence that encode the amino acid sequence recombinantly.

The disclosure relates to a system comprising a solid substrate such as a plastic plate or dish comprising at least one vessel with a sidewall and bottom surface defining a volume into which a sample maybe tested. The surface comprises a substrate either covalently or noncovalently bound to the bottom surface or in suspension within the vessel, such that after exposure to a toxin, a reaction takes place between the saxiphilin and a toxin or variant thereof in a sample, a reaction product may be detected using standard detection techniques. In some embodiments, the reaction product may be detected by an antibody.

Any probe disclosed herein may be an antibody. The term “antibody” as used herein refers to a polypeptide or group of polypeptides that are comprised of at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. As used herein, a “targeted binding agent” is an antibody, or binding fragment thereof, that preferentially binds to a target site. In one embodiment, the targeted binding agent is specific for only one target site. In other embodiments, the targeted binding agent is specific for more than one target site. In one embodiment, the targeted binding agent may be a monoclonal antibody and the target site may be an epitope. “Epitope” refers to that portion of an antigen or other macromolecule capable of forming a binding interaction that interacts with the variable region binding pocket of an antibody. “Binding fragments” of an antibody are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. An antibody substantially inhibits adhesion of a receptor to a counter-receptor when an excess of antibody reduces the quantity of receptor bound to counter-receptor by at least about 20%, 40%, 60% or 80%, and more usually greater than about 85% (as measured in an in vitro competitive binding assay). An antibody may be oligoclonal, a polyclonal antibody, a monoclonal antibody, a chimeric antibody, a CDR-grafted antibody, a multi-specific antibody, a bi-specific antibody, a catalytic antibody, a chimeric antibody, a humanized antibody, a fully human antibody, an anti-idiotypic antibody and antibodies that can be labeled in soluble or bound form as well as fragments, variants or derivatives thereof, either alone or in combination with other amino acid sequences provided by known techniques. An antibody may be from any species. The term antibody also includes binding fragments of the antibodies of the invention; exemplary fragments include Fv, Fab, Fab′, single stranded antibody (svFC), dimeric variable region (Diabody) and di-sulphide stabilized variable region (dsFv). As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99% sequence identity to the antibodies or immunoglobulin molecules described herein. In particular, conservative amino acid replacements are contemplated. Conservative replacements are those that take place within a family of amino acids that have related side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are an aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding function or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. See, for example, Bowie et al. Science 253:164 (1991), which is incorporated by reference in its entirety. In some embodiments, the antibody fragments, analogs thereof of the disclosure are antibodies, antibody fragments, analogs thereof bind a toxin disclosed herein. In some embodiments, the antibody fragments, analogs thereof of the disclosure are antibodies, antibody fragments, analogs thereof that bind to a toxin or portion thereof. In some embodiments, the antibody fragments, analogs thereof of the disclosure are antibodies, antibody fragments, analogs thereof that bind to a toxin. In some embodiments, the system comprises a solid substrate spotted in a patterned or non-patterned formation at discrete locations on the solid substrate by one or a plurality of substrates for a toxin. In other embodiments, the system comprises a solid substrate spotted in a patterned or non-patterned formation at discrete locations on the solid substrate by one or a plurality of substrates for a toxin; and the system further comprises one or a plurality of antibodies, antibody fragments, or analogs of the antibodies that bind to a reaction fragment resulting from the cleavage of a substrate exposed to a toxin (or a variant or functional fragment thereof) or that bind directly to a toxin (or a variant or functional fragment thereof).

In some instances, it may be desired to modify the detection probes so that they are more readily able to bind to an analyte or a reaction product. In such instances, the detection probes may be modified with certain specific binding members that are adhered thereto to form conjugated probes. For instance, the detection probe may be conjugated with antibodies as are further described below that are specific to a toxin. The detection probe antibody may be a monoclonal or polyclonal antibody or a mixture(s) or fragment(s) thereof.

The antibodies may generally be attached to the detection probes using any of a variety of well-known techniques. For instance, covalent attachment of the antibodies to the detection probes (e.g., particles) may be accomplished using carboxylic, amino, aldehyde, bromoacetyl, iodoacetyl, thiol, epoxy and other reactive or linking functional groups, as well as residual free radicals and radical cations, through which a protein coupling reaction may be accomplished. A surface functional group may also be incorporated as a functionalized co-monomer as the surface of the detection probe may contain a relatively high surface concentration of polar groups. In addition, although detection probes are often functionalized after synthesis, such as with poly(thiophenol), the detection probes may be capable of direct covalent linking with an antibody without the need for further modification. For example, in one embodiment, the first step of conjugation is activation of carboxylic groups on the probe surface using carbodiimide. In the second step, the activated carboxylic acid groups are reacted with an amino group of an antibody to form an amide bond. The activation and/or antibody coupling may occur in a buffer, such as phosphate-buffered saline (PBS) (e.g., pH of 7.2) or 2-(N-morpholino) ethane sulfonic acid (MES) (e.g., pH of 5.3). The resulting detection probes may then be contacted with ethanolamine, for instance, to block any remaining activated sites. Overall, this process forms a conjugated detection probe, where the antibody is covalently attached to the probe. Besides covalent bonding, other attachment techniques, such as physical adsorption, may also be utilized in the present invention.

In one embodiment, the antibody may be detectably labeled by linking to an enzyme. The enzyme, in turn, when later exposed to a substrate or reaction product or enzyme disclosed herein, will react with a substrate or reaction product or enzyme disclosed herein in such a manner as to produce a chemical moiety which may be detected as, for example, by spectrophotometric or fluorometric means. Examples of enzymes which may be used to detectably label the antibodies as herein described include malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase.

In some embodiments, in vivo or in vitro methods are performed to detect the presence, absence or quantity of one or a plurality of toxins. In some embodiments, any of the disclosed methods or series of methods comprise exposing a sample or tissue in situ with one or a plurality of antibodies, optionally tagged with a visual detection agent such as a probe or fluorophore, which has binding affinity for one or a plurality of the toxins disclosed herein. Antibodies suitable for practicing the methods of the invention may be monoclonal and multivalent, and may be human, humanized or chimeric antibodies, comprising single chain antibodies, Fab fragments, F(ab′) fragments, fragments produced by a Fab expression library, and/or binding fragments of any of the above. In certain embodiments of the invention, the antibodies are human antigen-binding antibody fragments of the present invention and include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, CH3 and CL domains. Also included in the disclosure are antigen-binding fragments comprising any combination of variable region(s) with a hinge region, CH1, CH2, CH3 and CL domains. Preferably, the antibodies are human, murine (e.g., mouse and rat), donkey, sheep, rabbit, goal, guinea pig, camelid, horse, or chicken. As used herein, “human” antibodies include antibodies having the amino acid sequence of a human immunoglobulin and include antibodies isolated from human immunoglobulin libraries, from human B cells, or from animals transgenic for one or more human immunoglobulins.

### Systems

Embodiments of the present invention include a multi-wavelength fluorescence-based diagnostic and/or spectroscopy strategy that combines biochemical reactions elements and software components to provide a system suited for diagnosis. A further preferred embodiment utilizes Raman spectral measurements to provide diagnostic information. Multimodal illumination and detection techniques employing a combination of fluorescence, reflectance, and Raman measurements can also be used. In any of the methods disclosed herein, a procedure and one or more spectral detectors to detect fluorescence spectral data or images, Raman spectral data or images, and reflectance spectral data or images are obtained. Preferably, a Raman light source has an excitation wavelength of at least 700 nm. A spectrometer with one or more wavelength dispersing elements and one or more detector elements can be used to generate spectral data or spectral images. Additional Raman measurements including resonance Raman, surface enhanced Raman, and graphene enhanced Raman spectral measurements can be used for certain applications.

Provided herein are systems for detecting the presence, absence or quantity of a toxin in a sample through toxin-saxiphilin specificity. In some embodiments, peptide-based technologies are used for analysis of toxin-saxiphilin specificity. In some embodiments, the system comprising one or a plurality of discrete locations on a solid support upon which (i) antibodies for saxiphilins are immobilized; (ii) saxiphilins specific to a toxin or functional fragments thereof are immobilized; and/or (iii) a combination of the above (i) and (ii) are immobilized. In some embodiments, the toxin is STX or TTX or a deriviative thereof. In some embodiments, the toxins detected in a sample are saxitoxins or derivatives thereof and saxiphilins specific to those toxins (or functional fragments or variants thereof) are immobilized to a solid support. In some embodiments, the discrete location is in or proximate to a well. In some embodiments, the system comprises a solid support comprising a series of wells or vessels to perform the enzymatic reaction in solution. In some embodiments, the system comprises saxiphilins specific to the above-identified toxins that are fused to or noncovalently bound to one or a plurality of probes, such that after the enzymatic reaction takes place, a probe can be used to detect the presence, absence or quantity of reaction products made as a result of the presence, absence or quantity of toxin in the sample. In some embodiments, the reaction product are cleaved fragments of saxiphilins, the detection of which can be used to correlate the presence, absence, detection or quantification of toxin in the sample.

In some embodiments, the enzymatic assay system of the disclosure is a fluorescence-based enzymatic assay for detecting the presence, absence or quantity of a toxin. In some embodiments, the system of the disclosure comprises internally quenched FRET substrates that incorporated the P4 to P4′ amino acids from the specificity profile of toxin. The FRET substrates contained a 7-amino-4-methylcoumarin (AMC) fluorophore and a dinitrophenol (DNP) quencher positioned at opposing termini, such that protease cleavage yields a fluorescence signal that can be easily detected with a simple benchtop microplate reader. In some embodiments, the system of the disclosure comprises an internally quenched substrate specific for a toxin which contains the selected P3-P4′ sequences and the AMC fluorophore conjugated to the side-chain amine of a lysine residue in P1 to maintain the free N-terminal amine for recognition by a toxin. In some embodiments, the fluorophore is covalently bound to saxiphilin. In some embodiments, the fluorophore is non-covalently bound to the saxiphilin. In some embodiments, the quencher is covalently bound to saxiphilin. In some embodiments, the quencher is non-covalently bound to saxiphilin.

In some embodiments, the system of the disclosure comprises one or a plurality of probes and/or stains that bind to at least one toxin and/or functional fragment thereof. In some embodiments, the probe and stain is one or a plurality of fluorescently labeled or chemiluminescent saxiphilins specific for at least one toxin and/or functional fragment thereof. In some embodiments, the system of the disclosure comprises one or a plurality of substrates specific to at least one toxin and/or functional fragment thereof. In some embodiments, the one or plurality of saxiphilins are fluorogenic, fluorescent, or chemiluminescent fluorescently labeled. In some embodiments, the one or plurality of saxiphilins are covalently bound to a fluorescent molecule. In some embodiments, the one or plurality of saxiphilins are non-covalently bound to a fluorescent molecule. In some embodiments, the one or plurality of saxiphilins are internally quenched FRET saxiphilins comprising a 7-amino-4-methylcoumarin (AMC) fluorophore and a dinitrophenol (DNP) quencher positioned at opposing termini. In some embodiments, the one or plurality of saxiphilins are internally quenched saxiphilins comprising the selected P3-P4′ sequences and the AMC fluorophore conjugated to the side-chain amine of a lysine residue in P1 to maintain the free N-terminal amine for recognition by a toxin. In some embodiments, the fluorophore is covalently bound to the saxiphilins. In some embodiments, the fluorophore is non-covalently bound to the saxiphilins. In some embodiments, the quencher is covalently bound to the saxiphilins. In some embodiments, the quencher is non-covalently bound to the saxiphilins.

In some embodiments, the one or plurality of saxiphilins are chosen from a substrate having the amino acid sequence of [insert claim language here]

In some embodiments, the one or plurality of saxiphilins are chosen from a amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or a variant that comprises at least about 70%, 80%, 87%, 90%, 95%, 96%, 97%, 98%, or 99% sequence identity to the amino acid sequences above.

TABLE 3
Saxiphilin Sequences

| SEQ | | | | |
|---|---|---|---|---|
| ID | | | Common | |
| NO. | Saxiphilin | Organism | names | Amino acid sequence |
| 1 | RcSxph | Rana | American | MAPTFQTALFFTIISLSFAAPNAKQVRWCAISDLE |
| | | (Lithobates) | bullfrog, | QKKCNDLVGSCNVPDITLVCVLRSSTEDCMTAIKD |
| | | catesbeianus | Bullfrog | GQADAMFLDSGEVYEASKDPYNLKPIIAEPYSSNR |
| | | | | DLQKCLKERQQALAKKMIGHYIPQCDEKGNYQPQQ |
| | | | | CHGSTGHCWCVNAMGEKISGTNTPPGQTRATCERH |
| | | | | ELPKCLKERQVALGGDEKVLGRFVPQCDEKGNYEP |
| | | | | QQFHGSTGYSWCVNAIGEEIAGTKTPPGKIPATCQ |
| | | | | KHDLVTTCHYAVAMVKKSSAFQFNQLKGKRSCHSG |
| | | | | VSKTDGWKALVTVLVEKKLLSWDGPAKESIQRAMS |
| | | | | KFFSVSCIPGATQTNLCKQCKGEEGKNCKNSHDEP |
| | | | | YYGNYGAFRCLKEDMGDVAFLRSTALSDEHSEVYE |
| | | | | LLCPDNTRKPLNKYKECNLGTVPAGTVVTRKISDK |
| | | | | TEDINNFLMEAQKRQCKLFSSAHGKDLMFDDSTLQ |
| | | | | LALLSSEVDAFLYLGVKLFHAMKALTGDAHLPSKN |
| | | | | KVRWCTINKLEKMKCDDWSAVSGGAIACTEASCPK |
| | | | | GCVKQILKGEADAVKLEVQYMYEALMCGLLPAVEE |
| | | | | YHNKDDFGPCKTPGSPYTDFGTLRAVALVKKSNKD |
| | | | | INWNNIKGKKSCHTGVGDIAGWVIPVSLIRRQNDN |
| | | | | SDIDSFFGESCAPGSDTKSNLCKLCIGDPKNSAAN |
| | | | | TKCSLSDKEAYYGNQGAFRCLVEKGDVAFVPHTVV |
| | | | | FENTDGKNPAVWAKNLKSEDFELLCLDGSRAPVSN |
| | | | | YKSCKLSGIPPPAIVTREESISDVVRIVANQQSLY |
| | | | | GRKGFEKDMFQLFSSNKGNNLLENDNTQCLITFDR |
| | | | | QPKDIMEDYFGKPYYTTVYGASRSAMSSELISACT |
| | | | | IKHC* |
| 2 | NpSxph | Nanorana | High | MALTFHTALYFTIVGLSFAASDARHVQWCTISHLE |
| | | parkeri | Himalaya | QKKCNDLVGSCNVPDITLACVYRSSTENCMAAIKD |
| | | | frog, | GQADAMFLDSGDVYKASLDHYNLKPIIAEPYSLHR |
| | | | Xizang | ELTKCLKHRQESLGGDKMVKGRYIPQCDEKGNYHP |
| | | | Plateau | VQCHASTGYCWCVNANGEKIEGTNTTPVQTPPTCP |
| | | | frog, | SQVLTKCLKERQEALGGKRIAIGRYIPQCDEQGNY |
| | | | Parker's | RPMQCHGSTGYCWCVNAIGEKIEGTNTPPGNTQPT |
| | | | slow | CQSHDWDTCHYAVAVVKNSSTFQFGQLKGKRSCHS |
| | | | frog, | GLSKTDGWNAPVNVFVEKKLLPWDGLAKGSIERAV |
| | | | mountain | SKFFSASCIPGATETNLCKQCIGEEEKKCKSSHDE |
| | | | slow frog | PYYGDHGAFRCLQEDKGDVAFLKNTALPDEHSGVY |
| | | | | ELLCPDNTRKPLNKYKECNLGKVPADAVVTRKAGD |
| | | | | KTKDINDFLLEAQKKKCKLFGSPHGKDLMEDDSTT |
| | | | | HLAPLPSEIDAFFFLGVKWYNAMKALTEDVKLPSK |
| | | | | NKVRWCTINKPEMMKCKDWAAVSGGAIACTEASCP |
| | | | | EHCVKQILKGEADAVTLDVQYMYMALMCGLLPAVE |
| | | | | EYPNKDDFHPCQIPGSTIKDFGTKRAVALVKKSNK |
| | | | | DIKWNNLKGKKSCHTHVGDIPGWVIPAGLISNQND |
| | | | | NIDIESFFGESCAPGSDTNSKLCKLCIGDPENPKA |
| | | | | STRCSLSDKEAYYGNEGAFRCLVEKGDVAFVPHTV |
| | | | | VFANTDGKNPAEWAKDLKSEDFEILCLDGSRAPVT |
| | | | | NYRGCNLSGLPPRAIVTREESVSDVVRILINQQSL |
| | | | | YGRNGFEKDMFQMFSSAKGQNLLENDETQCLIEFD |
| | | | | RQPKDIMEDYFGVRYYTAVYSASRSAVPSELIPAC |
| | | | | TFKHC* |
| 3 | MaSxph | Mantella | Golden | MALTFQTALYFTIIGLSFVTSSARDVRWCVISDLE |
| | | aurantiaca | mantella | QKKCNDLVGSCKVSGITLVCVHRSSTENCMTAIRD |
| | | | | EQADAMFLDSGDVYKASMSPYNLKPIIAESYSSHK |
| | | | | ECLKKRQEALGGANVLGQFVPQCDEKGSYQPQQCY |
| | | | | GSTGHCWCVNAIGEEIVGTKTQPGQTQATCERHDQ |
| | | | | TKCLKDRQKALAKKMIGGFVPQCDEKGNFKPRQCH |
| | | | | GSTGYCWCTNANGEKIAATNTPPQQSPPTCERHDL |
| | | | | DSCHYAVAVVKKSSIFQFNQLKGKRSCHSGVSKTD |
| | | | | GWKAPVSVLVDKNLLSWDRAAKESIEKAVSKFFSD |
| | | | | SCIPGATETNLCNQCMGEGGKKCMSSHDEPYYGDH |
| | | | | GAFRCLKEDKGDVAFLKNTVLSDTHSEDYELLCPD |
| | | | | NTRKPLNKYKECNLGKVPADAVVTRKSGDKIKDIN |
| | | | | DFLLEAQKKHCQLFSSAHGKDLMFDDSTTQLSLLP |
| | | | | PEVDVFFFLGVQWLNTMKTLTADVKPPSKNKVRWC |
| | | | | TVNNLEKMKCDDWSAVSGGAIECTEASSPEHCIKQ |
| | | | | IVKGEADAVTLDVQYMYEALKCGLLPAVEEYHNKD |
| | | | | DFGPCKTPGSEIKDFGAKRAVALVKKSNKDINWHN |
| | | | | LKGKKSCHTGVGDQAGWIIPVGLISRQIDNCNIDS |
| | | | | FFSESCAPGSDTTSNFCKLCIGDPSNPMANTKCSL |
| | | | | SDKEAYHGNEGAIRCLVEKGDVAFVHHTAVFENTD |
| | | | | GKNPAEWAANLKSEDFEILCLDGSRAPVSNYMTCR |
| | | | | LSGVPPRAIVTREESVSDVVRILINQQSLFGRNGF |
| | | | | EKDMFQMFSSNKGQNLLFNDGTQCLIEFDRQPRGI |
| | | | | MEDYFGKPYYNAVYNTSKCVLPSELTSACTFKHC* |
| 4 | DtSxph | Dendrobates | Dyeing | MRMTPQVLLWLCLLALSSAAPNIRNVRWCVTSDTE |
| | | tinctorius | poison | EQKCNDLVTSCHVDEILLTCVKKSSTEDCVRAISN |
| | | | dart frog, | GEADAISLDSKDVYKASLDPFNLKPIMTEAYSERE |
| | | | Dyeing | HTPCMRHRQSVLGGKNMKIGAFVPKCDEKGNYVPK |
| | | | dart frog | QCHGSTGYCWCLNENGEEIEGTRTPPGNSGLTCEN |
| | | | | KANKPPCLKERQKLLSAKPSPAVFVPECDEKGNYR |
| | | | | PQQSHVYTWCVDEYGEEVFGSRNFPGKPPKPCEAS |
| | | | | GETACIKERNKVLSVAEPLLGAFLPDCDENGYFSP |
| | | | | LQFHGSTGYSWCVTKNGEEIKGTRTGPGQTPPTCE |
| | | | | VSAPVTLHYAVAVVKKSSSFQLDQLKGKRSCHSAV |
| | | | | GEAAGWVAPLNVLLKKKLLLWEEPEQKSIEKVASE |
| | | | | IFSASCAPGAQEANLCEQCAGQEDQCTRGPGEPYY |
| | | | | GDEGAFRCLRDGKGDVAFVEDTALTGQYSDNFELL |
| | | | | CPDNTRRPLSQYKICNFGRIPRHSVVTRSTGDKMQ |
| | | | | DITEYLLQAQKKECKLFSSTHGKDLLFEDTTSALI |
| | | | | ALPSAMDTFLFLGPDLFNGMKTLNGARPPRVKQQI |
| | | | | RWCPQSKNEHKKCEDWSSVSGGAIKCTEPSSALEC |
| | | | | VKKILKDEADAVNLDTAHTYTALKCGLLPSLDEYR |
| | | | | NKDDLIPCQIPGADYTEFGSYRIVAIVKKKDKDIT |
| | | | | WNNLQGKKSCHTGVGDMMGWNIPVYLISKKTKNCD |
| | | | | LGSYFSQSCAPGSPIDSNLCKLCIGDPQNTEANTK |
| | | | | CSLSDKEAYYGNEGAVRCLVEKGDVAFVPHTAVFE |
| | | | | NTDGKNPALWAKDLKSADFELLCPDGSRAPVSNYK |
| | | | | TCKLSGIANQVTVTRPESVKDVVRITQNQQSLYGR |
| | | | | TGSLKDIFQMFSTSYGQNLLFSDRTQCLVEFDRML |
| | | | | DRDIMDDYFGKPFHKHLIRDNDCFPISALATACSF |
| | | | | HH* |
| 5 | OsSxph | Oophaga | Diablito, | MRMTLQVLLCLCLLVLSSAAPNSRNVRWCVTSDTE |
| | | sylvatica | Little | EQKCNDLVTSCHVDEILLICVKKSSKEDCVRAISN |
| | | | devil | GEADAISLDSKDVYKASLDPFNLKPIMTEAYSESE |
| | | | poison | QTPCMRHRQSVLGGKKMIIGAFVPKCDEKGNYVPK |
| | | | frog | QCHDSTGFCWCLNENGEEIEGTRTPPGNSGLTCEN |
| | | | | KANKPPCLKERQKLLSAKPSPAVFVPECDEKGNYR |
| | | | | PEQSHVYSWCVDEYGEEVLGSRTFPGKPPKPCEAS |
| | | | | GETPCIKERNKVLSAAEPLPGAFVPDCDEKGYFSP |
| | | | | LQFHGSTGHSWCVTKNGEEIKGTRTRPGQTPPTCD |
| | | | | VPAPATLHYAVAVVKKSSSFQLDQLKGKRSCHSAV |
| | | | | GEAAGWVAPLNVLLKKKFLLWEEPEQKSTEKVASE |
| | | | | IFSASCAAGAQEANLCEQCAGQEDKCTRGPGEPYY |
| | | | | GDEGAIRCLRDDKGDVAFVEDTALTGQYSDNFELL |
| | | | | CPDNTRRPLSQYKICNFGSVPRHAVVTRSTGDKMK |
| | | | | EITEYLLQAQKKECKLFSSTHGKDLLFEDTTSALI |
| | | | | ALPSGMDTFLFLGPDLFNGMKTLNGAHPPRVKQKI |
| | | | | RWCPQNKNEKKKCDDWSSVSGGSIKCTEPSSALEC |
| | | | | IKMILKDEADAVNLDAEHAYTALKCGLLPSLEEYR |
| | | | | NKDDLIVCQIPGAEYSEFGSYRIVALVKKTDKDIT |
| | | | | WNNLQGKKSCHTHVGDMIGWNIPVYLISKKTKNCD |
| | | | | LGSYFSQSCAPGSPIDSNLCKLCIGDPQNTEANTK |
| | | | | CSLSDKEAYYGNEGAVRCLVETGDVAFVPHTAVFE |
| | | | | NTDGKNPALWAKDLKSTDFELLCPDGSRAPVSDYK |
| | | | | KCKLSGIANHVTVTRPESVKDVVRITQNQESLYGR |
| | | | | TGSQKDIFQMFSSSYGQNLLFNDRTQCLFEFDRML |
| | | | | DRDIMDDYLGKPFHKHLMSDNDCLPKSALATACSF |
| | | | | HH* |
| 6 | RiSxph | Ranitomeya | Mimic | MRRSSYAASQEMRMTLRVLLCLCLWALSSAAPNTR |
| | | imitator | poison | NVRWCVTSDTEEQKCNDLVTSCHVDEILLICVKKS |
| | | | frog, | STEDCVRAISNGEADAISLDSKDVYKASLDPFNLK |
| | | | Poison | PIMTEAYSESEHTPCMRHRQSVLGGKIMKIGAFVP |
| | | | arrow | KCDEKGNYVPKQCHGSTGYCWCLNENGEEIEGTRT |
| | | | frog | PPGNPGLTCENKANKPPCLKERQKLLSAKPSPAVF |
| | | | | VPECDEKGNYRPQQSHVYSWCVDEYGEEVFGSRSF |
| | | | | PGKPPKPCEASGETPCIKERNKVLSAAEPLLGAFV |
| | | | | PDCDEKGYFSPLQSHGSTGYSWCVTKNGDEIKGTR |
| | | | | TGPGQSPPTCEVPAPVTLHYAVAVVKKSSSFHLDQ |
| | | | | LKGKRSCHSAVGEAAGWVAPLNVLLKKKLLLWEEP |
| | | | | EPKTIEKVASEIFSASCAPGAQEANLCEQCAGQED |
| | | | | KCTRGPGEPYYGDEGAFRCLREDKGDVAFVEDTAL |
| | | | | TGQYSDNFELLCPDNTRRPLSQYKICNFGRIPRHA |
| | | | | VVTRSSGDKMKDITEYLLQAQKKECKLFSSTHGKD |
| | | | | LLFEDTTSALIALPSAMDTFLFLGPDLFNGMKTLN |
| | | | | GARPPQVNQQIRWCPQSKNEKKKCDDWSSVSGGAI |
| | | | | KCTEPSSGLECIKMILKGEAEAVNLDIEHSYIALK |
| | | | | CGLLPSVDEYRNKDDLIPCQIPGSEYSEFGSYRIV |
| | | | | ALVKKTDKDLTWNNLQGKKSCHTRAGDMIGWNIPV |
| | | | | YLISKKTKNCDLGSYFSQSCAPGSPIDSNLCKLCI |
| | | | | GDPQNPQANTKCSISDKEAYYGHEGAIRCLVEKGD |
| | | | | VAFVPHTAIFENTDGKNPALWAKDLKSTDFELLCP |
| | | | | DGSRAPVNDYKKCKLSGIANQIAITRPESVKDVVR |
| | | | | IIQNQQSLYGRTGSQKDIFQMFSSSYGQNLLFSDR |
| | | | | TQCLVEFDRMLDRDIMDDYFGKPFHKHLLRDNDCL |
| | | | | PKSALASACSFHH* |
| 7 | PtSxph | Phyllobates | Golden | MRMTTAFQVLLCVCLLALSSAAPNARNVRWCVTSD |
| | | terribilis | dart frog, | AEEQKCNDLVTSCHVDEILLTCVKKYSTEDCVRAI |
| | | | Golden | SNGEADAISLDSKDVYKASLHPFNLKPIVTEAYSE |
| | | | poison | KEHTPCMRHRQSVLGGKKIKIGAFVPKCDEKGNYV |
| | | | frog, | PKQCHGSTGYCWCLNENGEEIEGTRTPPGTKHLTC |
| | | | Golden | EDTADKPPCLKKRQKLLSAKPSPAVFVPECDEKGN |
| | | | poison | YRPEQSHVYTWCVDEYGEEVFGSRNFPGKPPKPCV |
| | | | arrow | ASDEPLCIKERNKVLGTTEPLLGAFVPDCDEKGYF |
| | | | frog | SPLQFHGSTGYSWCVTENGVEIKGTRTGPGQTPPT |
| | | | | CEVPAPMTLNYAVAVVKKSSSFHLDQLKGKRSCHS |
| | | | | AVGEAAGWVAPLNVLLKKKLLSWGGPEQKSIEKVA |
| | | | | SEIFSASCAPGAQEANLCEQCAGQEDKCTRGPGEP |
| | | | | YYGDEGAFRCLRDDKGDVAFVEDTVLSGQYSDNFE |
| | | | | LLCPDNTRRPLSQYKICNFGRIPRHAVVTRTTGDK |
| | | | | MKDITEYLVQGQKKECKLFSSTHGKDLLFEDTTTA |
| | | | | LITLPSAMDTFFFLGSELFKGMKTLEGTRPPRVHR |
| | | | | EIRWCPQNKNEKKKCDDWSSVSGGAIKCTEPSSAL |
| | | | | ECIEMILKGEAEAVNLDAEHAYTALKCGLLPSVEE |
| | | | | YRNKDDLIPCQVPGAEYSEFGGNRIVALVKKTDKD |
| | | | | ITWNDIQGKKSCHTHVGDMIGWNIPIYLIYKKTKN |
| | | | | CDIGSFFSQSCAPGSTIDSNLCKLCIGDPQNPEAN |
| | | | | TKCSLSDKEAYYGNEGAIRCLVEKGDVAFVPHTAV |
| | | | | YENTDGKNPALWAKDLKSTDFELLCLDGSRAPVSD |
| | | | | YKKCKLSGIVNQVTVTRPESVKDVVRIIHNQESLY |
| | | | | GRTGFQKDIFQMFSSSYGENLLFSDRTQCLFEFDR |
| | | | | MLDRDIMEDYLGKPFHKYVMRDNECLPKSALATAC |
| | | | | SFHH* |
| 8 | EtSxph | Epipedobates | Phantasmal | MVSSRKLRMTSAFQLLLCLCLMVLSFAAPNARNVR |
| | | tricolor | poison | WCVTSDAEEHKCNDLVNSCHVNDILLKCVKKSSTE |
| | | | frog, | DCVRAISNGEADTISLDSKDVYKASLHPFNLKPIM |
| | | | Phantasmal | TEGYSEREHTPCMRHRQSVLEGKKMKIGAFVPKCD |
| | | | poison- | EKGNYAPKQCHGSSGYCWCLNENGEEIEGTRTPPG |
| | | | arrow | TKSLTCEDAANKPPCLKERQKLLSSKPAPTVFVPE |
| | | | frog | CDEKGNYRPQQCHIYCWCVDEYGEEVLFSRSFPGK |
| | | | | PPKKCEPSGETLCLKQRNKVLSATEPLLGTFVPEC |
| | | | | DDKGYFTPQQFHGSTGYSWCVTKNGDEIPGTRTGP |
| | | | | GQTPHICEVKAPVTLHYAVAVVKKSSSFQLDELKG |
| | | | | KRSCHSAVGEAAGWVAPLKILLKKKLLSLEGAEVK |
| | | | | SIEKAASEIFSASCAPGAHEAKLCEQCAGQEGKCH |
| | | | | RGPGEPYYGDEGAFRCLRDNKGDVAFVEDTALSGQ |
| | | | | YSDNYELLCPDNTRRPLSQYKICNFGRVPRHAVVT |
| | | | | RSSGDKMKDITEYLLEAQKKECKLFSSTHGKDLLF |
| | | | | EDTTTGLIALPSAMDTFLFLGPELFHGMKTLDGAH |
| | | | | PPSSQEIRWCPLTKNEKKKCDDWSSVSGGAIKCTE |
| | | | | PSSGQKCIEKILKGEADVVTLGVEHAYTALKCGLH |
| | | | | ISFEEYRNKDDLIPCQVLEAEYTEFGSYRIVAVVK |
| | | | | KTDKDITWNNLQGKKSCHTSVNDMVGWKIPHALLY |
| | | | | KKTKSCDFGSYFSKSCAPGSAIDSNLCKLCIGDPQ |
| | | | | NTKSNTKCSLSDKEAYYGNDGAIRCLVEKGDVAFV |
| | | | | PHTAIFENTDGKNPALWAKDLKSTDFELLCLDGSR |
| | | | | APVSDYKTCKLSGIANQVTVNRPERQADVLRIILN |
| | | | | QQSLYGRTGSQKDIFQMFSSSYGQNLLFNDRTQCL |
| | | | | FVFDRMLDRDIVDDYIGKAMQRDVLRDNECFPQTA |
| | | | | LTNACLFHH* |
| 9 | AfSxph | Allobates | Brilliant- | MRMTPALLGLLCLCLLVLSSAAPNSRTVRWCVTSD |
| | | femoralis | thighed | TEQQKCNDLVNSCHVDEIVLSCVKKSNTEDCVRAI |
| | | | poison | STGEADAISLDSKDVYKASLDPFHLKPIMTEGYSE |
| | | | frog, | RDHTPCMKHRQSVLGGKQLIIGAFVPKCDEKGNYA |
| | | | Brilliant- | PKQCHGSSGYCWCLNENGEEIKGTRSRPGTKVLTC |
| | | | thighed | EDAANKPPCLKERQKLLSENPSPTVFVPECDEKGN |
| | | | poison | YRPRQCHDYCWCVDEYGEEIFGSRTFPGKPPKACE |
| | | | arrow | ASGETLCIKERNKVLSTAEPLRGAFLPECDEKGYF |
| | | | frog | SPMQCHSSTGHCWCTTKDGEEIEGTRTGPGQSRPT |
| | | | | CDIPAPGTVHYAVAVVKKSSNFQLDGLKGKRSCHS |
| | | | | AVGESGGWVAPLNIFLKKNLLSWEGPEQKSIEKAA |
| | | | | SEFFSASCAPGAQESTLCEQCAGQEDKCKRGPGEP |
| | | | | YYGDEGAIKCLKDDKGDVAFVEDTILSGQYSNDFE |
| | | | | LLCSDNTRRPLSQYKMCNFGKIPRHAVVTRSSGDK |
| | | | | MEDITEYLLKAQKKECKLFGSAVGKDLLFEDTTTS |
| | | | | LIVLPSAMDTFLYLGPELFSGMKTLEGAHPPSNQE |
| | | | | IRWCPQSKNEKKKCDDWSSVSGGAIKCTEPSSALE |
| | | | | CIEKILKGEAEAVTLDGEHAYTALKCGLVPSLDEY |
| | | | | RNKDDLVPCQIPGAEYSEFGSYRIVALVKKTDKDI |
| | | | | TWDNLQGKKSCHTSVGDMMGWTVPISLIYKKTKNC |
| | | | | DIGSYFSQSCAPGSAIDSNLCELCIGDPGNPTANT |
| | | | | KCSQNDKEAYYGNEGAIRCLVEKGDVTFVPHTAVY |
| | | | | ENTDGKNPALWAKDLKSTDFELLCPDGSRAPVSDY |
| | | | | KRCKLSGIANQITVTRSESLKDVVRIILNQQSLYG |
| | | | | RNGFQKDIFQLFSSVHGQNLLFSDRTQCLVEFDRM |
| | | | | LDRDIMDDYFGKPFHKTVFRDNDCLPKSALATACS |
| | | | | FHH* |
| 10 | RmSxph | Rhinella | Marine | MTPAFQVLLCLCLLGMSYAAPQARNVRWCVTSDAE |
| | | marina | toad, | EEKCNDLVNTCTVKEILLICVKKSSTEDCIRAISA |
| | | (Bufo | South | GEADAISLDSEDIYQASLKPFHLKPIMTETYASKK |
| | | marinus) | American | NIKLNQSPCQKEHQHLLEGGPKCDETESTPCLRHR |
| | | | cane | QRVLGARKPQIGEFVPECDEKGNYFPKQCYGTTGY |
| | | | toad, | CWCVDEHGDEIPVGRAKQGKVNISCEYAASEKPCM |
| | | | Cane | KEQRKALSGGQPLRGAFIPNCDEKGNYSPKQCHGS |
| | | | toad, | TGYCWCVDENGAEISGSRTPPGQQVPTCGSYVGAT |
| | | | Giant | CIKDRYKVLGAGKPLPGAFVPDCDEKGDYRPQQCH |
| | | | toad | GSTGHCWCVSKDGVEIQGTRAAPGQSPPTCEDPDL |
| | | | | VKSHYAVAVVKKSSTLQFNHLKGKRSCHSAVGKTA |
| | | | | GWIAPLYALYKKHFLLWDGSEEKSFEQAASEFFSA |
| | | | | SCAPGAKEENLCKQCAGQEDNCKSSPGEPYYGDEG |
| | | | | ALRCLREDKGDVAFVEHTALSGKDSDNFELLCPDN |
| | | | | TRSPVSEHKDCNFGKVPRHAVVTRDTGDKSKDIIE |
| | | | | YLLEAQKKECKLFSSTCGKDLMSGDTTTNLIALPS |
| | | | | GMDAFLFLGPELFNAMKTLHGAPLPSKNEILWCTQ |
| | | | | NKNEKTKCDDWSAVSGGAIKCTEPLSADQCIEKIL |
| | | | | KREADAVTLHTEHMYTALKCGLVPAVEGYYNKDDF |
| | | | | TPCKTDGDFYKDFGGTYVVALVKKSDKDITWNNLK |
| | | | | GKKSCHTGVGHWTGWMIPVSQISKQTKNCDIGSYF |
| | | | | SQSCAPGSAIDSNLCELCIGDQQNTKAQTQCSPSD |
| | | | | KEAYYGGAGAIRCLVEKGDVAFVTDRDVFKNTDGN |
| | | | | NPALWAKDLKSTDFELLCLDGSRAPVTDYKNCNLP |
| | | | | GNPNLSVVSREENVNDVVRIFLNQQSLYGRNGSQK |
| | | | | DKFQMFSSYGENLLFSDDAQCMIEFDMIKGRDLID |
| | | | | DVLGKHFSATMLRNNPCLPKSELRTACTFRAEKI |
| 11 | BbSxph | Bufo | Common | MKHSSSSAQKEVRMTPAFQVLLCLCLLGMSYAAPQ |
| | | | toad, | ARNVRWCVTSDAEEEKCNDLVNSCPVKEILLICVK |
| | | | Caucasian | KSSIEDCMRAISAGEADAISLDSENIYKASLKPFH |
| | | | toad | LKPIMTETYISKKDIKLTQGPCQKEHQHLLEGRQH |
| | | | | KCEETESTPCLRHRQRVLGAKKPHIGEFMPECDEK |
| | | | | GNYFPKQCYGTTGYCWCVDENGAERHVGRAKQGKV |
| | | | | NISCEYTESQKPCMKERRKVLSGGQPLPGAFIPDC |
| | | | | DEEGNYNPKQCHGSTGYCWCVDENGAEISGSRMPT |
| | | | | GQSDPTCGTYAGATCIKDRYKVLGAGEALRGAFVP |
| | | | | DCDEKGDYRSKQCHGSTGYCWCVSKYGVEIQGTRT |
| | | | | APGQSPTTCEIPGGEKPCMKERRKSLSGGQPLPGA |
| | | | | FMPDCDEKGNYSPKQCHGSTGYCWCVNENGKEISG |
| | | | | SRTPPGQQVPTCVASVLSAETSCIKERQKVLGAVK |
| | | | | PILGAFVPDCDEKGEYRPKQCHGSTGYCWCVSKDG |
| | | | | KEIQGTRAAPGQSPPTCQDTGEKLVLSAETSCIKE |
| | | | | RQKVLGAVKPILGAFVPDCDEKGEYRPKQCHGSTG |
| | | | | YCWCVSKDGKEIQGTRAARGQSPPTCQDTGEKLVL |
| | | | | SAETSCIKELQKVLGAVKPMVGAFVPDCDEKGEYR |
| | | | | PKQCHGSTGYCWCVSKDGKEIQGTRVAPGQSPPTC |
| | | | | QDTVLSAETSCIKERQKVLGAVKPILGAFLPDCDE |
| | | | | KGEYRPKQCHGSTGYCWCVSKDGKEIQGTRAAPGQ |
| | | | | SPPTCQDTGEKLVLSAETSCIKERQKVLGAVKPIV |
| | | | | GAFVPDCDEKGEYRPKQCHGSTGHCWCVSKDGKEI |
| | | | | QGTRVAPGQSPPTCQDTVLSAETSCIKERQKVLGA |
| | | | | VKPILGAFLPDCDEKGEYRPKQCHGSTGYCWCVSK |
| | | | | DGKEIQGTRAAPGQSPPTCQDTVLSAETSCIKERQ |
| | | | | KVLGAVKPILGAFLPDCDEKGEYRPKQCHGSTGYC |
| | | | | WCVSKDGKEIQGTRAAPGQSPPTCQDTGEKLVLSA |
| | | | | ETSCIKERQKVLGAVKPIVGAFVPDCDEKGEYRPK |
| | | | | QCHGSTGHCWCVSKDGKEIQGTRVAPGQSPPTCQD |
| | | | | TVLSAETSCIKERQKVLGAVKPILGAFLPDCDEKG |
| | | | | EYRPKQCHGSTGYCWCVSKDGKEIQGTRAAPGQSP |
| | | | | PTCQDTGEKLVLSAETSCIKERQKVLGAVKPIVGA |
| | | | | FVPDCDEKGEYRPKQCHGSTGHCWCVSKDGKEIQG |
| | | | | TRVAPGQSPPTCQDTVLSAETSCIKERQKVLGAVK |
| | | | | PILGAFVPDCDEKGEYRPKQCHGSTGYCWCVSKDG |
| | | | | KEIQGTRAAPGQSPPTCQDTGEKLVLSAETSCIKE |
| | | | | RQKVLGAVKPIVGAFVPDCDEKGEYRPKQCHGSTG |
| | | | | HCWCVSKDGKEIQGTRVAPGQSPPTCQDTDPVTCH |
| | | | | YAVAVVKKSSTLQFNQLKGKRSCHSAVGKTAGWIA |
| | | | | PLYKLYKKNLLLWEKPEEKSFEKAASEFFSVSCAP |
| | | | | GAKEENLCKQCAGKEDKCKRSPGELYYGDEGALRC |
| | | | | LREDKGDVAFLEDIALSGQDLDNFELLCPDNTKSP |
| | | | | LSEQKHCHFGKVPSHAVVTRSTGDKSKDIIEYLLE |
| | | | | AQKKGCKLFSSTHGKDLMFEHTTTNVIALPSAMNT |
| | | | | FLFLGPELFDAMKTLHGTPLPSKNEVRWCTQNQNE |
| | | | | KTKCDDWSSVSGGAIKCTEPSSVQQCIEKILKHEA |
| | | | | DAVTLSAEHMYTALKCGLVPAVDEYHNKDDFAPCR |
| | | | | TLGDIYTDFGTPRAVALVKKSNKDITWNNLKGKKS |
| | | | | CHTGVGHMAGWVIPLSLISKQTNNCDLGSYFSQSC |
| | | | | APGSAIDSNLCKLCIGDPQNTKAQTQCSPSDKEAY |
| | | | | YDSAGAIRCLVEKGDVAFLPHTAVFENTDGNNPAL |
| | | | | WAKDLKSTDFELLCPDGSRAPVTDYKNCKLLSISS |
| | | | | PSVVTREESVSDVVRIVLSQQSLFGRKGFEKDMFQ |
| | | | | MFSSSNGKNLLFSDGTQCLLEFDRIIGRDIMEDYF |
| | | | | GKPFHTAVHRDNQCLPTSELASACAFHHC* |
| 12 | BgSxph | Bufo | Asiatic | MKHSSSSAQKEVRMTPAFQVLLCLCLLGMSYAAPQ |
| | | gargarizans | toad | ARNVRWCVTSNAEEKKCNDLVNSCTVKEIVLICVK |
| | | | | KSSTEDCMRAISAGEADAISLDSENIYKASLNPFD |
| | | | | LKPIMTETYPSQKEIKVNQGPCQKERQRQRERGRP |
| | | | | LLGAFEPKCDEKGNYQPKQCHGSTGYCWCVNEEGK |
| | | | | TIDGTKTPPGQKSVTCEDHQQSTPCLRHRQSVLGA |
| | | | | NKPQIGAFVPDCDEKGNYSPKQCFGSTGYCWCVDE |
| | | | | HGDEIEGVRAKQGKVNITCEYTGGEKPCMKERRKS |
| | | | | LSGGQPLPGAFMPDCDEKGNYSPKQCHGSTGYCWC |
| | | | | VNENGKEISGSRTPPGQQVPTCGASVLSAETSCIK |
| | | | | ERQKVLGAATPILGAFVPD |
| | | | | CDAKGDYRPKQCHGSTGHCWCVSKDGKEIQGTRTA |
| | | | | PGQTPPTCEIPGGEKPCMKERRKSLSGGQPLPGAF |
| | | | | MPDCDEKGNYSPKQCHGSTGYCWCVNENGKEISGS |
| | | | | RTPPGQQVPTCGASVLSAETSCIKERQKVLGAEKP |
| | | | | ILGAFVPDCDEKGDYRPKQCHSSTGHCWCVSKDGK |
| | | | | EIQGTRTAPGQTPPTCEIPGGEKPCMKERQKSLSG |
| | | | | GQPLPGAFMPDCDEKGNYSPKQCHGSTGYCWCVNE |
| | | | | NGKEISGSRTPPGQQVPTCGASVLSADTSCIKERQ |
| | | | | KVLGAEKPILGAFEPDCDEKGDYRPKQCHGSTGHC |
| | | | | WCVSKDGKEIQGTRTARGQSPPTCEIPVLSAETSC |
| | | | | IKERQKVLGAATPILGAFVPDCDEKGDYRPKQCHG |
| | | | | STGHCWCVSKDGKEIQGTRAAPGQTPPTCEDRVLS |
| | | | | AETSCIKERQKVLGAATPILGAFVPDCDEKGEYRP |
| | | | | KQCHGSTGHCWCVSKDGKEIQGTRAGPGQTPPTCE |
| | | | | DTAETSCIKERQKVLGAAKPILGAFVPDCDEKGEY |
| | | | | RPKQCHGSTGHCWCVSKDGKEIQGTRAAPRQSPPT |
| | | | | CEIPVLSAETSCIKEQQKVRAGKPILGAFVPDCDE |
| | | | | KGDYRPKQCHGSTGHCWCVSKDGKEIQGTRAACGQ |
| | | | | SPPTCEIPAETSCIKERQKVLGAEKPILGAFVPDC |
| | | | | DEKGEYRPKQCHGSTGHCWCVSKDGKEIQGTRAGP |
| | | | | GQSPPTCEDTVLSAETSCIKERQKVLGAATPILGA |
| | | | | FVPDCDEKGDYRPKQCHGSTGHCWCVSKDGKEIQG |
| | | | | TRAGPGQSPPTCEIPAEKSCIKERQKVRSPRKPIL |
| | | | | GAFVPDCDEKGDYRPKQCHSSTGHCWCVSKDGKEI |
| | | | | QGTRAARGQSPPTCEDPDPVTCHYAVAVVKKSSTL |
| | | | | QFNQLKGKRSCHSAVGKTASWIAPLYKLYKKNLLL |
| | | | | WEKPEEKSFEKAASEFFSVSCAPGAKEENLCKQCA |
| | | | | GKEEKCKRSPGEPYYGDEGALRCLRDDKGDVAFVE |
| | | | | HTALSGQDLDNFELLCPDNTRSPLSEHKHCHFGKV |
| | | | | PRHAVVTKSTGDKSKDIIEYLLEAQKKGCKLFSST |
| | | | | HGKDLIFEHTTKNLIALPSAMNTFLFLGPELFDAM |
| | | | | KTLHGAPLPSKNEVRWCTQNKKEKTKCDDWSSVSG |
| | | | | GAIKCTEPSSVQQCIEKILKHEADAVTLSAEHMYT |
| | | | | ALKCGLVPAIDEYHNKDDLAPCKTLGDIYTDFGTP |
| | | | | RAVALVKKSDKGITWNNLKGKKSCHTGVGHMAGWV |
| | | | | IPLSLISKQTNNCDMGSYFSQSCAPGSAIDSNLCK |
| | | | | LCIGDPQNTKAQTKCSPSDKEAYFGSAGAIRCLVE |
| | | | | KGDVAFLPHTAVIENTDGN |
| 13 | EpSxph | Engystomops | Túngara | NPALWAKDLKSTDFELLCPDGSRAPVTDYKNCKLL |
| | | pustulosus | frog | SISSPSVVTREESVSDVVRIVLNQQSLFGQKGFEK |
| | | | | DIFQMFSSSNGKNLLFSDGTQCLLEFDRIIGRDIM |
| | | | | EDYFGKRFHTAVHRDNQCLPTSEFASACAFHHC*M |
| | | | | TAISHGEADAISLHGENIYKASFKPFNLIPIITES |
| | | | | YHYQKGGRPLLGAFVPKCDEKGNYSPTQCHGGTGY |
| | | | | CWCVTAEGKEIDGTKKPPGESPTCGEETKKTPCLN |
| | | | | KREKALSGGQPLLGAFAPDCDEEGNFKPRQCYGST |
| | | | | GYCWCVDDNGVEISATRTPPGEKPPTCGATDPVTC |
| | | | | HYAVAVVKKSSTFQFNQLKGKRSCHSAVGESAGWV |
| | | | | APMSALLDKKFLLWEGPAKKSFEKAASEFFSASCA |
| | | | | PGAKEENLCKQCAGQQDKCKQSPGEPYYGDEGAFR |
| | | | | CLREDKGDVAFVEHTVLSGQYSDNYELLCPDKTRK |
| | | | | PLSQYEHCHFGKVPRPAVVTRHTGRKTKDITDYLL |
| | | | | EAQKKECKLFSSTHGKNPPDTTTTLTSLPLAMDTF |
| | | | | LFLGPELFNAMKRMHGEPLPSNKEVRWCPQSSDEK |
| | | | | KKCDDWSAVSGGAIKCTEPFTTLQCIEKVLKGEAE |
| | | | | AVTLNVENMYTALKCGLVPIVEEYHNKDDFNPCQS |
| | | | | RGAQIKDFGTVKAVAVVKKRDKDINWNNLKGKKSC |
| | | | | HTGVGHLAGWVVPVSLINKQSRTCDVESYFNQSCA |
| | | | | PGSTTKSNLCKLCIGDPQNTKAKTKCSPNDKEAYY |
| | | | | GNEGAIRCLAEKGDVAFVPHTAVFENTDGKNPALW |
| | | | | AKDLKSTDFELLCPDGSRAPVSNYKNCKLAGIPPR |
| | | | | AVVTRKDSASDVTKIIVNQQSLFGRKGFQKDIFQM |
| | | | | FSSSNGQNLLFSDNTQCLIEFDRMIEKDIMEDYFG |
| | | | | KTYYTAVHSDNQCFPPSALASACSFHHC* |
| 14 | EcSxph | Eleutherodactylus | Puerto | MTPAFQLLLCLCLLGLSYTAPQGRNVRWCVTSDIE |
| | | coqui | Rican | DKKCNDLANSCTVNEIHLKCVKKSSTLDCVKTISD |
| | | | coqui | GEADAISLDGEDIYKASLNPFNLKPIMVESHYHRA |
| | | | | KNLPCLKERQEALGGGHPLPGAFVPNCDEKGNYNP |
| | | | | KQCHGSTGYCWCVNKNGQKINGTETPPGQPSPTCE |
| | | | | EEKSTPCLKHRQRLLGGNKPKIGAFVPKCDEKGDY |
| | | | | IPKQCHGSTGQCWCVNTDGEEIAGTRTGPGKAPPT |
| | | | | CEDAEPVTCHYAVAVVKKSSTLQFDQLKGKRSCHS |
| | | | | AVGTAAGWAAPLNALMKRNLLVWKGPKEKTIEKAA |
| | | | | SEFFLSSCAPGAKETNLCKQCAGKEANCKHSSEEP |
| | | | | YYGDEGAFKCLRDDKGDVA |
| 15 | RtSxph | Rana | European | FVENSVLSEEHTDSFELLCPDNTRKQLSQYKDCNF |
| | | temporaria | common | GKVPRHAVVTRSSGDKIKDITEYLLGAQKKECELF |
| | | | frog, | SSTHGKRFLFEDTTTNLIALPSAMDTFLFLGPELF |
| | | | European | DAMKALHGEHPPSKNEVRWCTLSEKEKLKCDDWSS |
| | | | common | VSGGAIKCTEPSLAEECIEKVLKGEADVVSLSKKH |
| | | | brown | MYAALTCGLIPAFEEYHNKDDFGPCQTPGTQYTDF |
| | | | frog, | GMTSNVALVKKKDQDITWNNLKGKKSCHPEVDDSA |
| | | | European | GWVDPLILIKKQTKSCDLGSFFSKSCAPGSAIDSN |
| | | | grass frog | LCELCIGDSQNSKANTKCSRSDKEAYYGNDGAIRQ |
| | | | | KGDVAFVPHFAVSDNTDGKNPALWAKDLKSSDFEL |
| | | | | LCPDGSRAPVSDHKKCKLGGASIQTVVCREDRVSD |
| | | | | VVRIIQNQQSLYGRKGFQKDIFQIFSSRHGRNILF |
| | | | | HDDTQCLVDFDRVKEIDIMYDFFGKEHYDSIDFCL |
| | | | | LIPSLIRRLGNRDLVIEMSLLVVFIPPS*MAPTFK |
| | | | | AALFFTIISLSFAAPNAKQVRWCAISDLEQKKCND |
| | | | | LVGSCNVQDITLVCVLRTSTEDCLTAIKDGQADAM |
| | | | | FLDSGDVYEASKDPYNLKPIIAEPYSSHKDLQKCL |
| | | | | KEREQALAKKMIGHHIPQCDEKGNYQPQQCHGSTG |
| | | | | QCWCVNGMGEKISGTNTPPGQTRATCERHELPKCL |
| | | | | KERQVALGGDEIVLGRFVPQCDEKGNYEPQQFHGS |
| | | | | TGYSWCVNEIGEEIAGTKTPPGKIPVTCEKHDLVT |
| | | | | TCHYAVAMVKKSSTFQFNQLKGKRSCHSGVSKTDG |
| | | | | WKALVTVLVEKKLLSWDGPAKESIQQAMSKFFSVS |
| | | | | CIPGATQTNLCKQCKGEEGKNCKNSHDEPYYGNHG |
| | | | | ASRCLKEDKGDVAFLRSTALSDEDSEAYELLCPDN |
| | | | | TRKPLNKYKECNLGTVPVGAVVTRKIGDKTVDINN |
| | | | | FLMEAQKKQCKLFSSAHGKDLMFDDSTLQLALLSS |
| | | | | GVDAFLYLGVKLFNTMKALTEDAHLSSKNKVRWCT |
| | | | | INKLEKMKCDDWSAVSGGAIECTEASCPKCCIKQI |
| | | | | LKGEADAVKLEVQYMYEALTCGLLPAVEEYHNKDD |
| | | | | FGPCKTPGSPYTDFGSPRAVALVKKSNKDINWNNI |
| | | | | KGKKACLTGVGDIAGWVIPVSLIRRQNDNCDIDSF |
| | | | | FGESCAPGSDTKSNLCKLCIGDPKNSVANTKCSLG |
| | | | | DNEAYHGNKGAFRCLVEKGDVAFVPHTVVFENTDG |
| | | | | KNPAEWAKKLKSEDFELLCLDGSRSPVSNYKSCKL |
| | | | | SGIPPPAIVTREQSVTDVVRIVTNQQSLYGRKGFE |
| | | | | KDIFQLFSSNKGKNLLFNDNTQCLIEFDRQPKDIM |
| | | | | EDYFGKSYYTTVYGASRSAMSSELISACTVKHC* |

* End of sequence

TABLE 4
RcSxph and NpSxph Mutations

| | NpSxph Mutations in Nanorana |
|---|---|
| RcSxph Mutations in Rana | parkeri (High Himalaya frog, |
| (Lithobates) catesbeianus | Xizang Plateau frog, Parker's |
| (American bullfrog, Bullfrog) | slow frog, mountain slow frog) |
| I782A/Y558A | I559Y |
| Y558I | |
| Y558A | |
| I782A | |
| D785N | |
| K789A | |
| T563A | |
| Y558F | |
| Q787E | |
| Y795A | |
| Q787A | |
| F784Y | |
| I782F | |
| F561A | |
| D785A | |
| F784L | |
| P727A | |
| E540D | |
| F784A | |
| D794E | |
| F784C | |
| D794N | |
| F784S | |
| E540Q | |
| D794A | |
| E540A | |

The saxiphilins of the disclosure may be of any length including from about 10 to about 1000 amino acids residues in length, from about 10 to about 300 amino acids in the length, from about 10 to about 200 amino acids in the length, from about 10 to about 400 amino acids in the length, from about 10 to about 100 amino acids in the length, from about 5 to about 60 amino acids in the length, from about 5 to about 70 amino acids in the length, from about 5 to about 80 amino acids in the length, from about 5 to about 90 amino acids in the length, from about 5 to about 100 amino acids in the length, from about 7 to about 30 amino acids in the length, from about 100 to about 500 amino acids in the length, from about 100 to about 600 amino acids in the length, or any positive integer in between those values. In some embodiments, the saxiphilin is no more than 1200, 1100, 1000, 900, 800, 700, 600, 500, 400, 300, 200, 100, 90, 80, 70 or sixty amino acids in length, but may comprise any one or plurality of probes which are detectable after the toxins disclosed herein catalyze the production of reaction products upon contact with saxiphilins.

In some embodiments, the one or plurality of probes, stains and/or substrates comprised in the system of the disclosure are immobilized, adsorbed, bound, or otherwise associated with a reaction vessel in a solid support or a bead, such as a magnetic bead. Solid supports can be tissue culture plates, plastic or polystyrene multiwall plates or other plastic element with one or a plurality of reaction vessels. Toxins contained in a sample can be contacted with the one or more saxiphilins within one or a plurality of reaction vessels on the plastic element for a time period sufficient to catalyze a reaction between the toxin and saxiphilin. In some embodiments, saxiphilins may be encapsulated by or associated with nanodroplets. In some embodiments, reaction products such as cleavage product can be detected in solution or within the reaction vessel after exposure of the reaction vessel to one or a plurality of chemical stimuli for a chemiluminescent probe, visible or non-visible light that is capable of activated the electronic state of a fluorescent probe, or exposure to an antibody specific to the enzyme or substrate. In some embodiments, the saxiphilins disclosed herein can be bound to the surface of the solid support where one or more of FRET analysis, Raman spectroscopy, mass spectroscopy, fluorescent microscopy or absorbance of light may be performed after the enzymatic reaction is complete.

Any type of solid support typically used by one of ordinary skill in the art may be used. In some embodiments, the solid support is a chip. In some embodiments, the solid support is a slide. In some embodiments, the solid support is a petri dish or polystyrene plate. In some embodiments, the solid support is a multiwell plate, including but not limited to, 12-well, 24-well, 36-well, 48-well, 96-well, 192-well, and 384-well plate.

In some embodiments, the one or plurality of probes, stains and/or saxiphilins comprised in the system of the disclosure is in an amount that is enzymatically effective. In some embodiments, any of the systems disclosed above may further comprise one or a plurality of saxiphilins, probes and/or stains specific for toxin in an amount that is enzymatically effective. In some embodiments, the enzymatically effective amount of saxiphilin specific for a toxin comprises from about 0.01 to about 100 μmol/L. In some embodiments, the enzymatically effective amount of saxiphilin specific for a toxin comprises from about 0.1 to about 50 μmol/L. In some embodiments, the enzymatically effective amount of saxiphilin specific for a toxin comprises from about 1 to about 40 μmol/L. In some embodiments, the enzymatically effective amount of saxiphilin specific for a toxin comprises from about 3 to about 35 μmol/L. In some embodiments, the enzymatically effective amount of saxiphilin specific for a toxin comprises from about 5 to about 30 μmol/L. In some embodiments, the enzymatically effective amount of saxiphilin specific for a toxin comprises from about 10 to about 20 μmol/L. In some embodiments, the enzymatically effective amount of saxiphilin specific for a toxin comprises about 0.1 μmol/L. In some embodiments, the enzymatically effective amount of saxiphilin specific for a toxin comprises about 0.5 μmol/L. In some embodiments, the enzymatically effective amount of saxiphilin specific for a toxin comprises about 1 μmol/L. In some embodiments, the enzymatically effective amount of saxiphilin specific for a toxin comprises about 5 μmol/L. In some embodiments, the enzymatically effective amount of saxiphilin specific for a toxin comprises about 10 μmol/L. In some embodiments, the enzymatically effective amount of saxiphilin specific for a toxin comprises about 15 μmol/L. In some embodiments, the enzymatically effective amount of saxiphilin specific for a toxin comprises about 20 μmol/L. In some embodiments, the enzymatically effective amount of saxiphilin specific for a toxin comprises about 30 μmol/L. In some embodiments, the enzymatically effective amount of saxiphilin specific for a toxin comprises about 40 μmol/L. In some embodiments, the enzymatically effective amount of saxiphilin specific for a toxin comprises about 50 μmol/L. In some embodiments, the enzymatically effective amount of saxiphilin specific for a toxin comprises about 60 μmol/L. In some embodiments, the enzymatically effective amount of saxiphilin specific for a toxin comprises about 70 μmol/L. In some embodiments, the enzymatically effective amount of saxiphilin specific for a toxin comprises about 80 μmol/L. In some embodiments, the enzymatically effective amount of saxiphilin specific for a toxin comprises about 90 μmol/L. In some embodiments, the enzymatically effective amount of saxiphilin specific for a toxin comprises about 100 μmol/L.

To facilitate the detection of a toxin disclosed herein, such as saxitoxin, within a sample, a detectable substance may be pre-applied to a surface, for example a plate, well, bead, nanodroplet, or other solid support comprising one or a plurality of reaction vessels. In some embodiments, a sample may be pre-mixed with a diluent or reagent before it is applied to a surface. The detectable substance may function as a detection probe that is detectable either visually or by an instrumental device. Any substance generally capable of producing a signal that is detectable visually or by an instrumental device may be used as detection probes. Suitable detectable substances may include, for instance, luminescent compounds (e.g., fluorescent, phosphorescent, etc.); radioactive compounds; visual compounds (e.g., colored dye or metallic substance, such as gold); liposomes or other vesicles containing signal-producing substances; enzymes and/or substrates, and so forth. Other suitable detectable substances may be described in U.S. Pat. No. 5,670,381 to Jou, et al. and U.S. Pat. No. 5,252,459 to Tarcha, et al., which are incorporated herein in their entirety by reference thereto for all purposes. If the detectable substance is colored, the ideal electromagnetic radiation is light of a complementary wavelength. For instance, blue detection probes strongly absorb red light.

In some embodiments, the detectable substance may be a luminescent compound that produces an optically detectable signal that corresponds to the level or quantity of a toxin in the sample. For example, suitable fluorescent molecules may include, but are not limited to, fluorescein, europium chelates, phycobiliprotein, phycoerythrin, phycocyanin, allophycocyanin, o-phthaldehyde, fluorescamine, rhodamine, and their derivatives and analogs. Other suitable fluorescent compounds are semiconductor nanocrystals commonly referred to as “quantum dots.” For example, such nanocrystals may contain a core of the formula CdX, wherein X is Se, Te, S, and so forth. The nanocrystals may also be passivated with an overlying shell of the formula YZ, wherein Y is Cd or Zn, and Z is S or Se. Other examples of suitable semiconductor nanocrystals may also be described in U.S. Pat. No. 6,261,779 to Barbera-Guillem, et al. and U.S. Pat. No. 6,585,939 to Dapprich, which are incorporated herein in their entirety by reference thereto for all purposes.

Further, suitable phosphorescent compounds may include metal complexes of one or more metals, such as ruthenium, osmium, rhenium, iridium, rhodium, platinum, indium, palladium, molybdenum, technetium, copper, iron, chromium, tungsten, zinc, and so forth. Especially preferred are ruthenium, rhenium, osmium, platinum, and palladium. The metal complex may contain one or more ligands that facilitate the solubility of the complex in an aqueous or non-aqueous environment. For example, some suitable examples of ligands include, but are not limited to, pyridine; pyrazine; isonicotinamide; imidazole; bipyridine; terpyridine; phenanthroline; dipyridophenazine; porphyrin; porphine; and derivatives thereof. Such ligands may be, for instance, substituted with alkyl, substituted alkyl, aryl, substituted aryl, aralkyl, substituted aralkyl, carboxylate, carboxaldehyde, carboxamide, cyano, amino, hydroxy, imino, hydroxycarbonyl, aminocarbonyl, amidine, guanidinium, ureide, sulfur-containing groups, phosphorus containing groups, and the carboxylate ester of N-hydroxy-succinimide.

Porphyrins and porphine metal complexes possess pyrrole groups coupled together with methylene bridges to form cyclic structures with metal chelating inner cavities. Many of these molecules exhibit strong phosphorescence properties at room temperature in suitable solvents (e.g., water) and an oxygen-free environment. Some suitable porphyrin complexes that are capable of exhibiting phosphorescent properties include, but are not limited to, platinum (II) coproporphyrin-I and III, palladium (II) coproporphyrin, ruthenium coproporphyrin, zinc(II)-coproporphyrin-I, derivatives thereof, and so forth. Similarly, some suitable porphine complexes that are capable of exhibiting phosphorescent properties include, but not limited to, platinum(II) tetra-meso-fluorophenylporphine and palladium(II) tetra-meso-fluorophenylporphine. Still other suitable porphyrin and/or porphine complexes are described in U.S. Pat. No. 4,614,723 to Schmidt, et al.; U.S. Pat. No. 5,464,741 to Hendrix; U.S. Pat. No. 5,518,883 to Soini; U.S. Pat. No. 5,922,537 to Ewart et al.; U.S. Pat. No. 6,004,530 to Sagner, et al.; and U.S. Pat. No. 6,582,930 to Ponomarev, et al., which are incorporated herein in their entirety by reference thereto for all purposes.

Bipyridine metal complexes may also be utilized as phosphorescent compounds. Some examples of suitable bipyridine complexes include, but are not limited to, bis[(4,4′-carbomethoxy)-2,2′-bipyridine]2-[3-(4-methyl-2,2′-bipyridine-4-yl)propyl]-1,3-dioxolane ruthenium (II); bis(2,2′bipyridine)[4-(butan-1-al)-4′-methyl-2,2′-bi-pyridine]ruthenium (II); bis(2,2′-bipyridine)[4-(4′-methyl-2,2′-bipyridine-4′-yl)-butyric acid] ruthenium (II); tris(2,2′bipyridine)ruthenium (II); (2,2′-bipyridine) [bis-bis(1,2-diphenylphosphino)ethylene]2-[3-(4-methyl-2,2′-bipyridine-4′-yl)propyl]-1,3-dioxolane osmium (II); bis(2,2′-bipyridine)[4-(4′-methyl-2,2′-bipyridine)-butylamine]ruthenium (II); bis(2,2′-bipyridine)[1-bromo-4(4′-methyl-2,2′-bipyridine-4-yl)butan-e]ruthenium (II); bis(2,2′-bipyridine)maleimidohexanoic acid, 4-methyl-2,2′-bipyridine-4′-butylamide ruthenium (II), and so forth. Still other suitable metal complexes that may exhibit phosphorescent properties may be described in U.S. Pat. No. 6,613,583 to Richter, et al.; U.S. Pat. No. 6,468,741 to Massey, et al.; U.S. Pat. No. 6,444,423 to Meade, et al.; U.S. Pat. No. 6,362,011 to Massey, et al.; U.S. Pat. No. 5,731,147 to Bard, et al.; and U.S. Pat. No. 5,591,581 to Massey, et al., which are incorporated herein by reference in their entireties.

In some cases, luminescent compounds may have a relatively long emission lifetime and/or may have a relatively large “Stokes shift.” The term “Stokes shift” is generally defined as the displacement of spectral lines or bands of luminescent radiation to a longer emission wavelength than the excitation lines or bands. A relatively large Stokes shift allows the excitation wavelength of a luminescent compound to remain far apart from its emission wavelengths and is desirable because a large difference between excitation and emission wavelengths makes it easier to eliminate the reflected excitation radiation from the emitted signal. Further, a large Stokes shift also minimizes interference from luminescent molecules in the sample and/or light scattering due to proteins or colloids, which are present with some body fluids (e.g., blood). In addition, a large Stokes shift also minimizes the requirement for expensive, high-precision filters to eliminate background interference. For example, in some embodiments, the luminescent compounds have a Stokes shift of greater than about 50 nanometers, in some embodiments greater than about 100 nanometers, and in some embodiments, from about 100 to about 350 nanometers.

For example, exemplary fluorescent compounds having a large Stokes shift include lanthanide chelates of samarium (Sm (III)), dysprosium (Dy (III)), europium (Eu (III)), and terbium (Tb (I)). Such chelates may exhibit strongly red-shifted, narrow-band, long-lived emission after excitation of the chelate at substantially shorter wavelengths. Typically, the chelate possesses a strong ultraviolet excitation band due to a chromophore located close to the lanthanide in the molecule. Subsequent to excitation by the chromophore, the excitation energy may be transferred from the excited chromophore to the lanthanide. This is followed by a fluorescence emission characteristic of the lanthanide. Europium chelates, for instance, have Stokes shifts of about 250 to about 350 nanometers, as compared to only about 28 nanometers for fluorescein. Also, the fluorescence of europium chelates is long-lived, with lifetimes of about 100 to about 1000 microseconds, as compared to about 1 to about 100 nanoseconds for other fluorescent labels. In addition, these chelates have narrow emission spectra, typically having bandwidths less than about 10 nanometers at about 50% emission. One suitable europium chelate is N-(p-isothiocyanatobenzyl)-diethylene triamine tetraacetic acid-Eu³.

Detectable substances (such as those capable of associating with or reacting to the presence of the reaction products cleaved by the proteases described herein), such as described above, may be used alone or in conjunction with a particle (sometimes referred to as “beads” or “microbeads”). For instance, naturally occurring particles, such as nuclei, mycoplasma, plasmids, plastids, mammalian cells (e.g., erythrocyte ghosts), unicellular microorganisms (e.g., bacteria), polysaccharides (e.g., agarose), etc., may be used. Further, synthetic particles may also be utilized. For example, in one embodiment, latex microparticles that are labeled with a fluorescent or colored dye are utilized. Although any synthetic particle may be used in the present invention, the particles are typically formed from polystyrene, butadiene styrenes, styreneacrylic-vinyl terpolymer, polymethylmethacrylate, polyethylmethacrylate, styrene-maleic anhydride copolymer, polyvinyl acetate, polyvinylpyridine, polydivinylbenzene, polybutyleneterephthalate, acrylonitrile, vinylchloride-acrylates, and so forth, or an aldehyde, carboxyl, amino, hydroxyl, or hydrazide derivative thereof. Other suitable particles may be described in U.S. Pat. No. 5,670,381 to Jou, et al.; U.S. Pat. No. 5,252,459 to Tarcha, et al.; and U.S. Patent Publication No. 2003/0139886 to Bodzin, et al., which are incorporated herein in their entirety by reference thereto for all purposes. Commercially available examples of suitable fluorescent particles include fluorescent carboxylated microspheres sold by Molecular Probes, Inc. under the trade names “FluoSphere” (Red 580/605) and “TransfluoSphere” (543/620), as well as “Texas Red” and 5- and 6-carboxytetramethylrhodamine, which are also sold by Molecular Probes, Inc. In addition, commercially available examples of suitable colored, latex microparticles include carboxylated latex beads sold by Bang's Laboratory, Inc. Metallic particles (e.g., gold particles) may also be utilized in the present invention.

When utilized, the shape of the particles may generally vary. In one particular embodiment, for instance, the particles are spherical in shape. However, it should be understood that other shapes are also contemplated by the present invention, such as plates, rods, discs, bars, tubes, irregular shapes, etc. In addition, the size of the particles may also vary. For instance, the average size (e.g., diameter) of the particles may range from about 0.1 nanometers to about 100 microns, in some embodiments, from about 1 nanometer to about 10 microns, and in some embodiments, from about 10 to about 100 nanometers.

In some embodiments, the system disclosed herein comprises a chip, slide or other silica surface comprising one or a plurality of addressable locations or reaction vessels within which one or a plurality of saxiphilins with an affinity for the toxins disclosed herein are immobilized or contained. Upon contacting a sample comprising a toxin with an affinity for the saxiphilins disclosed herein, a reaction ensues whose reaction products are detectable by any means known in the art or disclosed herein. For instance, the reaction products may be detectable by fluorescence, optical imaging, field microscopy, mass spectrometry, or the like.

### Methods

In some embodiments, the disclosure relates to a method of detecting the presence, absence or quantity of toxin; wherein the amount of toxin in a sample is determined by calculating the amount of intensity or presence of color caused by a colorimetric substance that forms in proportion to the amount of cleaved saxiphilin in a reaction vessel. Colorimetric assays may be used in vitro when a probe comprises a saxiphilin specific for the toxin is bound, noncovalently or covalently, to a colorimetric substrate. In some embodiments, the disclosure relates to a method of detecting the presence, absence or quantity of a toxin wherein the amount of toxin in a sample is determined by calculating the amount of intensity or presence of color caused by a colorimetric substance that forms in proportion to the amount of cleaved saxiphilin in a reaction vessel. Colorimetric assays may be used in vitro when a probe comprises a saxiphilin specific for the toxin is bound, noncovalently or covalently, to a colorimetric substrate.

In some embodiments, the disclosure relates to a method of detecting the presence, absence or quantity of a toxin and wherein the amount of toxin in a sample is determined by calculating the amount of intensity or presence of color caused by a colorimetric substance that forms in proportion to the amount of cleaved saxiphilin in a reaction vessel. Colorimetric assays may be used in vitro when a probe comprises a saxiphilin specific for the toxin is bound, noncovalently or covalently, to a colorimetric substrate. In some embodiments, the disclosure relates to a method of detecting the presence, absence or quantity of a toxin wherein the amount of toxin in a sample is determined by calculating the amount of intensity or presence of color caused by a colorimetric substance that forms in proportion to the amount of cleaved saxiphilin in a reaction vessel. Colorimetric assays may be used in vitro when a probe comprises a saxiphilin specific for a toxin is bound, noncovalently or covalently, to a colorimetric substrate.

In some embodiments, any of the methods disclosed herein comprise a step of detecting the presence or quantity of a toxin with a sensitivity of no less than about 10 nM of toxin concentration in a sample. In some embodiments, any of the methods disclosed herein comprise a step of detecting the presence or quantity of a toxin with a sensitivity of no less than about 10, 11, 12, 13, 14, 15, 16, 17, 18, 19, 20, 21, 22, 23, 24, 25, 30, 35, 40, 45, 50, 55, 60, 65, 70, 75, 80, 85, 90, 91, 92, 93, 94, or 95 nM of toxin concentration in a sample. In some embodiments, any of the methods disclosed herein comprise a step of detecting the presence or quantity of a toxin with a sensitivity of no less than about 10 nM of toxin concentration in a sample. In some embodiments, any of the methods disclosed herein comprise a step of detecting the presence or quantity of a toxin with a sensitivity of no less than about 10, 11, 12, 13, 14, 15, 16, 17, 18, 19, 20, 21, 22, 23, 24, 25, 30, 35, 40, 45, 50, 55, 60, 65, 70, 75, 80, 85, 90, 91, 92, 93, 94, or 95 nM of toxin concentration in a sample.

In some embodiments, the disclosure relates to a method of detecting the presence, absence or quantity of a toxin, including but not limited to saxitoxin, in a sample where the amount of saxiphilin in a sample is determined by calculating the amount of fluorescence of a cleaved substrate in a reaction vessel and is calculated by the expression: (F_{final}−Fᵢₙᵢₜᵢₐₗ)/Fᵢₙᵢₜᵢₐₗ, wherein F stands for relative fluorescence units (RFU) and is a standard plate reader unit, where the amount of fluorescent signal detected is linearly or substantially linearly related to the amount of saxiphilin in a sample. In some embodiments, a threshold amount or biologically significant amount of toxin, for the amount in a sample which may indicate paralytic shellfish poisoning, is about a 1.19, 1.20, 1.21, 1.22, 1.23, 1.24, 1.25, 1.26, 1.27, 1.28, 1.29 fold-change in fluorescence. In some embodiments, the sensitivity can be equal to detection of toxin at a level of about 0.125 nM within the sample. In some embodiments, the sensitivity can be equal to detection of toxin at a level of about 0.120 nM within the sample. In some embodiments, the sensitivity can be equal to detection of toxin at a level of about 0.110 nM within the sample. In some embodiments, the sensitivity can be equal to detection of toxin at a level of about 0.140 nM within the sample. In some embodiments, the sensitivity can be equal to detection of toxin at a level of about 0.130 nM within the sample. In some embodiments, the sensitivity can be equal to detection of toxin at a level of about 0.150 nM within the sample. In some embodiments, the sensitivity can be equal to detection of toxin at a level of about 0.225 nM within the sample. In some embodiments, the sensitivity can be equal to detection of toxin at a level of about 0.175 nM within the sample. In some embodiments, the sensitivity can be equal to detection of toxin at a level of about 0.125 μM within the sample. In some embodiments, the sensitivity can be equal to detection of toxin at a level of about 0.200 μM within the sample.

In some embodiments, the method of the disclosure further comprises detecting the presence, absence or quantity of toxin in the sample where the amount of toxin in the sample is determined by calculating the amount of fluorescence of a cleaved substrate in the reaction vessel and is calculated by the expression: (F_{final}−Fᵢₙᵢₜᵢₐₗ)/Fᵢₙᵢₜᵢₐₗ discussed above. A biologically significant amount of toxin (e.g. for the amount in a sample which may paralytic shellfish poisoning), is at least about a 1.19, 1.20, 1.21, 1.22, 1.23, 1.24, 1.25, 1.26. 1.27, 1.28, 1.29 fold-change in fluorescence. In some embodiments, the sensitity can be equal to detection of toxin at a level of about 0.125 μM within the sample. In some embodiments, the sensitivity can be equal to detection of toxin at a level of about 0.120 μM within the sample. In some embodiments, the sensitivity can be equal to detection of toxin at a level of about 0.110 μM within the sample. In some embodiments, the sensitivity can be equal to detection of toxin at a level of about 0.140 μM within the sample. In some embodiments, the sensitivity can be equal to detection of toxin at a level of about 0.130 μM within the sample. In some embodiments, the sensitivity can be equal to detection of toxin at a level of about 0.150 μM within the sample. In some embodiments, the sensitivity can be equal to detection of toxin at a level of about 0.225 μM within the sample. In some embodiments, the sensitivity can be equal to detection of toxin at a level of about 0.175 μM within the sample. In some embodiments, the sensitivity can be equal to detection of toxin at a level of about 0.125 μM within the sample. In some embodiments, the sensitivity can be equal to detection of toxin at a level of about 0.200 μM within the sample. In some embodiments, the sensitivity can be equal to detection of toxin at a level of about 0.090, 0.091, 0.92, 0.093, 0.094, 0.095 μM within the sample. In some embodiments, the sensitivity can be equal to detection of toxin at a level of from about 10, 11, 12, 13, 14, or 15 nM to about 100 nM within the sample.

In some embodiments, the disclosure relates to methods of diagnosing subjects comprising detecting the amount of toxin present in a sample from a patient. The present disclosure also relates to methods comprising detecting the amount of toxin present in a sample from a shellfish. In one aspect, the method of detecting the amount of toxin comprises: (a) contacting a plurality of probes specific for a toxin and/or functional fragment thereof with a sample; (b) quantifying the amount of a toxin and/or functional fragment thereof in the sample; (c) calculating one or more normalized scores based upon the presence, absence, or quantity of a toxin and/or functional fragment thereof; and (d) correlating the one or more scores to the presence, absence, or quantity of a toxin and/or functional fragment thereof, such that if the amount of a toxin and/or functional fragment thereof is greater than the quantity of a toxin and/or functional fragment thereof in a control sample, the correlating step comprises characterizing the sample as comprising a toxin.

The present disclosure relates to the detecting a toxin, such as saxitoxin, in a subject, the method comprising: (i) obtaining a sample from the subject; and; (ii) detecting whether a toxin, such as saxitoxin, is present at biologically significant levels within the sample by contacting the sample with a probe or saxiphilin specific for a toxin, such as saxitoxin, and detecting binding between a toxin, such as saxitoxin, and the probe or saxiphilin. In some embodiments, the biologically significant levels of a toxin, such as saxitoxin, and/or functional fragments thereof within a sample are at or greater than about a 1 fold change in quantity as compared to the amount of a toxin, such as saxitoxin, or functional fragments thereof in a control sample (for instance, a sample known to not contain a toxin). In some embodiments, the biologically significant levels of a toxin, such as saxitoxin, and/or functional fragments thereof within a sample are at or greater than about a 1.1 fold change as compared to the amount of toxin, such as saxitoxin, or functional fragments thereof in a control sample (for instance, a sample known to not contain a toxin). In some embodiments, the biologically significant levels of a toxin, such as saxitoxin, and/or functional fragments thereof within a sample are at or greater than about a 1.2 fold change as compared to the amount of a toxin, such as saxitoxin, or functional fragments thereof in a control sample (for instance, a sample known to not contain a toxin). In some embodiments, the biologically significant levels of a toxin, such as saxitoxin, and/or functional fragments thereof within a sample are at or greater than about a 1.3 fold change as compared to the amount of a toxin, such as saxitoxin, or functional fragments thereof in a control sample (for instance, a sample known to not contain a toxin). In some embodiments, the biologically significant levels of a toxin, such as saxitoxin, and/or functional fragments thereof within a sample are at or greater than about a 1.4 fold change as compared to the amount of a toxin, such as saxitoxin, or functional fragments thereof in a control sample (for instance, a sample known to not contain a toxin). In some embodiments, the biologically significant levels of a toxin, such as saxitoxin, and/or functional fragments thereof within a sample are at or greater than about a 1.5 fold change as compared to the amount of a toxin, such as saxitoxin, or functional fragments thereof in a control sample (for instance, a sample known to not contain a toxin).

In some embodiments, any tissue or body fluid sample may be used to detect the absence or presence of a toxin. Examples of samples can include saliva, blood or urine. One skilled in the art would readily recognize other types of samples of methods of obtaining them. In some embodiments of the methods disclosed herein, any of the methods disclosed herein comprise a step of obtaining a sample from a subject such as a human patient.

Various formats may be used to test for the presence, absence or quantity of a toxin or functional fragment thereof using the assay devices or system of the present disclosure. For instance, a “sandwich” format typically involves mixing the test sample with probes conjugated with a specific binding member (e.g., antibody) for the analyte to form complexes between the analyte and the conjugated probes. These complexes are then allowed to contact a receptive material (e.g., antibodies) immobilized within the detection zone. Binding occurs between the analyte/probe conjugate complexes and the immobilized receptive material, thereby localizing “sandwich” complexes that are detectable to indicate the presence of the analyte. This technique may be used to obtain quantitative or semi-quantitative results. Some examples of such sandwich-type assays are described by U.S. Pat. No. 4,168,146 to Grubb, et al. and U.S. Pat. No. 4,366,241 to Tom, et al., which are incorporated herein in their entirety by reference thereto for all purposes. In a competitive assay, the labeled probe is generally conjugated with a molecule that is identical to, or an analog of, the analyte. Thus, the labeled probe competes with the analyte of interest for the available receptive material. Competitive assays are typically used for detection of analytes such as haptens, each hapten being monovalent and capable of binding only one antibody molecule. Examples of competitive immunoassay devices are described in U.S. Pat. No. 4,235,601 to Deutsch, et al., U.S. Pat. No. 4,442,204 to Liotta, and U.S. Pat. No. 5,208,535 to Buechler, et al., which are incorporated herein in their entirety by reference thereto for all purposes. Various other device configurations and/or assay formats are also described in U.S. Pat. No. 5,395,754 to Lambotte, et al.; U.S. Pat. No. 5,670,381 to Jou, et al.; and U.S. Pat. No. 6,194,220 to Malick, et al., which are incorporated herein in their entirety by reference thereto for all purposes.

Although various assay device configuration have been described herein, it should be understood that any known assay device may be utilized that is capable of incorporating an antibody in accordance with the present invention. For example, electrochemical affinity assay devices may also be utilized, which detect an electrochemical reaction between a lysosomal protease (or complex thereof) and a capture ligand on an electrode strip. For example, various electrochemical assays and assay devices are described in U.S. Pat. No. 5,508,171 to Walling, et al.; U.S. Pat. No. 5,534,132 to Vreeke, et al.; U.S. Pat. No. 6,241,863 to Monbouquette; U.S. Pat. No. 6,270,637 to Crismore, et al.; U.S. Pat. No. 6,281,006 to Heller, et al.; and U.S. Pat. No. 6,461,496 to Feldman, et al., which are incorporated herein in their entirety by reference thereto for all purposes.

One skilled in the art will readily appreciate the wide range of methods and techniques used for detecting the presence and/or quantity of toxins, proteins, enzymes and/or cleavage products in a complex sample. Techniques for detecting proteins or cleavage products include, but are not limited to, microscopy, immunostaining, immunoprecipitation, immunoelectrophoresis, Western blot, BCA assays, spectrophotometry, enzymatic assays, microchip assays, and mass spectrometry. In some embodiments, purification of proteins are necessary before detection of quantification techniques are employed. Techniques for purifying proteins include, but are not limited to, chromatography methods, including ion exchange, size-exclusion, and affinity chromatography, gel electrophoresis, magnetic beads comprising any antibody, antibody-like protein or antibody fragment or variant, Bradford protein assays. In some embodiments, methods of measuring the presence, absence, or quantity of a toxin or functional fragments thereof comprise antibodies or antibody fragments specific to a toxin or functional fragments thereof.

Any and all journal articles, patent applications, issued patents, or other cited references disclosed herein (including GenBank Accession numbers or other genetic information identification tags dated as of the date of the application filing) are incorporated by reference in their respective entireties. Any of the systems, methods or devices disclosed herein comprising saxiphilins may be made or performed or used with any of the saxiphilins disclosed herein. In some embodiments, the systems, methods or devices disclosed herein are selectively free of any one or combination of toxins disclosed herein.

### EXAMPLES

### Example 1: Definition of a Saxitoxin (STX) Binding Code Enables Discovery and Characterization of the Anuran Saxiphilin Family

To characterize RcSxph:STX interactions in detail, we developed a suite of assays comprising thermofluor (TF) measurements of ligand-induced changes in RcSxph stability, fluorescence polarization (FP) binding to a fluorescein-labeled STX, and isothermal titration calorimetry (ITC). We paired these assays with a scanning mutagenesis strategy (Clackson and Wells, Wells 1991) to dissect the energetic contributions of RcSxph STX binding pocket residues. These studies show that the core RcSxph STX recognition code comprises two ‘hot spot’ triads. One engages the STX tricyclic bis-guanidinium core through a pair of carboxylate groups and a cation-p interaction (Infield et al.) in a manner that underscores the convergent STX recognition strategies shared by RcSxph and Navs (Infield et al., Thomas-Tran and Du Bois, Heinemann et al., Satin et al., Leffler et al., Sivilotti et al.). The second triad largely interacts with the C13 carbamate group of STX and is the site of interactions that can enhance STX binding affinity and the ability of RcSxph to act as a ‘toxin sponge’ that can reverse the effects of STX inhibition of Navs (Mahar et al., Abderemaine-Ali et al.).

Although Sxph-like STX binding activity has been reported in extracts from diverse organisms including arthropods (Llewellyn et al.), amphibians (Doyle et al., Llewellyn et al., Tanaka et al.), fish (Llewellyn et al.), and reptiles (Llewellyn et al.), the molecular origins of this activity have remained obscure. Definition of the RcSxph STX recognition code enabled identification of ten new Sxphs from diverse frogs and toads. This substantial enlargement of the Sxph family beyond RcSxph and the previously identified High Himalaya frog (Nanorana parkeri) Sxph (NpSxph) (Yen et al.) reveals a varied STX binding pocket surrounding a conserved core of ‘hot spot’ positions and dramatic differences in the number of thyroglobulin (Thy1) domains inserted into the modified transferrin fold upon which Sxph is built. Biochemical characterization of NpSxph, Oophaga sylvatica Sxph (OsSxph) (Caty et al.), Mantella aurantica Sxph (MaSxph), and Ranitomeya imitator Sxph (RiSxph), together with structural determination of NpSxph shows that the different Sxphs retain the capacity for high affinity STX binding and that binding site preorganization (Yen et al.) is a critical factor for STX binding. Together, these studies establish a STX molecular recognition code that provides a framework for understanding how diverse STX binding proteins engage the toxin and its congeners and uncover that Sxph family members are abundantly found in the most varied and widespread group of amphibians, the Anurans.

### 1. Results

### Establishment of a Suite of Assays to Probe RcSxph Toxin Binding Properties

To investigate the molecular details of the high-affinity RcSxph:STX interaction, we developed three assays to assess the effects of STX binding site mutations. A key criterion was to create assays that could be performed in parallel on many RcSxph mutants using minimal amounts of purified protein and toxin. To this end, we first tested whether we could detect STX binding using a thermofluor (TF) assay (Huynh and Partch, Niesen et al.) in which STX binding would manifest as concentration-dependent change in the apparent RcSxph melting temperature (Tm). Addition of STX, but not the related guanidinium toxin, tetrodotoxin (TTX), over a 0-20 μM range to samples containing 1.1 μM RcSxph caused concentration-dependent shifts in the RcSxph melting cure (FIG. 1A)(DTm=3.6° C.±0.2 versus 0.3° C.±0.4 for STX and TTX, respectively). These differential effects of STX and TTX are in line with the ability of RcSxph to bind STX (Mahar et al., Llewellyn et al., Llewellyn and Moczydlowski) but not TTX (Mahar et al., Abderemane-Ali et al.) and indicate that DTm is a consequence of the RcSxph:STX interaction.

To investigate the contributions of residues that comprise the STX binding site, we coupled the TF assay with alanine scanning (Clackson and Wells) as well as deeper mutagenesis studies targeting the eight residues that directly contact STX (Glu540, Phe561, Thr563, Tyr558, Pro727, Phe784, Asp785, Asp794) (Yen et al.) and four second shell sites that support these residues (Tyr795, Ile782, Gln787, and Lys789) (FIGS. 1A-B and FIG. S1A). Measurement of the STX-induced DTm changes for these twelve, purified RcSxph alanine mutants revealed DTm changes spread over a ˜4° C. range that included DTm increases relative to wild-type (e.g. I782A and D785N) as well as those that caused complete loss of the thermal shift (e.g. E540A and D794A). All mutations had minimal effects on protein stability (FIG. S1B) and there was no evident correlation between Tm and DTm (FIG. S1C). Hence, the varied DTms indicate that each of the twelve positions contribute differently to STX binding.

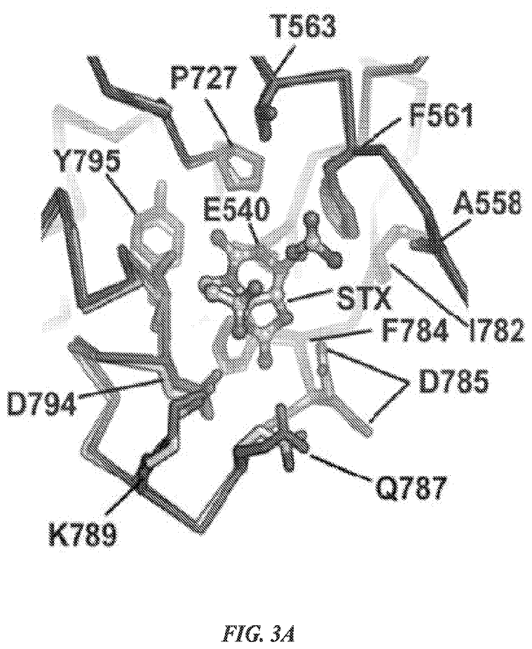

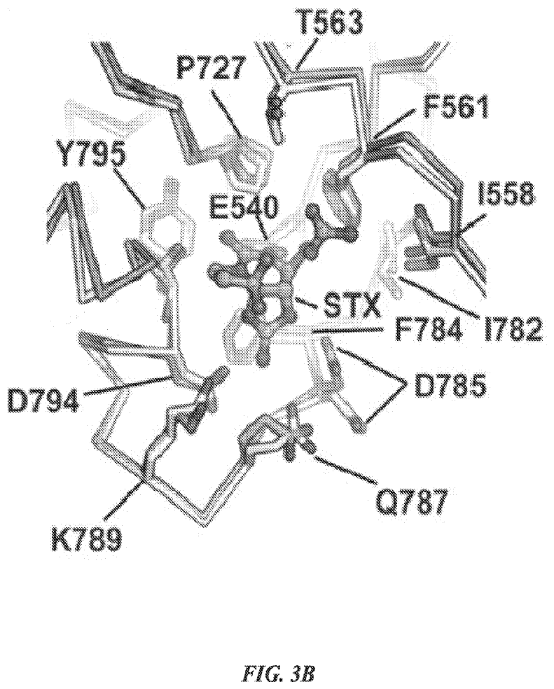

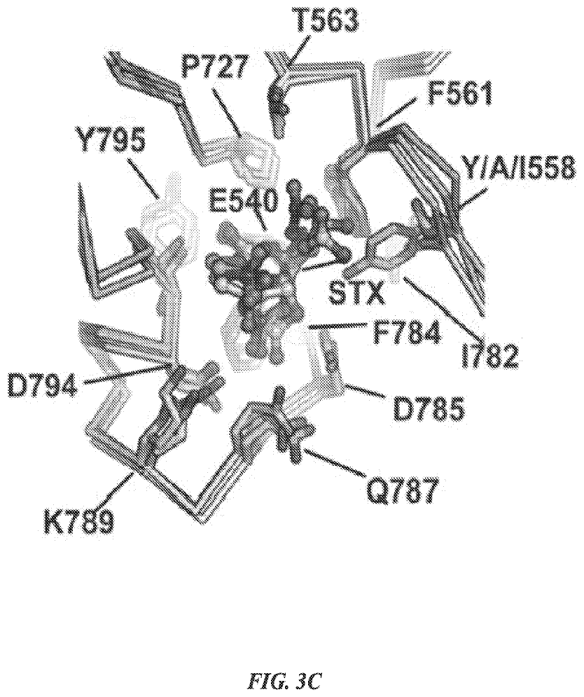

Because DTm interpretation can be complex, especially in the case of a multidomain protein such as RcSxph, and may not necessarily indicate changes in ligand affinity (Huynh and Partch, Niesen et al.), we developed a second assay to measure the effects of mutations on RcSxph binding affinity. We synthesized a fluorescein-labeled STX derivative (F-STX) by functionalization of the pendant carbamate group with a 6-carbon linker and fluorescein (Andresen and DuBois, Ondrus et al.) (FIGS. 1C and FIG. S2) and established a fluorescence polarization (FP) assay (Huang and Aulabaugh, Zhang et al.) to measure toxin binding. FP measurements revealed a high-affinity interaction between F-STX and RcSxph (Kd=7.4 nM±2.6) that closely agrees with prior radioligand assay measurements of RcSxph affinity for STX (˜1 nM) (Llewellyn and Moczydlowski). The similarity between the F-STX and STX Kd values is consistent with the expectation from the RcSxph:STX structure that STX carbamate derivatization should have a minimal effect on binding, as this element residues on the solvent exposed side of the STX binding pocket (Yen et al.). To investigate the F-STX interaction further, we soaked RcSxph crystals with F-STX and determined the structure of the RcSxph:F-STX complex at 2.65A resolution by X-ray crystallography (FIG. 3A, Table S1). Inspection of the STX binding pocket revealed clear electron density for the F-STX bis-guanidinium core as well as weaker density that we could assign to the fluorescein heterocycle (FIG. S3A), although the high B-factors of the linker and fluorescein indicate that these moieties are highly mobile (FIG. S3B). Structural comparison with the RcSxph:STX complex (Yen et al.) showed no changes in the core STX binding pose or STX binding pocket residues (RMSD_{Ca}=0.279A) (FIG. S3C). Together, these data demonstrate that both F-STX and STX bind to Sxph in the same manner and indicate that there are no substantial interactions with the fluorescein label.

FP measurement of the RcSxph alanine scan mutants uncovered binding affinity changes spanning a ˜13,000 fold range that correspond to free energy perturbations (DDG) of up to ˜5.60 kcal mol⁻¹ (FIG. 1D and FIG. S4, Table 1). The effects were diverse, encompassing enhanced affinity changes (Y558A Kd=1.2 nM±0.3) and large disruptions (E540A Kd=15.3 μM±4.1). As indicated by the TF data, each STX binding pocket residue contributes differently to STX recognition energetics. Comparison of the TF DTm and FP DDG values shows a strong correlation between the two measurements (FIG. 1E) and indicates that the changes in unfolding free energies caused by protein mutation and changes in STX binding affinity do not incur large heat capacity or entropy changes relative to the wild-type protein (Becktel and Schellman, Arrigoni and Minor). Hence, DTm values provide an accurate estimate of the STX binding affinity differences.

TABLE 1
RcSxph STX binding pocket mutant binding parameters

| Class | Construct | Kd (nM) | □□G (kcal mol⁻¹) | n |
|---|---|---|---|---|
| Enhanced binding | I782A/Y558A | 1.2 ± 0.2 | −1.07 | 6 |
| □□G < 1 kcal mol⁻¹ | Y558I | 1.2 ± 0.2 | −1.07 | 6 |
| | Y558A | 1.4 ± 0.3 | −1.00 | 6 |
| Mild enhancement | I782A | 3.0 ± 0.8 | −0.53 | 4 |
| 1 ≤ □□G ≤ 0 kcal mol⁻¹ | D785N | 4.4 ± 0.6 | −0.30 | 4 |
| | K789A | 5.1 ± 1.7 | −0.22 | 4 |
| | T563A | 5.3 ± 0.5 | −0.20 | 6 |
| | Y558F | 6.3 ± 2.3 | −0.10 | 4 |
| | Q787E | 6.4 ± 1.6 | −0.09 | 4 |
| | RcSxph | 7.4 ± 2.6 | 0 | 10 |
| Mild disruption | Y795A | 8.4 ± 2.1 | 0.08 | 4 |
| 0 ≥ □□G ≥ 1 kcal mol⁻¹ | Q787A | 11.3 ± 1.1 | 0.25 | 4 |
| | F784Y | 13.8 ± 1.0 | 0.37 | 4 |
| | I782F | 16.1 ± 4.1 | 0.46 | 4 |
| | F561A | 16.8 ± 6.0 | 0.48 | 4 |
| | D785A | 19.5 ± 2.5 | 0.57 | 4 |
| Disruption | F784L | 48.0 ± 6.8 | 1.11 | 4 |
| 1 ≥ □□G ≥ 2 kcal mol⁻¹ | P727A | 56.9 ± 12.1 | 1.21 | 4 |
| | E540D | 99.9 ± 25.1 | 1.54 | 4 |
| Strong disruption | F784A | 725.1 ± 108.7 | 2.71 | 4 |
| 2 ≥ □□G ≥ 3 kcal mol⁻¹ | D794E | 1074.1 ± 69.3 | 2.94 | 4 |
| Very strong disruption | F784C | 1510.5 ± 3 46.1 | 3.15 | 4 |
| □□G ≥ 3 | D794N | 3228 ± 397 | 3.60 | 4 |
| | F784S | 3240 ± 508 | 3.60 | 4 |
| | E540Q | 10640 ± 1325 | 4.30 | 4 |
| | D794A | 13172 ± 6871 | 4.43 | 4 |
| | E540A | 15294 ± 4134 | 4.52 | 10 |

Kd, dissociation constant;
n, number of observations
□□G = RT ln (Kd_{Sxph mutant}/Kd_{Sxph}); T = 298K
Errors for measurements are S.D.

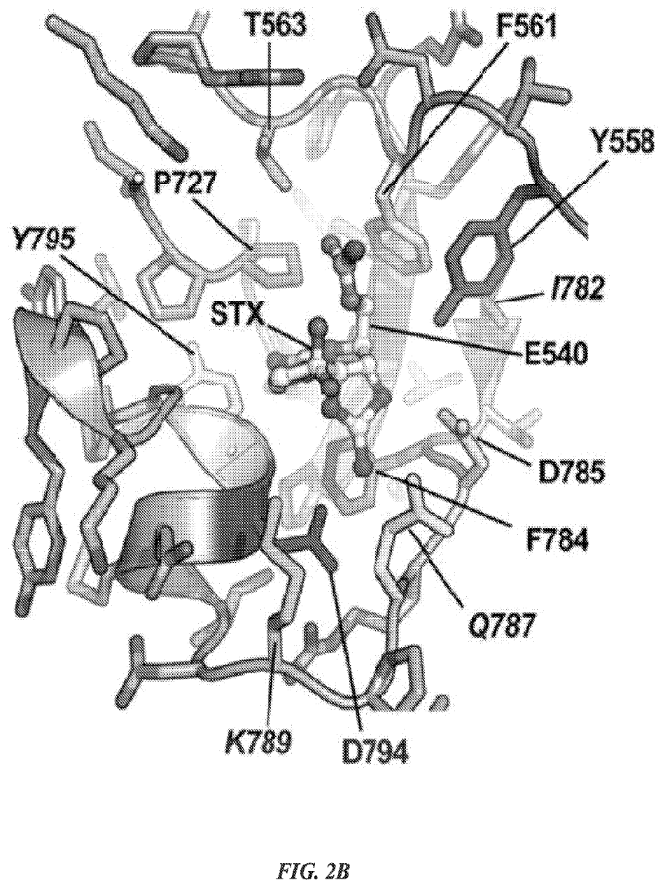

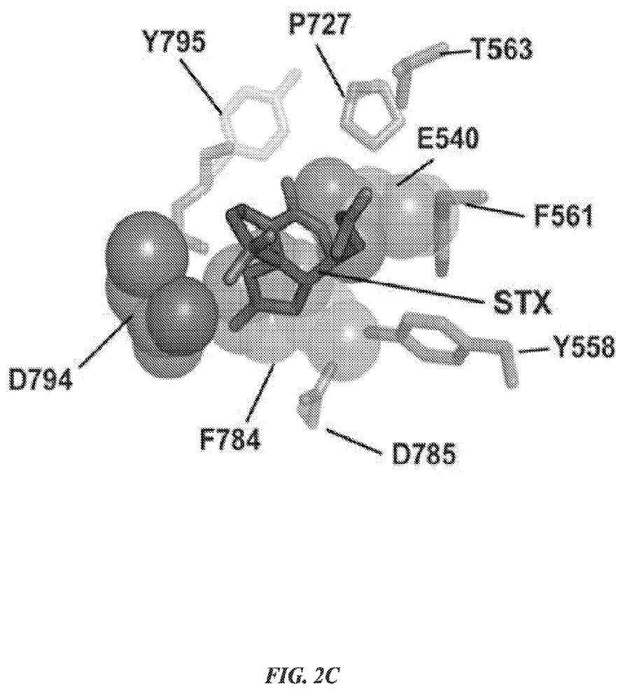

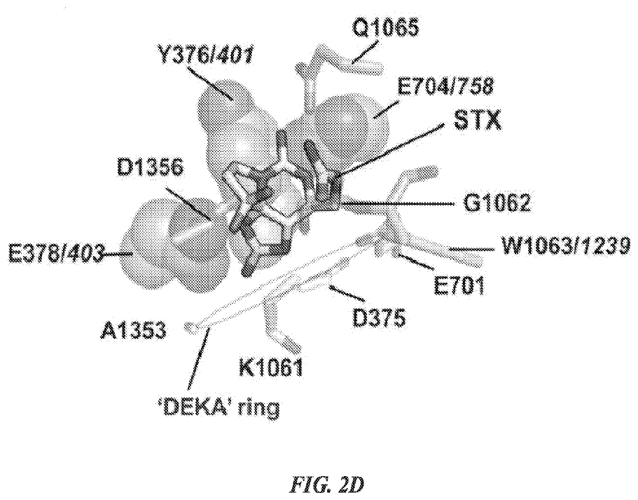

To investigate the STX affinity changes further, we used isothermal titration calorimetry (ITC) (FIGS. 1F, and Table S2), a label-free method that reports directly on ligand association energetics (Velazquez-Campoy et al.), to examine the interaction of STX with RcSxph and six mutants having varied effects on binding (E540D, Y558I, Y558A, F561A, P727A, and D794E) (FIGS. 1F, 2A, S5A-C, and Table S2). Experiments with RcSxph confirm the 1:1 stoichiometry and high affinity of the RcSxph:STX interaction (Kd ˜nM) reported previously (Mahar et al., Yen et al., Llewellyn and Moczydlowski) and reveal a large, favorable binding enthalpy (DH −16.1±0.2 kcal mol⁻¹) in line with previous radioligand binding studies (Llewellyn and Moczydlowski). In almost all mutants, binding affinity loss correlated with a reduction in enthalpy, consistent with a loss of interactions (Table S2). The one exception to this trend is E540D for which STX association yielded a binding enthalpy (DH −16.3±1.7 kcal mol⁻¹) very similar to wild type RcSxph that was offset by a ˜two-fold unfavorable change in binding entropy. The ITC measurements were unable to measure the affinity enhancement for Y558A and Y558I accurately due to the fact that these mutants, as well as RcSxph have Kds at the detection limit of direct titration methods (˜1 nM) (Velazquez-Campoy et al.). Nevertheless, DG_{ITC} from mutants having STX Kds within the ITC dynamic range (Kds ˜30-300 nM) showed an excellent agreement with DG_{FP} measurements made with F-STX (FIG. 1G). These data further validate the TF and FP assay trends and support the conclusion that RcSpxh:F-STX binding interactions are very similar to the RcSpxh:STX interactions. Together, these three assays (FIGS. 1E and G) provide a robust and versatile suite of options for characterizing STX:Sxph interactions.

### Sxph STX Binding Code is Focused on Two Sets of ‘Hot Spot’ Residues

To understand the structural code underlying STX binding, we classified the effects of the alanine mutations into six groups based on DDG values (FIG. 2A and Table 1) and mapped these onto the RcSxph structure (FIG. 2B). This analysis identified a binding ‘hot spot’ comprising three residues that directly contact the STX bis-guanidinium core (Glu540, Phe784, and Asp794) (Yen et al.) and an additional site near the carbamate (Pro727) where alanine mutations caused substantial STX binding losses (DDG≥1 kcal mol⁻¹). Conversely, we also identified a site (Tyr558) where alanine caused a notable enhancement of STX binding (DDG≤−1 kcal mol⁻¹) (FIG. 2B, Table 1).

To examine the physicochemical nature of key residues critical for STX binding further, we made mutations at select positions guided by the alanine scan. Mutations at Glu540 and Asp794 (Yen et al.), residues involved in charge pair interactions with the STX guanidinium rings, that neutralized the sidechain while preserving shape and volume (FIG. 2A, Table 1) disrupted binding strongly, similar to their alanine counterparts (DDG=4.30 and 3.60 kcal mol¹, for E540Q and D794N, respectively) (FIGS. 2A, S4, Table 1). Altering sidechain length while preserving the negative charge at these sites also greatly diminished STX affinity, but was notably less problematic at Glu540 (DDG=1.54 and 2.94 kcal mol⁻¹, for E540D and D794E, respectively). To probe contacts with Phe784, which makes a cation-p interaction (Infield et al.) with the STX five-membered guanidinium ring (Yen et al.), we tested changes that preserved this interface (F784Y), maintained sidechain volume and hydrophobicity (F784L), and that mimicked substitutions (F784C and F784S) found in the analogous residue in STX-resistant Na_{V}s (Na_{V}1.5, Na_{V}18 and Na_{V}19) (Yen et al., Infield et al., Heinemann et al., Satin et al., Leffler et al., Sivilotti et al.) (FIG. 2A, Table 1). Preserving the cation-p interaction with F784Y caused a modest binding reduction (DDG=0.37 kcal mol⁻¹), whereas F784L was disruptive (DDG=1.11 kcal mol⁻¹) and F784C and F784S were even more destabilizing than F784A (DDG=3.15, 3.60, and 2.71 kcal mol⁻¹, respectively).

We also examined two other positions that form part of the Sxph binding pocket near the five-membered STX guanidinium ring. Asp785 undergoes the most dramatic conformational change of any residue associated with STX binding moving from an external facing conformation to one that engages this STX element (Yen et al.). Surprisingly, D785A and D785N mutations caused only relatively modest binding changes (FIG. 2A, Table 1) (DDG=0.57 and −0.30 kcal mol⁻¹, for D785A and D785N, respectively). Because of the proximity of the second shell residue Gln787 to Asp785 and Asp794 (FIG. 2B), two residues that coordinate the five-membered STX guanidinium ring (Yen et al.), we also asked whether adding additional negative charge to this part of the STX binding pocket would enhance toxin binding affinity. However, Q787E had essentially no effect on binding (DDG=−0.09 kcal mol¹).

Two residues, Tyr558 and Ile782, stood out as sites where alanine substitutions enhanced STX affinity (FIGS. 2A-B, Table 1). Tyr558 interacts with both the STX five-membered guanidinium ring and carbamate and moves away from the STX binding pocket upon toxin binding (Yen et al.), whereas Ile782 is a second shell site that buttresses Tyr558. Hence, we hypothesized that affinity enhancements observed in the Tyr558 and Ile782 mutants resulted from the reduction of Tyr558-STX clashes. In accord with this idea, Y558F had little effect on STX binding (DDG=−0.10 kcal mol¹), whereas shortening the sidechain but preserving its hydrophobic character, Y558I, enhanced binding as much as Y558A (DDG=−1.07 and −1.00 kcal mol⁻¹, respectively). Increasing the sidechain volume at the buttressing position, I782F, reduced STX binding affinity (DDG=0.46 kcal mol⁻¹). Combining the two affinity enhancing mutants, Y558A/I782A, yielded only a marginal increase in affinity in comparison to Y558A (DDG=−1.07 and −1.00 kcal mol⁻¹, respectively) but was better than I782A alone (DDG=−0.53 kcal mol⁻¹). This non-additivity in binding energetics (Wells, 1990) is in line with the physical interaction of the two sites and the direct contacts of Tyr558 with the toxin. Together, these data support the idea that the Tyr558 clash with STX is key factor affecting STX affinity and suggest that it should be possible to engineer Sxph variants with enhanced binding properties by altering this site.

Collectively, this energetic map of the RcSxph STX binding pocket highlights the importance of two amino acid triads. One (Glu540, Phe784, and Asp794) engages the STX bis-guanidinium core of the toxin. The second (Tyr558, Phe561, and Pro727), forms the surface surrounding the carbamate unit (FIG. 2B). The central role of the Glu540/Phe784/Asp794 triad in the energetics of binding the bis-guanidinium STX core underscores the toxin receptor site similarities between RcSxph and Na_{V}s (Yen et al.) (FIGS. 2C-D). In both, STX binding relies on two acidic residues that coordinate the five and six-membered STX rings (Sxph Asp794 and Glu540, and rat Na_{V}1.4 Glu403 and Glu758 (Thomas-Tran and DuBois)), and a cation-p interaction (Sxph Phe784 and ratNa_{V}1.4 Tyr401 (Thomas-Tran and DuBois) and its equivalents in other Na_{V}s (Heinemann et al., Satin et al., Leffler et al., Sivilotti et al.)). Hence, both the basic architecture and binding energetics appear to be conserved even though the overall protein structures presenting these elements are dramatically different.

### Structures of Enhanced Affinity RcSxph Mutants

To investigate the structural underpinnings of the affinity enhancement caused by mutations at the Tyr558 site, we determined crystal structures of RcSxph-Y558A and RcSxph-Y558I a lone (2.60 Å and 2.70 Å resolution, respectively) and as co-crystallized STX complexes (2.60 Å and 2.15A, respectively) (FIG. S6A-D, Table S1). Comparison of the apo- and STX-bound structures reveals little movement in the STX binding pocket upon ligand binding (RMSD_{Ca}=0.209 Å and 0.308 Å comparing apo- and STX-bound RcSxph-Y558 Å and RcSxph-Y558I, respectively) (FIGS. 3A-B, Supplementary movies, M1 and M2). In both, the largest conformational change is the rotation of Asp785 into the binding pocket to interact with the five-membered guanidinium ring of STX, as seen for RcSxph (FIG. 3C) (Yen et al.). By contrast, unlike in RcSxph, there is minimal movement of residue 558 and its supporting loop, indicating that both Y558 Å and Y558I eliminate the clash incurred by the Tyr558 sidechain. Comparison with the RcSxph:STX complex also shows that the STX carbamate in both structures has moved into a pocket formed by the mutation at Tyr558 (RMSD_{Ca}=0.279 Å and 0.327 Å comparing RcSxph:STX with RcSxph-Y558A:STX and RcSxph-Y558I:STX, respectively) (FIG. 3C). This structural change involves a repositioning of the carbamate carbon by 2 Å in the RcSxph-Y558I:STX complex relative to the RcSxph:STX complex. These findings are in line with the nearly equivalent toxin binding affinities of Y558A and Y558I, as well as with the idea that changes at the Tyr558 buttressing residue, Ile782, relieve the steric clash with STX. They also demonstrate that one strategy for increasing STX affinity is to engineer a highly organized binding pocket that requires minimal conformational changes to bind STX.

TABLE S1
Crystallographic data collection and refinement statistics

| | | | RcSxph - | | | | RcSxph - | |
|---|---|---|---|---|---|---|---|---|
| | RcSxph - | | Y588A:STX | | RcSxph - | | Y558I:STX | |
| | Y558A | | (co-crystal) | | Y558I | | (co-crystal) | |
| Data Collection | | | | | | | | |
| Space group | P2₁2₁2₁ | | P2₁2₁2₁ | | P2₁2₁2₁ | | P2₁2₁2₁ | |
| Cell dimensions | 96.61, 109.05, 254.89 | | 95.98, 107.14, 253.04 | | 96.39, 107.15, 254.79 | | 96.03, 107.81, 253.58 | |
| a/b/c (Å) | | | | | | | | |
| □/□/□ □(°) | 90, 90, 90 | | 90, 90, 90 | | 90, 90, 90 | | 90, 90, 90 | |
| Resolution (Å) | 47.81-2.60 | (2.65-2.60) | 47.37-2.60 | (2.65-2.60) | 47.61-2.70 | (2.76-2.70) | 47.5-2.15 | (2.19-2.15) |
| Rmerge (%) | 0.108 | (4.094) | 0.115 | (3.964) | 0.159 | (4.833) | 0.089 | (1.558) |
| I/σI | 12.9 | (0.9) | 12.5 | (0.8) | 8.8 | (0.6) | 16.2 | (1.2) |
| CC(1/2) | 0.998 | (0.532) | 0.998 | (0.458) | 0.998 | (0.419) | 0.999 | (0.599) |
| Completeness (%) | 99.6 | (100) | 99.9 | (100) | 99.9 | (99.8) | 99.5 | (93.6) |
| Redundancy | 13.4 | (13.9) | 13.4 | (14.0) | 13.3 | (14.0) | 12.2 | (6.2) |
| Total reflections | 1116931 | (62978) | 1085831 | (61318) | 975607 | (62994) | 1736282 | (40652) |
| Unique reflections | 83173 | (4517) | 81054 | (4377) | 73319 | (4504) | 142848 | (6596) |

| | | | | |
|---|---|---|---|---|
| Wilson B-factor | 83.42 | 84.83 | 90.34 | 44.01 |
| Wavelength (Å) | 1.033 | 1.033 | 1.033 | 1.033 |
| Refinement | | | | |
| R_{work}/R_{free} (%) | 22.79/26.37 | 23.20/26.17 | 23.42/27.21 | 20.73/23.37 |
| No. of chains in AU | 2 | 2 | 2 | 2 |
| No. of protein atoms | 12616 | 12616 | 12622 | 12622 |
| No. of ligand atoms | 0 | 42 | 0 | 72 |
| No. of water atoms | 77 | 60 | 79 | 794 |
| RMSD bond lengths (Å) | 0.002 | 0.002 | 0.003 | 0.003 |
| RMSD angles (°) | 0.50 | 0.49 | 0.55 | 0.59 |
| Ramachandran favored/ | 94.90/4.91/0.18 | 94.29/5.47/0.25 | 93.61/6.08/0.31 | 95.33/4.30/0.37 |
| allowed/outliers (%) | | | | |

TABLE S1
Crystallographic data collection and refinement statistics (continued)

| | RcSxph:F- | | | | NpSxph + STX | | NpSxph:F- | |
|---|---|---|---|---|---|---|---|---|
| | STX (soaked) | | NpSxph | | (co-crystal) | | STX (soaked) | |
| Data Collection | | | | | | | | |
| Space group | P2₁2₁2₁ | | R3 | | R3 | | R3 | |
| Cell dimensions | 96.44, 109.37, 256.36 | | 229.046, 229.046, 67.428 | | 228.848, 228.848, 67.224 | | 229.186, 229.186, 67.347 | |
| a/b/c (Å) | | | | | | | | |
| □/□/□ □(°) | 90, 90, 90 | | 90, 90, 120 | | 90, 90, 120 | | 90, 90, 120 | |
| Resolution (Å) | 47.97-2.65 | (2.70-2.65) | 43.29-2.2 | (2.279-2.2) | 39.88-2.0 | (2.071-2.0) | 42.62-2.2 | (2.279-2.2) |
| Rmerge (%) | 0.112 | (4.136) | 0.05218 | (0.8687) | 0.05456 | (0.9961) | 0.07078 | (1.889) |
| I/σI | 11.1 | (0.8) | 12.66 | (0.89) | 14.11 | (1.11) | 19.64 | (1.35) |
| CC(1/2) | 0.999 | (0.513) | 0.999 | (0.465) | 0.998 | (0.637) | 0.999 | (0.602) |
| Completeness (%) | 99.9 | (99.8) | 98.60 | (91.37) | 99.89 | (99.61) | 99.90 | (99.97) |
| Redundancy | 10.4 | (11.0) | 3.3 | (2.1) | 5.2 | (4.8) | 10.6 | (11.0) |
| Total reflections | 830262 | (49019) | 219806 | (12669) | 459480 | (42277) | 707304 | (73200) |
| Unique reflections | 79524 | (4469) | 66067 | (6127) | 88641 | (8856) | 66898 | (6682) |

| | | | | |
|---|---|---|---|---|
| Wilson B-factor | 83.73 | 52.73 | 49.89 | 55.11 |
| Wavelength (Å) | 1.033 | 1.033167 | 1.033167 | 1.033167 |
| Refinement | | | | |
| R_{work}/R_{free} (%) | 23.46/26.51 | 19.50/23.55 | 19.25/22.09 | 19.64/24.26 |
| No. of chains in AU | 2 | 1 | 1 | 1 |
| No. of protein atoms | 12630 | 6373 | 6385 | 6346 |
| No. of ligand atoms | 110 | 38 | 59 | 93 |
| No. of water atoms | 88 | 229 | 287 | 161 |
| RMSD bond lengths (Å) | 0.002 | 0.004 | 0.004 | 0.006 |
| RMSD angles (°) | 0.50 | 0.60 | 0.62 | 0.75 |
| Ramachandran favored/ | 94.23/5.41/0.37 | 95.71/4.17/0.12 | 95.85/3.90/0.24 | 94.19/5.56/0.25 |
| allowed/outliers (%) | | | | |

Sxph STX Binding Affinity Changes Alter Na_{V} Rescue from STX Block

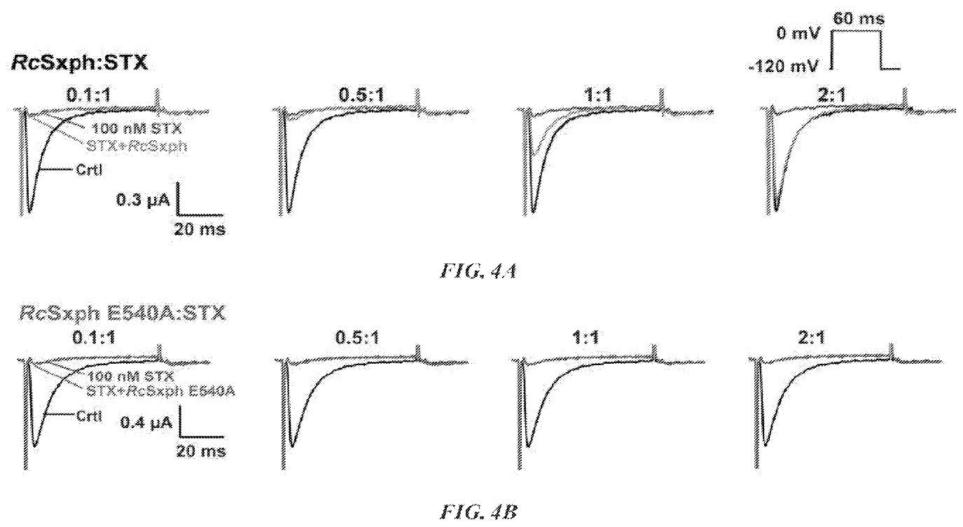

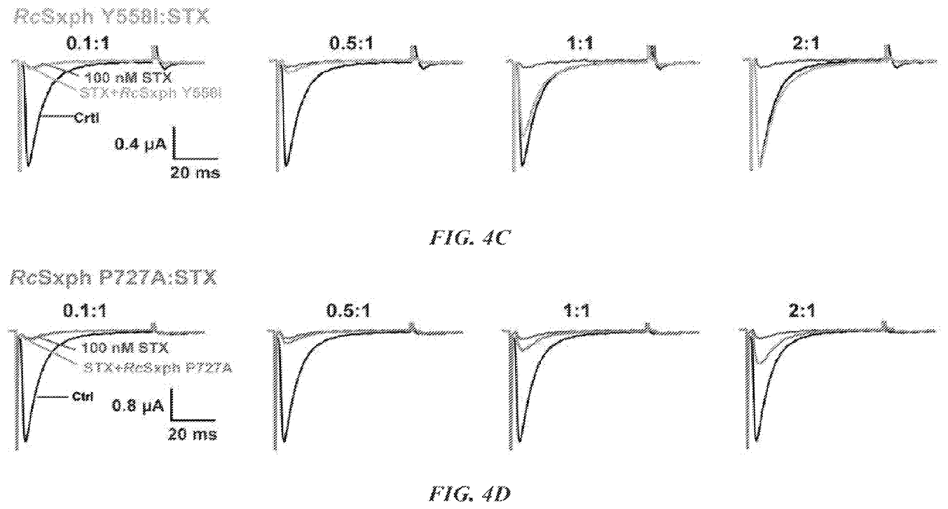

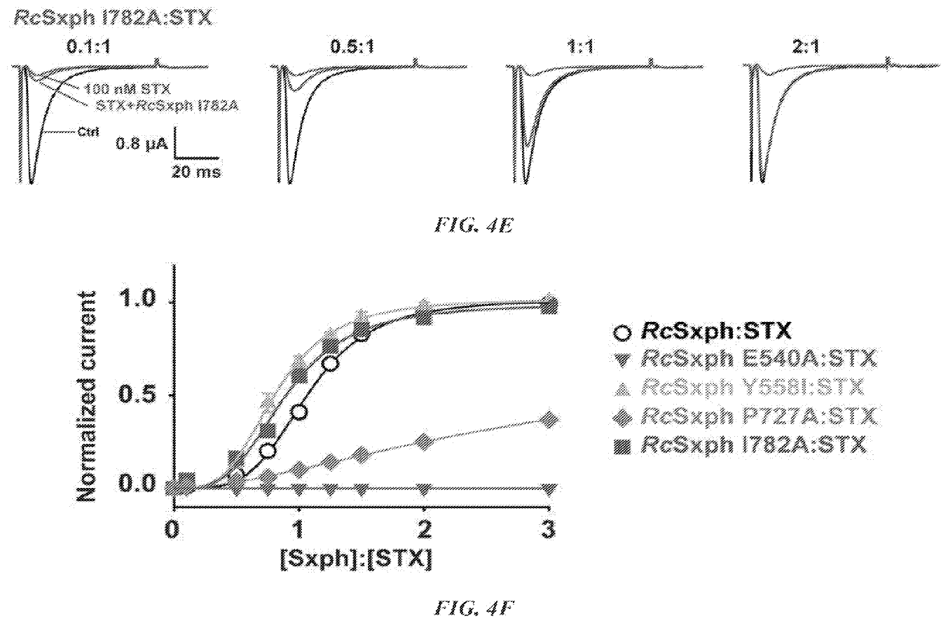

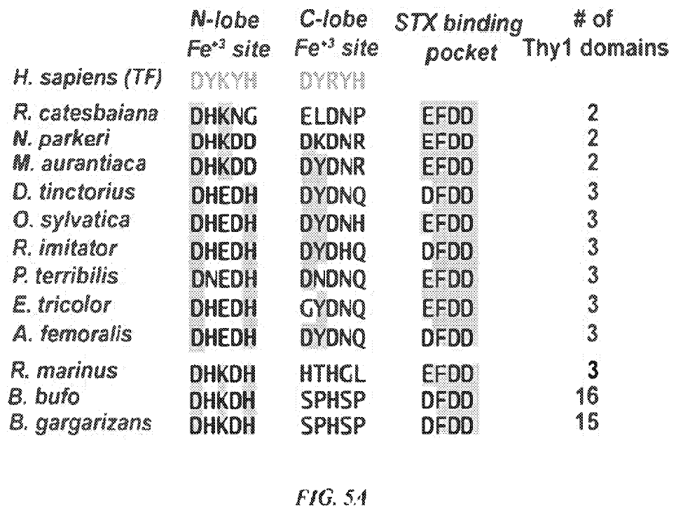

RcSxph acts as a ‘toxin sponge’ that can reverse STX inhibition of Na_{V}s (Abderemane-Ali et al.). To test the extent to which this property is linked to the intrinsic affinity of RcSxph for STX, we evaluated how STX affinity altering mutations affected RcSxph rescue of channels blocked by STX. As shown previously, titration of different RcSxph:STX ratios against Phyllobates terribilis Na_{V}1.4 (PtNa_{V}1.4), a Nav having an IC₅₀ for STX of 12.6 nM (Abderemane-Ali et al.), completely reverses the effects of STX at ratios of 2:1 RcSxph:STX or greater (FIGS. 4A and F). Incorporation of mutations that affect STX affinity altered the ability of RcSxph to rescue Na_{V}s and followed the binding assay trends. Mutants that increased STX affinity, Y558I and I782A, improved the ability of RcSxph to rescue PtNa_{V}1.4 (Effective Rescue Ratio₅₀ (ERR₅₀)=0.81±0.01, 0.87±0.02, and 1.07±0.02 for Y558I, I782A, and RcSxph, respectively), whereas mutations that compromised STX binding reduced (P727A, ERR₅₀>4) or eliminated (E540A) the ability of RcSxph to reverse the STX inhibition (FIG. 4B-F). This strong correlation indicates that the ‘toxin sponge’ property of Sxph (Abderemane-Ali et al.) depends on the capacity of Sxph to sequester STX and adds further support to the idea that Sxph has a role in toxin resistance mechanisms (Mahar et al., Abderemane-Ali et al.).

### Expansion of the Sxph Family

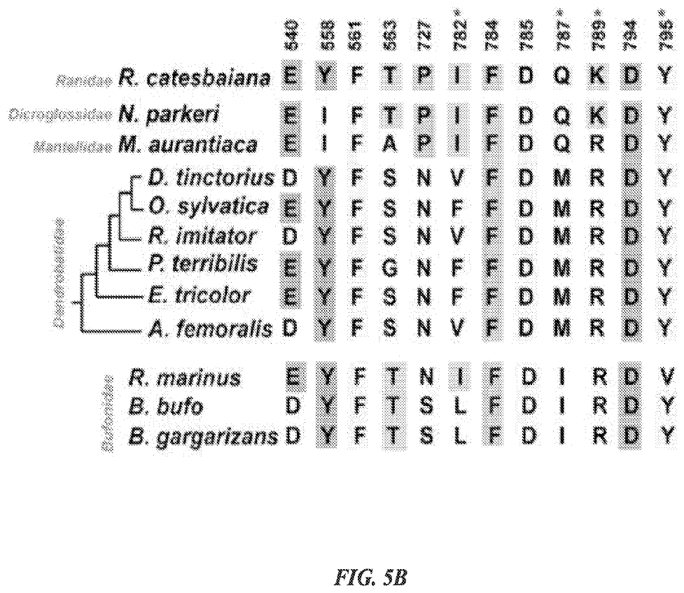

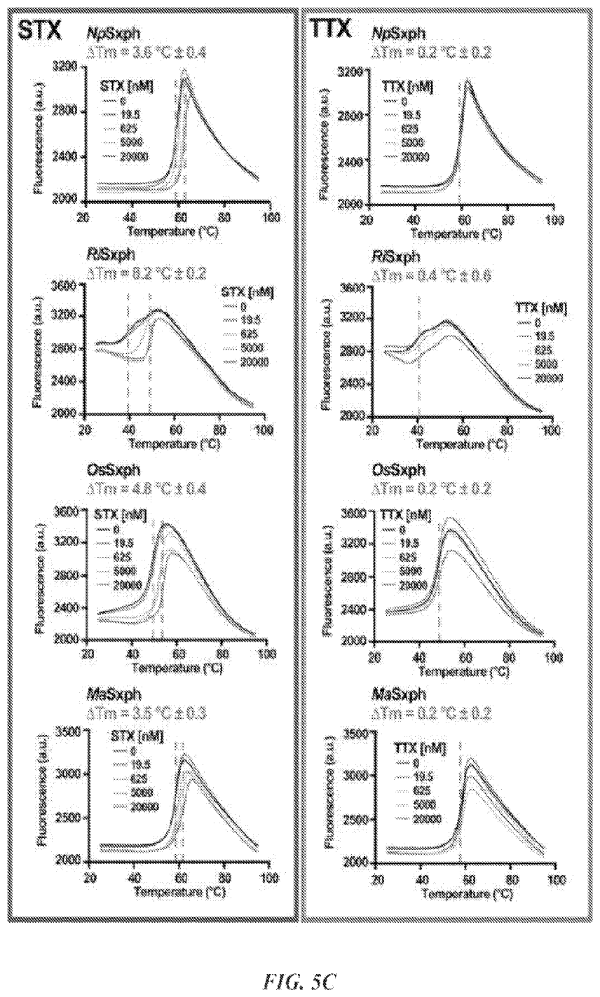

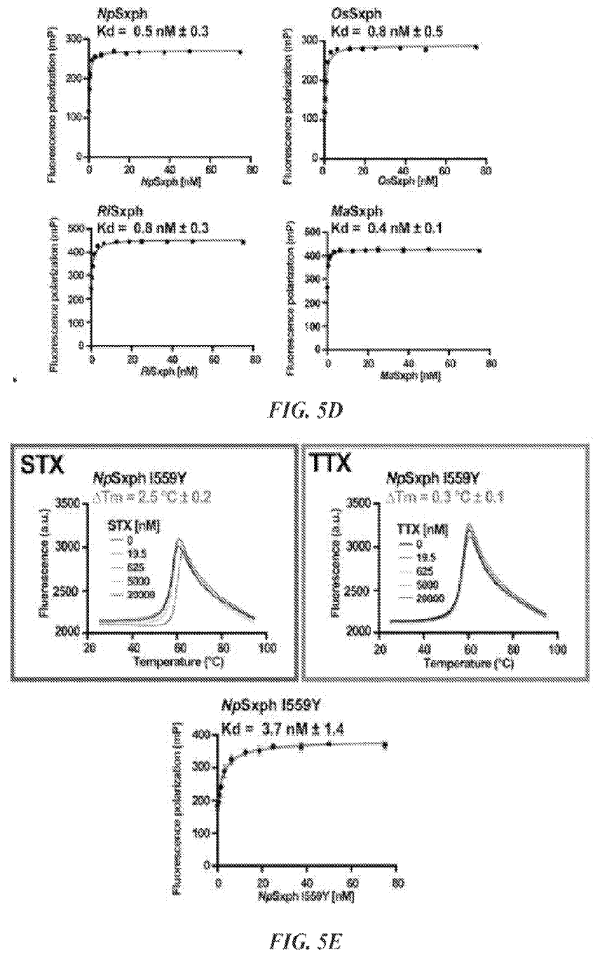

STX binding activity has been reported in the plasma, hemolymph, and tissues of diverse arthropods, amphibians, fish, and reptiles (Doyle et al., Llewellyn et al.), suggesting that many organisms harbor Sxph-like proteins. Besides RcSpxh, similar Sxphs have been identified in only two other frogs, the High Himalaya frog Nanorana parkeri (Yen et al.) and the little devil poison frog Oophaga sylvatica (Caty et al.). As a number of poison frogs exhibit resistance to STX poisoning (Abderemane-Ali et al.), we asked whether the STX binding site ‘recognition code’ could enable identification of Sxph homologs in other amphibians. To this end, we determined the sequences of ten new Sxphs (FIGS. 5A and B, FIG. S7, and FIG. S8). These include six Sxphs in two poison dart frog families (Family Dendrobatidae: Dyeing poison dart frog, Dendrobates tinctorius; Little devil poison frog, O. sylvatica; Mimic poison frog, Ranitomeya imitator; Golden dart frog, Phyllobates terribilis; Phantasmal poison frog, Epipedobates tricolor; and Brilliant-thighed poison frog, Allobates femoralis; and Family: Mantellidae Golden mantella, Mantella aurantiaca), and three Sxphs in toads (Caucasian toad, Bufo; Asiatic toad, Bufo gargarizans; and South American cane toad, Rhinella marina). The identification of the OsSxph sequence confirms its prior identification by mass spectrometry (Caty et al.) and the discovery of RmSxph agrees with prior reports of Sxph-like STX binding activity in the cane toad (R. marinus) (Llewellyn et al., Tanaka et al.).

Sequence comparisons (FIGS. S7 and S8) show that all of the new Sxphs share the transferrin fold found in RcSxph comprising N- and C-lobes each having two subdomains (N1, N2 and C1, C2, respectively) (Yen et al., Morabito and Moczdlowski) and the signature ‘EFDD’ motif (Yen et al.) or a close variant in the core of the C-lobe STX binding site (FIG. 5A). Similar to RcSxph, the new Sxphs also have amino acid differences relative to transferrin that should eliminate Fe³⁺ binding (Yen et al., Morabito and Moczdlowski, Li et al.), as well as a number of protease inhibitor thyroglobulin domains (Thy1) inserted between the N1 and N2 N-lobe subdomains (Yen et al., Lenarčič et al.) (FIG. 5A and FIGS. S7-S9). These Thy1 domain insertions range from two in RcSxph, NpSxph, and MaSxph, to three in the dendrobatid poison frog and cane toad Sxphs, to 16 and 15 in toad BbSxph and BgSxph, respectively (FIG. 5A and FIGS. S7-S9).

We used the STX recognition code defined by our studies as a template for investigating cross-species variation in the residues that contribute to STX binding (FIG. 5B). This analysis shows a conservation of residues that interact with the STX bis-guanidium core (Glu540, Phe784, Asp785, Asp794, and Tyr795) and carbamate (Phe561). Surprisingly, five of the Sxphs (D. tinctorius, R. imitator, A. femoralis, B. bufo, and B. gargarizans) have an aspartate instead of a glutamate at the Glu540 position in RcSxph that contributes the most binding energy (FIG. 2A). The equivalent change in RcSxph, E540D, reduced STX affinity by ˜100 fold (Table 1) and uniquely alters enthalpy and entropy binding parameters compared to other affinity-lowering mutations (Table S2). Additionally, we identified variations at two sites for which mutations increase RcSxph STX binding, Tyr558 and Ile782 (FIGS. 2A, 4C, 4E, and Table 1). NpSxph and MaSxph have an Ile at the Tyr558 site, whereas eight of the new Sxphs have hydrophobic substitutions at the Ile782 position (FIG. 5B). The striking conservation of the Sxph scaffold and STX binding site indicate that this class of ‘toxin sponge’ proteins is widespread among diverse Anurans, while the amino acid variations in key positions (Glu540, Tyr558, and Ile782), raise the possibility that the different Sxph homologs have varied STX affinity or selectivity for STX congeners.

TABLE S2
RcSxph:STX and NpSxph:STX thermodynamic binding parameters

| | | | | □H | □S | □G | |
|---|---|---|---|---|---|---|---|
| | | N | Kd | (kcal | (cal mol⁻¹ | (kcal | |
| | | (sites) | (nM) | mol⁻¹) | K⁻¹) | mol⁻¹) | n |
| RcSxph | WT | 1.02 ± 0.01 | 1.2 ± 0.8 | −16.1 ± 0.2 | −12.7 ± 0.9 | −12.3 ± 0.5 | 3 |
| | Y558A | 1.01 ± 0.01 | 1.2 ± 0.4 | −15.3 ± 0.0 | −11.1 ± 1.1 | −12.2 ± 0.2 | 2 |
| | Y558I | 1.05 ± 0.03 | 1.1 ± 0.5 | −15.5 ± 0.3 | −10.5 ± 0.6 | −12.2 ± 0.3 | 2 |
| | F561A | 1.07 ± 0.01 | 13.4 ± 1.4 | −12.7 ± 0.2 | −6.6 ± 0.9 | −10.8 ± 0.1 | 3 |
| | P727A | 0.97 ± 0.03 | 31.3 ± 11.6 | −11.5 ± 0.0 | −4.2 ± 0.9 | −10.3 ± 0.2 | 2 |
| | E540D | 0.98 ± 0.02 | 68.9 ± 10.7 | −16.3 ± 1.7 | −21.9 ± 5.7 | −9.8 ± 0.1 | 4 |
| | D794E | 0.99 ± 0.01 | 312.5 ± 2.9 | −11.8 ± 0.0 | −8.9 ± 0.1 | −8.9 ± 0.1 | 2 |
| NpSxph | WT | 0.92 ± 0.02 | 2.5 ± 0.1 | −18.7 ± 0.2 | −23.2 ± 0.8 | −11.8 ± 0.1 | 2 |
| | I559Y | 0.94 ± 0.03 | 2.5 ± 0.8 | −16.8 ± 0.2 | −16.9 ± 1.1 | −11.8 ± 0.2 | 4 |

Diverse Sxph Family Members have Conserved STX Binding Properties

To explore the STX binding properties of this new set of Sxphs and to begin to understand whether changes in the binding site composition affect toxin affinity, we expressed and purified four representative variants. These included two Sxphs having STX binding site sequences similar to RcSxph (NpSxph and MaSxph) and two Sxphs bearing more diverse amino acid differences (RiSxph and OsSxph), including one displaying the E540D substitution (RiSxph). This set also represents Sxphs having either two Thy1 domains similar to RcSxph (NpSxph and MaSxph) or three Thy1 domains (OsSxph and RiSxph) (FIG. 5A). TF experiments showed STX-dependent DTms for all four Sxphs. By contrast, equivalent concentrations of TTX had no effect (FIG. 5C), indicating that, similar to RcSpxh (FIG. 1A) (Mahar et al., Abderemane-Ali et al.), all four Sxphs bind STX but not TTX. Unlike the other Sxphs, the RiSxph melting curve showed two thermal transitions; however, only the first transition was sensitive to STX concentration (FIG. 5C). FP binding assays showed that all four Sxphs bound F-STX and revealed affinities stronger than RcSxph (FIG. 5D and Table 2). The enhanced affinity of NpSxph and MaSxph for STX relative to RcSxph is consistent with the presence of the Y558I variant (FIG. 5B). Importantly, the observation that RiSxph has a higher affinity for STX than RcSxph despite the presence of the E540D difference suggests that the other sequence variations in the RiSxph STX binding pocket compensate for this Glu→Asp change at Glu540.

TABLE 2
Comparison of Sxph STX binding properties

| Construct | Kd (nM) | □□G (kcal mol⁻¹) | n |
|---|---|---|---|
| MaSxph | 0.4 ± 0.1 | −1.73 | 4 |
| NpSxph | 0.5 ± 0.3 | −1.60 | 13 |
| RiSxph | 0.8 ± 0.3 | −1.32 | 4 |
| OsSxph | 0.8 ± 0.5 | −1.32 | 10 |
| NpSxph I559Y | 3.7 ± 1.4 | −0.04 | 8 |
| RcSxph | 7.4 ± 2.6 | 0 | 10 |

Kd, dissociation constant;
n, number of observations
□□G = RT ln (Kd(XSxph)/Kd(RcSxph)); T = 298K.
Errors for measurements are S.D.

Because NpSxph has a higher affinity for STX than RcSxph (FIGS. 1D, 5D and Table 2) and has an isoleucine at the Tyr558 site (FIG. 5B), we asked whether the NpSxph I559Y mutant that converts the NpSxph binding site to match RcSxph would lower STX affinity. TF measurements showed that NpSxph I559Y had a ˜1° C. smaller DTm than NpSxph (DTm=3.6° C.±0.4 versus 2.5° C.±0.2 for NpSxph and NpSxph I559Y, respectively), indicative of a decreased binding affinity (FIGS. 5C and E). This result was validated by FP (DDG=−1.56 kcal mol⁻¹), yielding a result of similar magnitude to the RcSxph Y558I differences (FIG. 5E, Tables 1 and 2). ITC confirmed the high affinity of the interaction (FIG. S5D-F), but could not yield an explicit Kd given its low nanomolar value (FIG. S5F). Nevertheless, these experiments validate the 1:1 stoichiometry of the STX:NpSTX interaction (Table S2) and show that the I559Y change reduced the binding enthalpy, consistent with perturbation ofNpSxph:STX interactions (DH=−18.7±0.2 vs. −16.8±0.2 kcal mol⁻¹, NpSxph and NpSxph I559Y, respectively) (Table S2). Taken together, these experiments establish the conserved nature of the STX binding pocket among diverse Sxph homologs and show that the STX recognition code derived from RcSxph studies (FIG. 5B) can identify key changes that influence toxin binding.

### Structures of Apo- and STX Bound NpSxph Reveal a Pre-Organized STX Binding Site

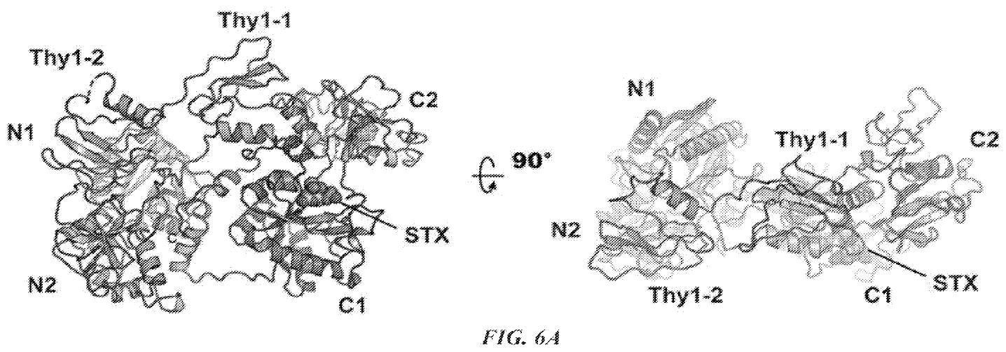

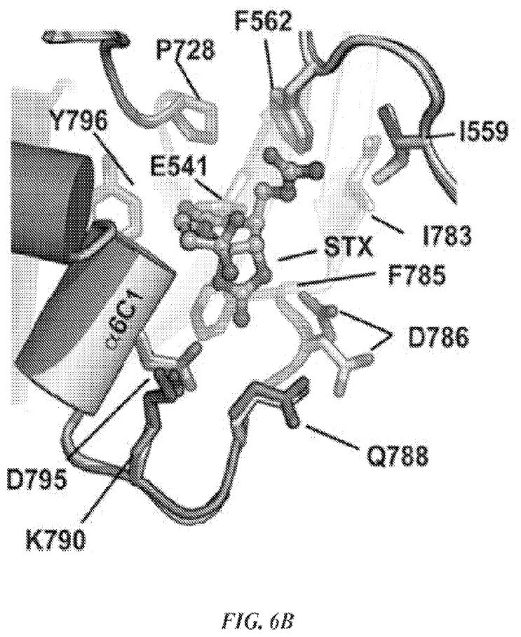

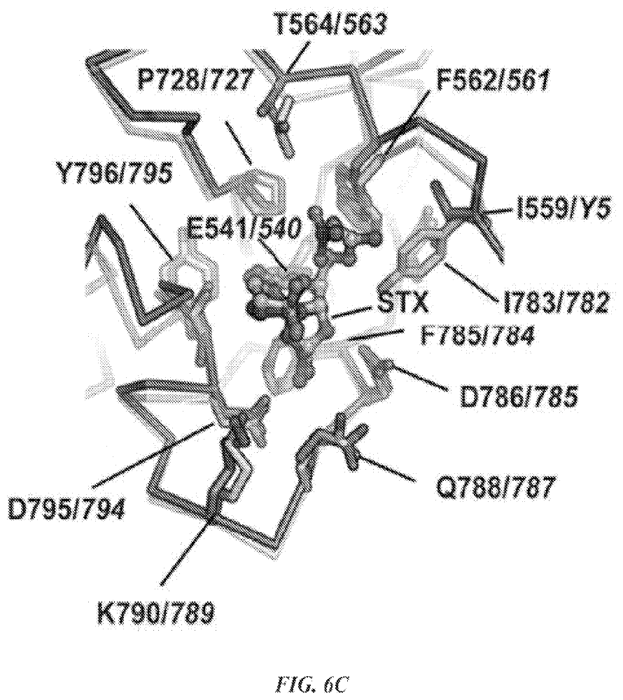

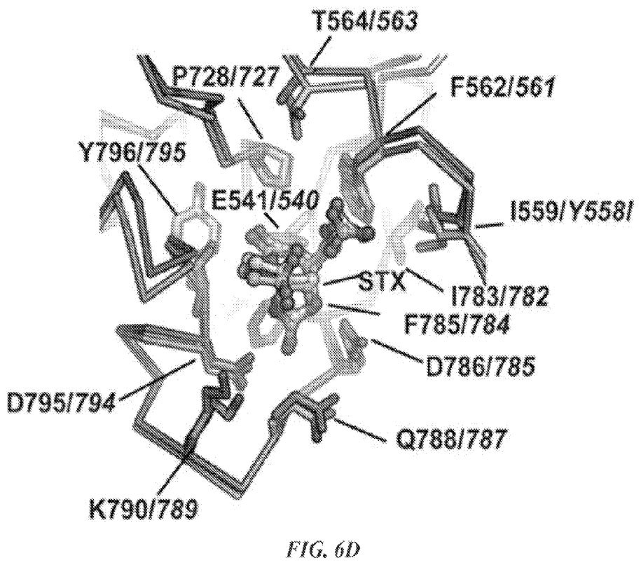

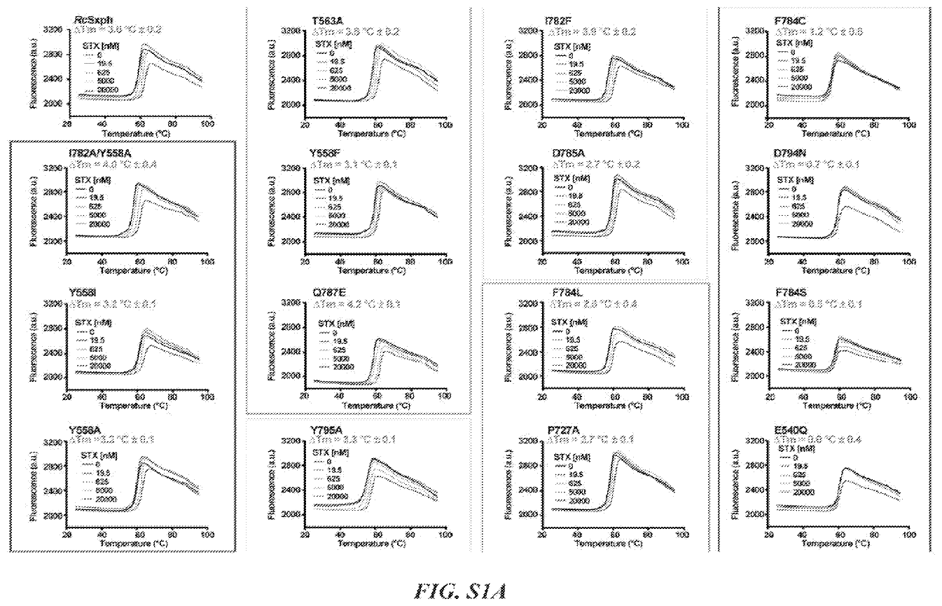

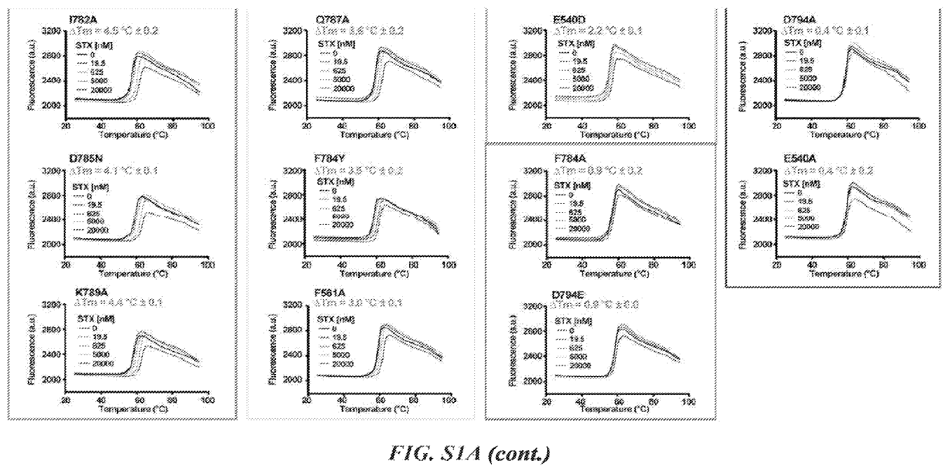

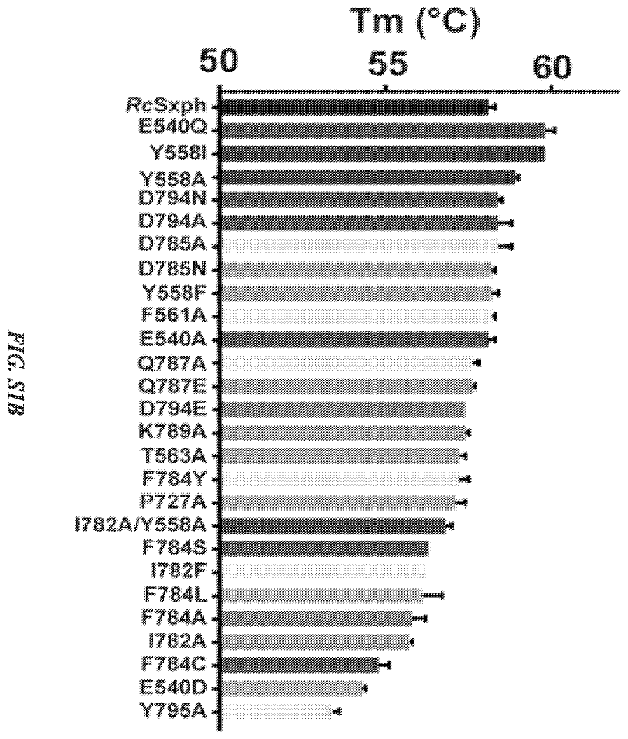

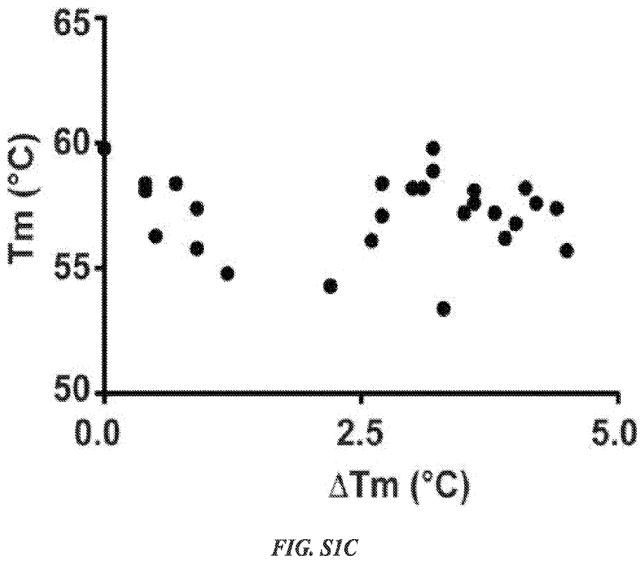

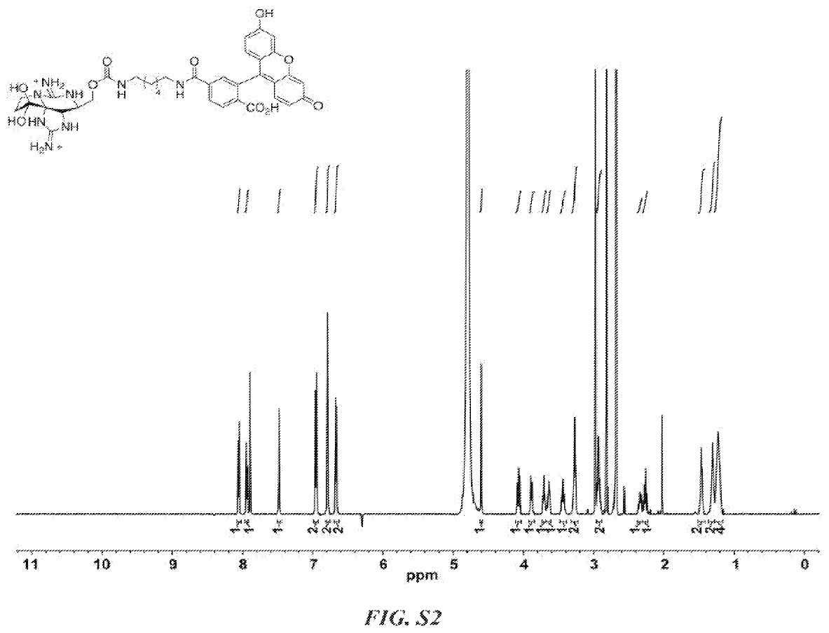

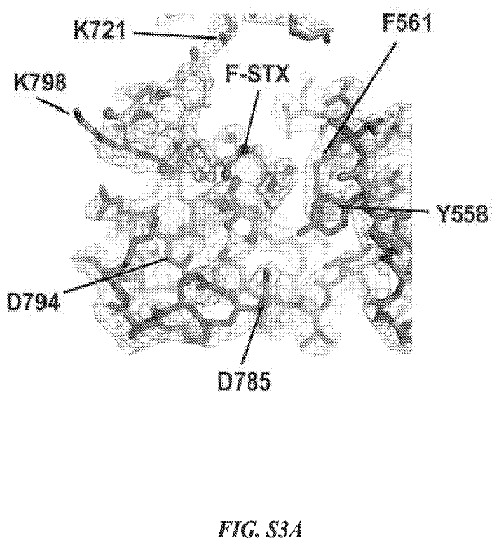

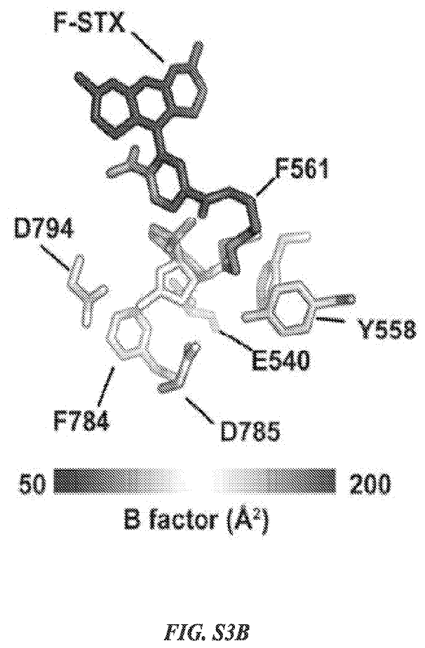

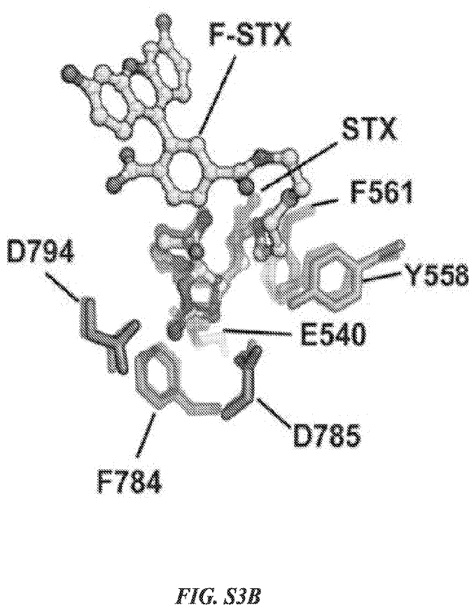

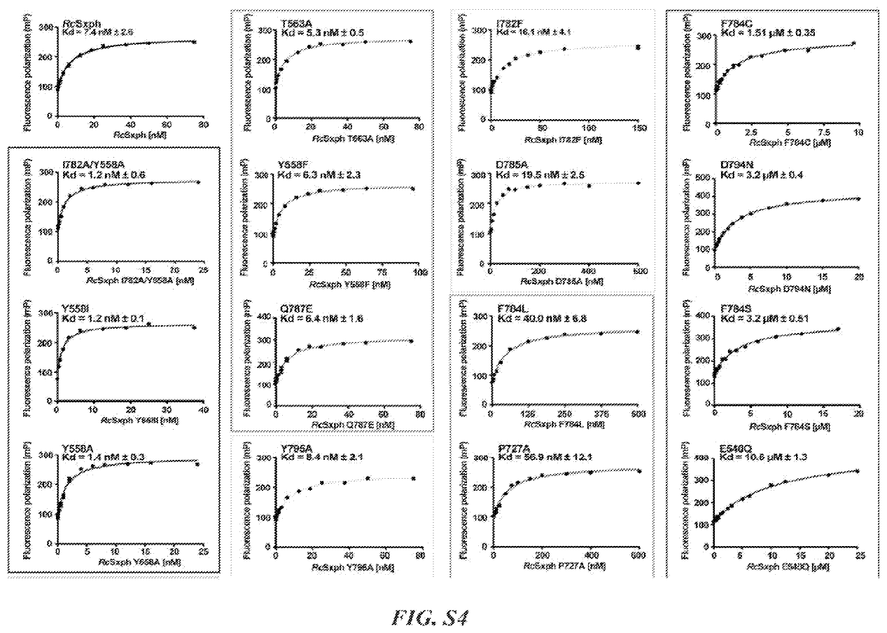

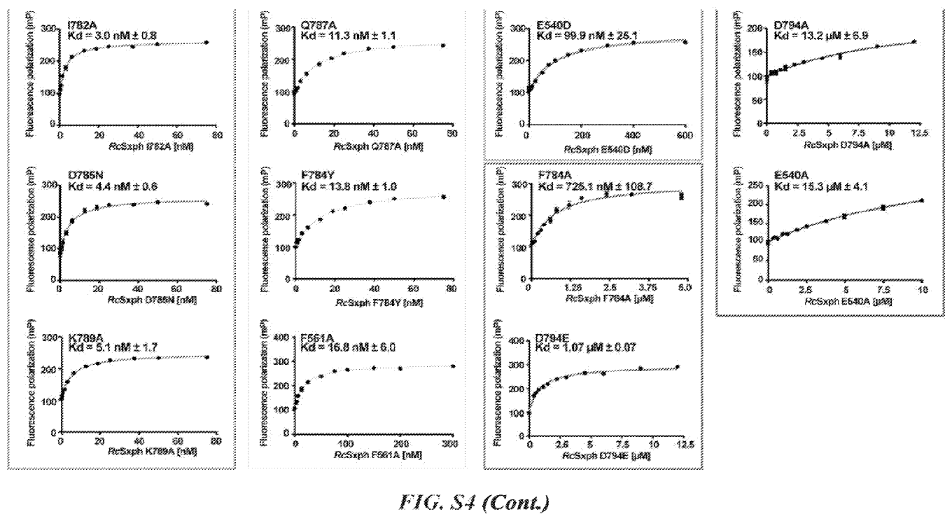

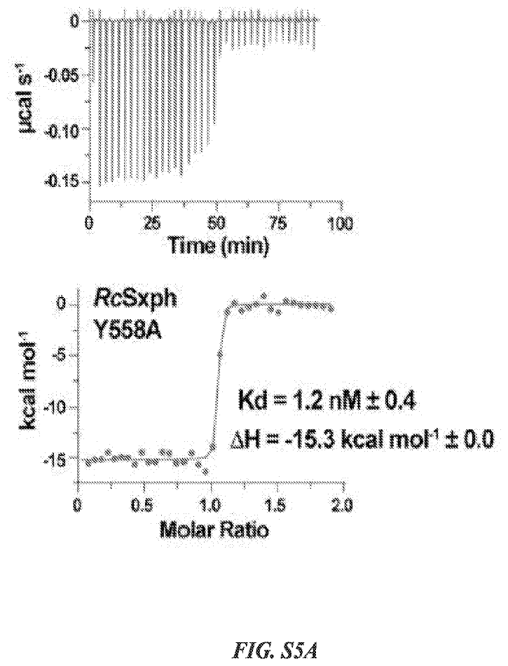

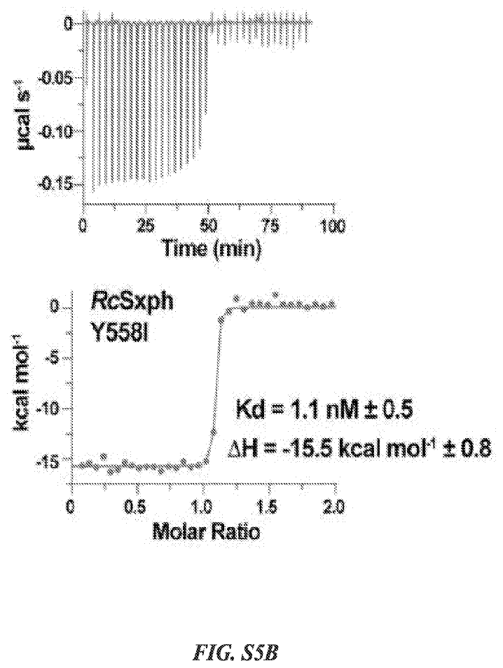

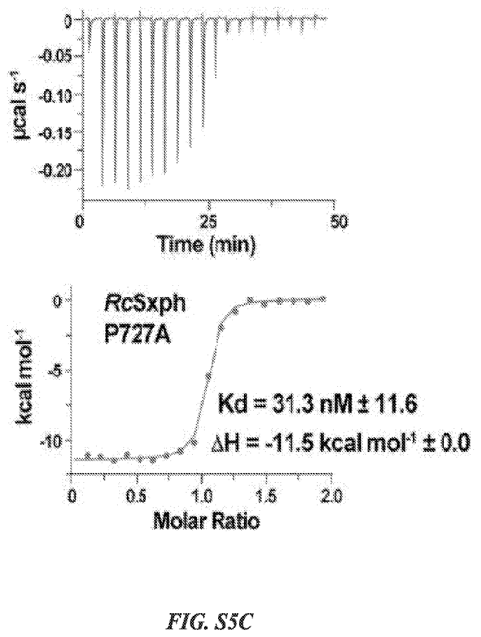

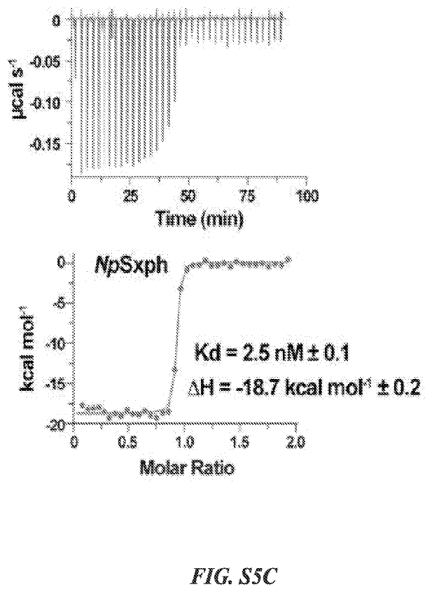

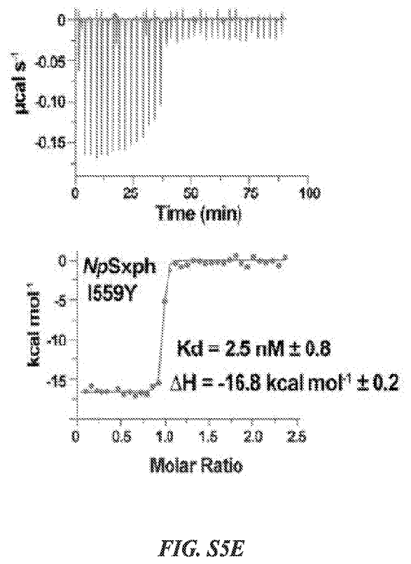

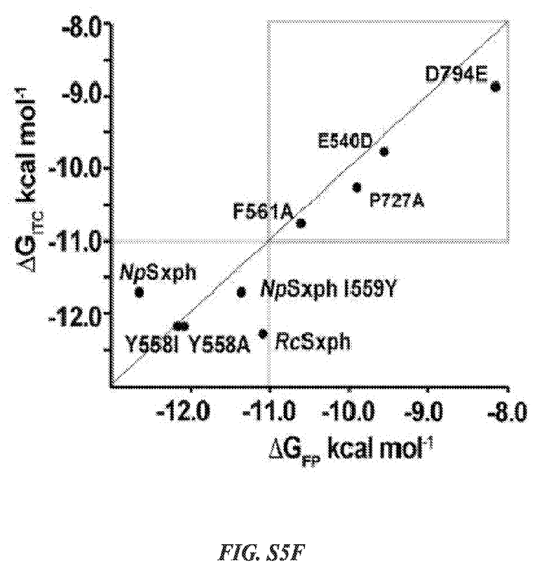

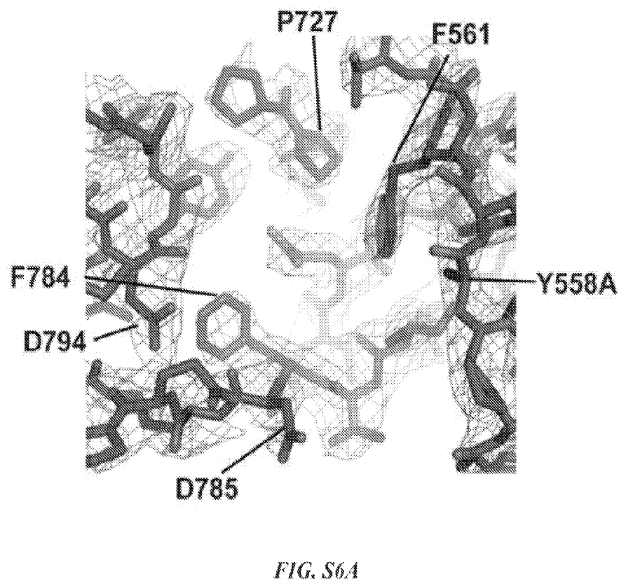

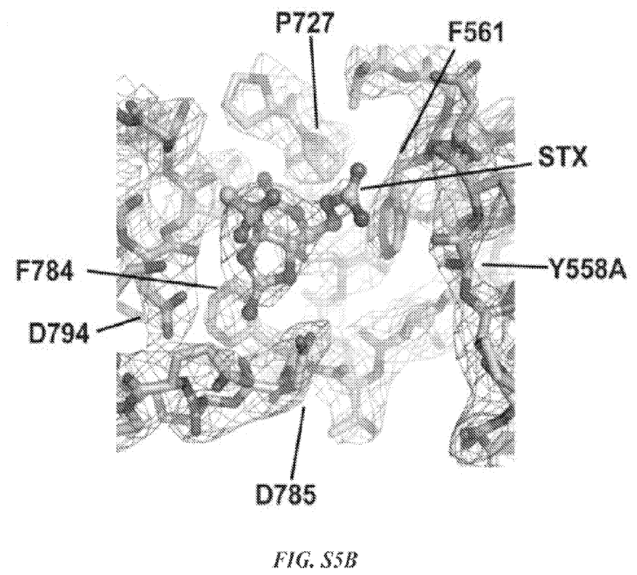

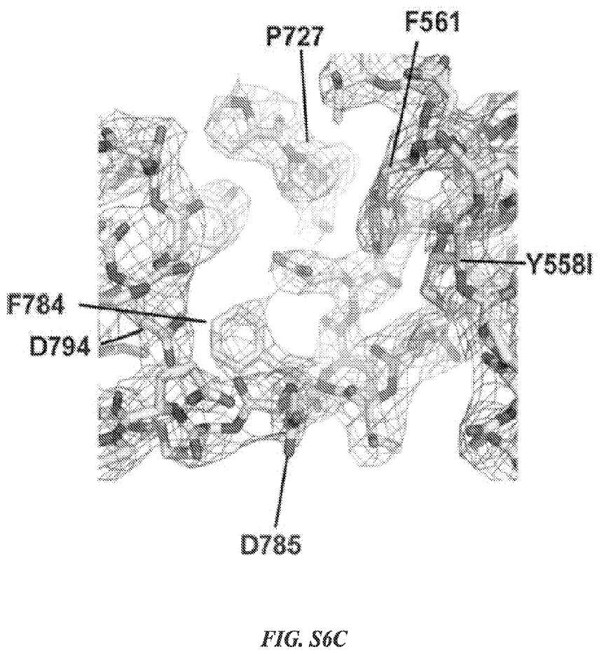

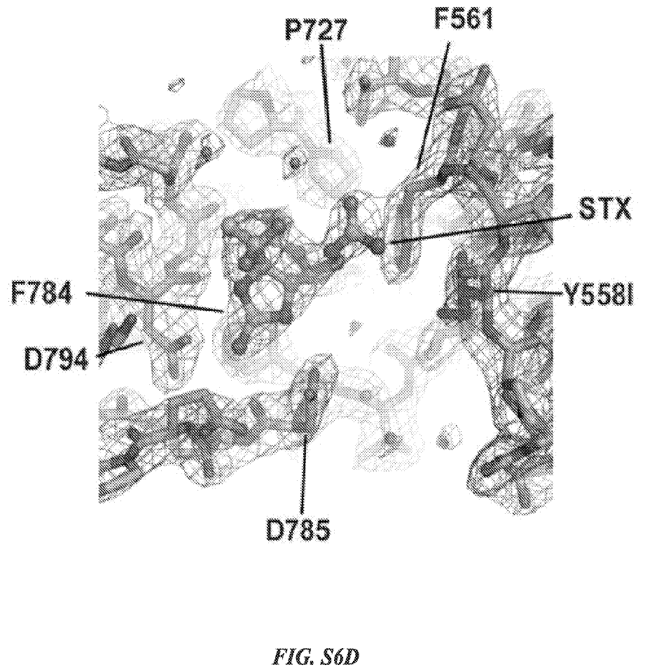

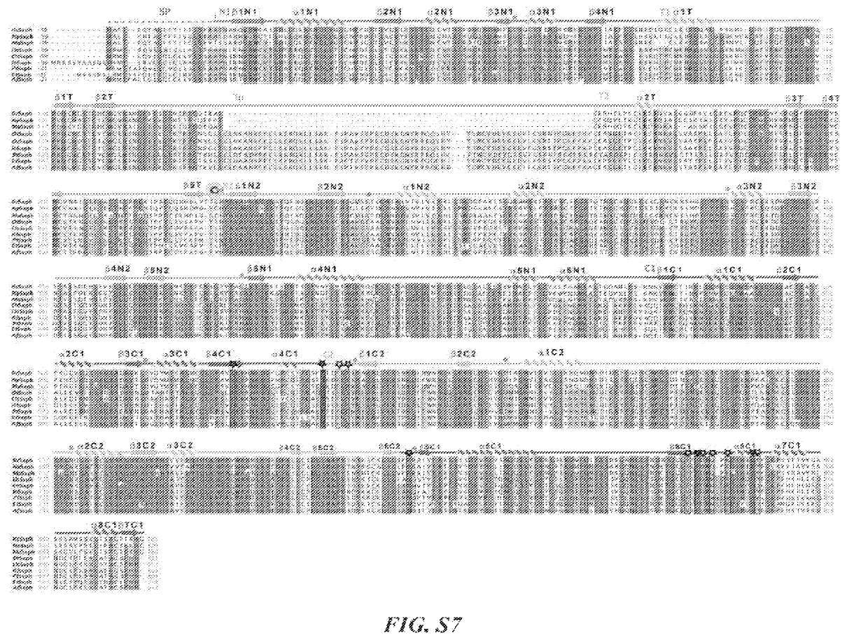

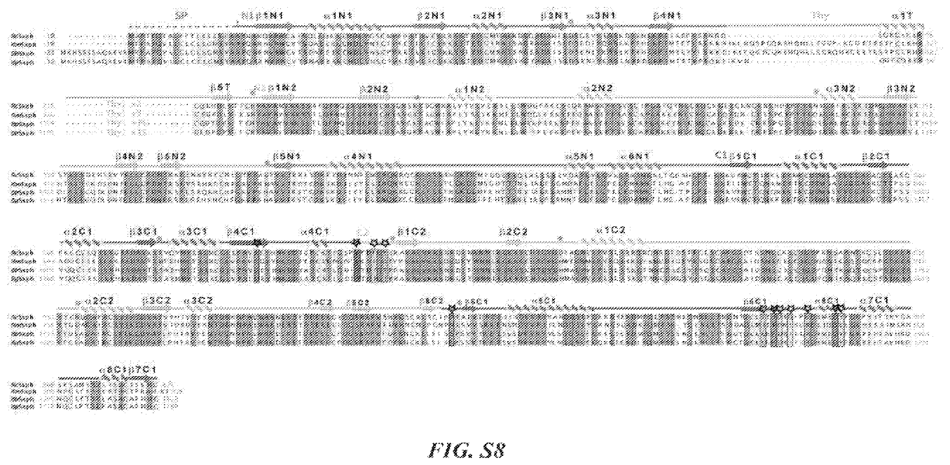

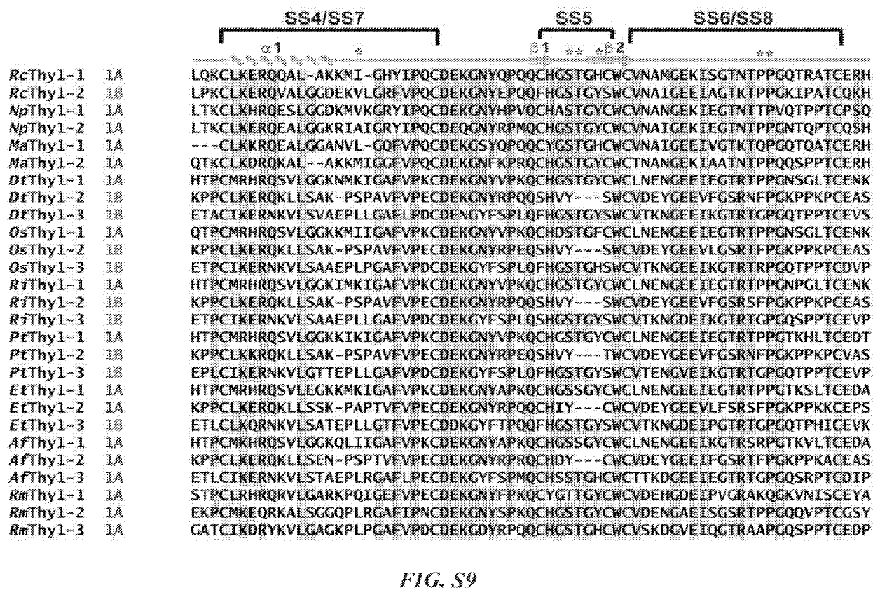

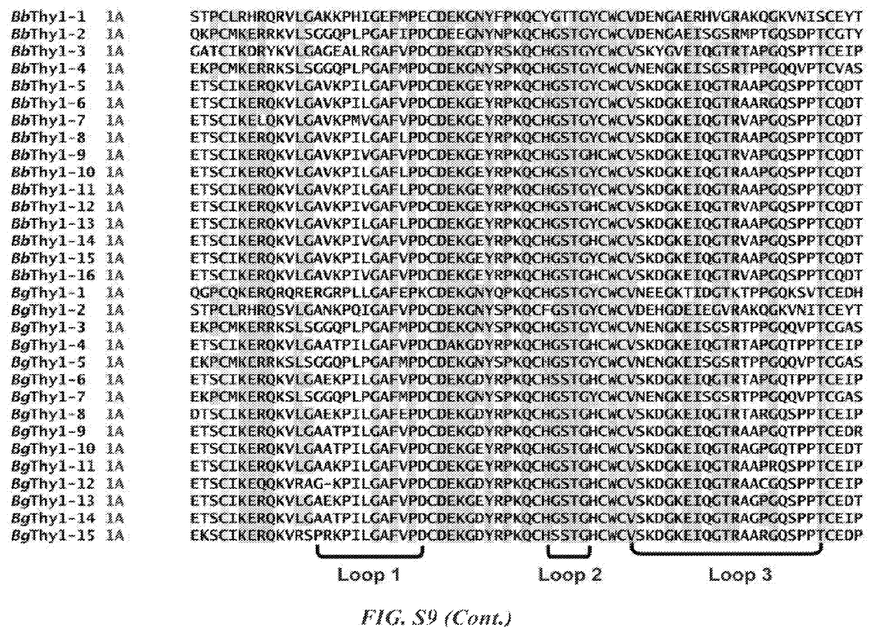

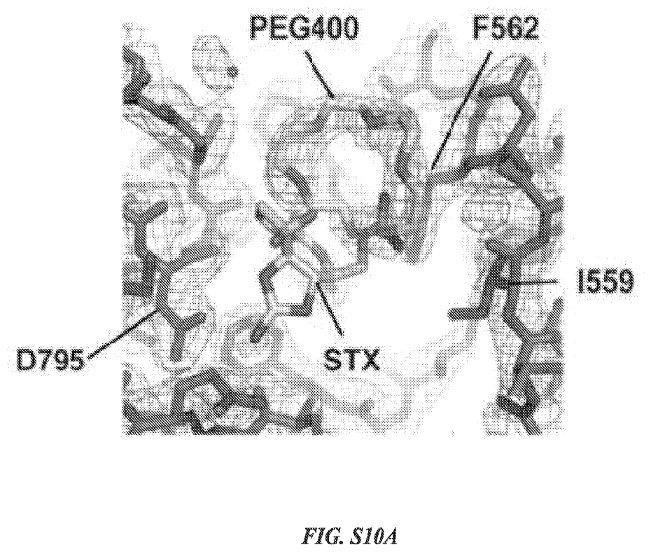

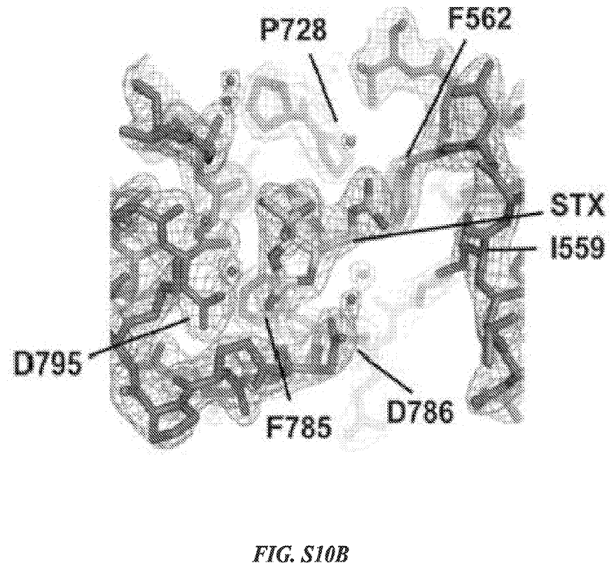

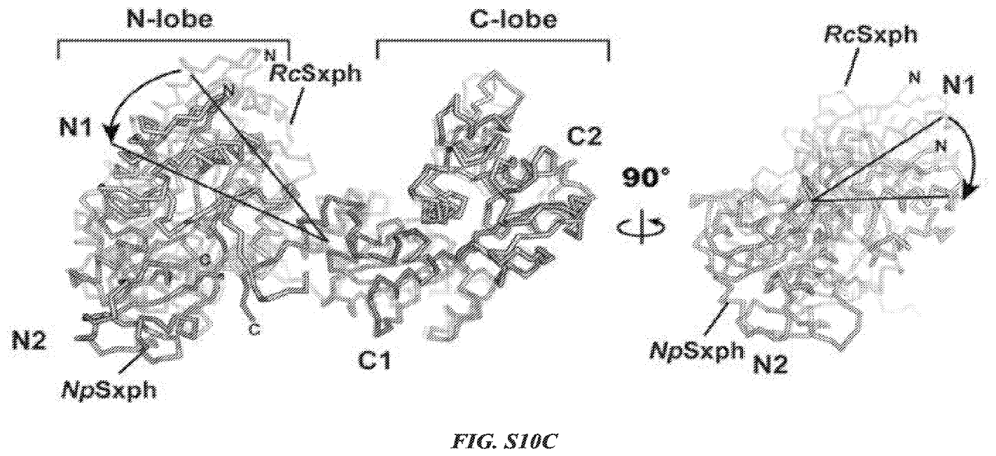

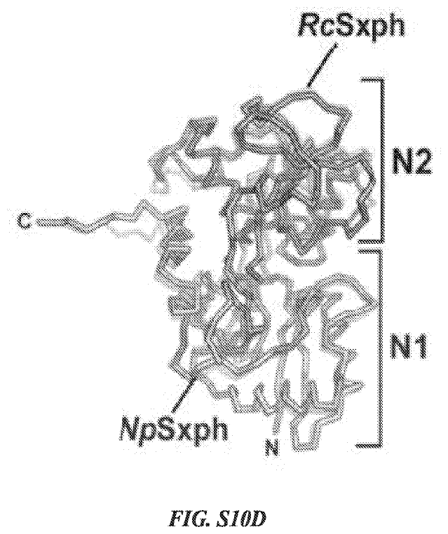

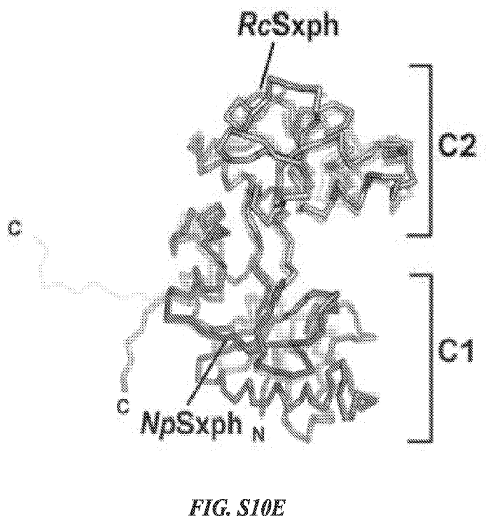

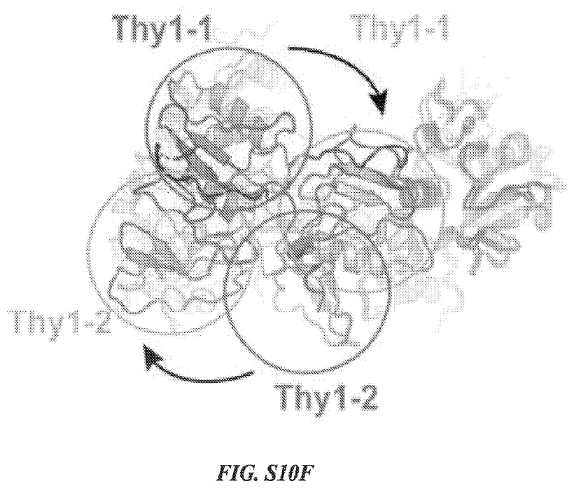

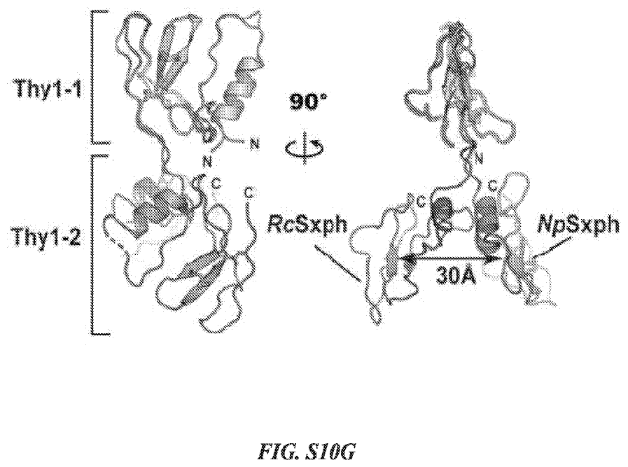

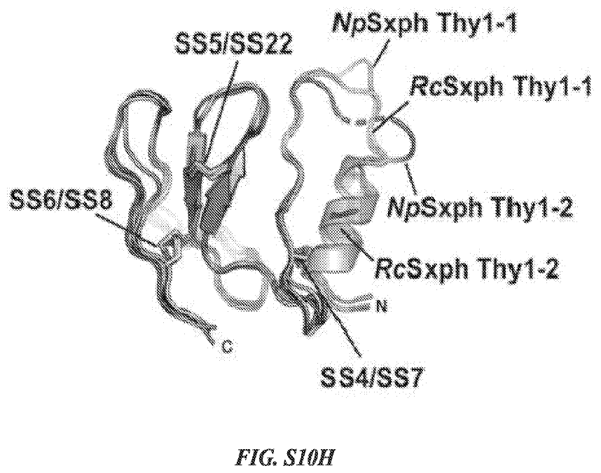

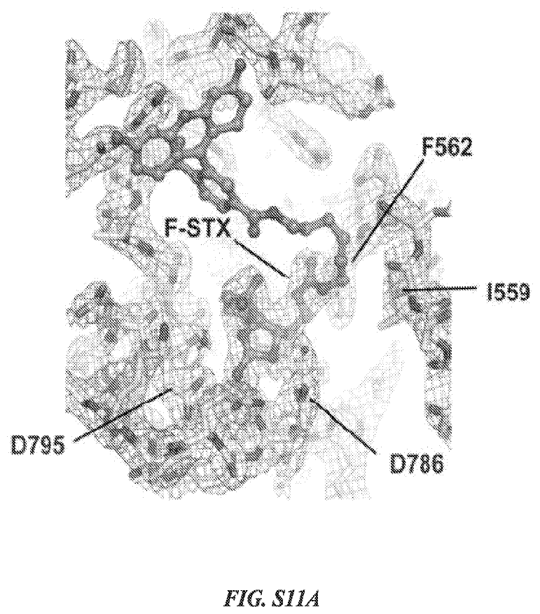

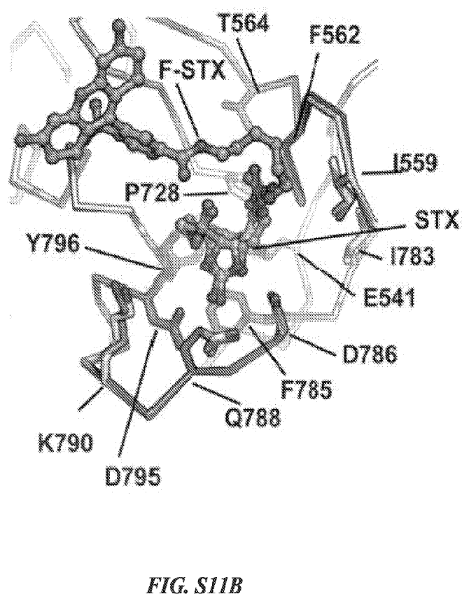

We crystallized and determined the structure of NpSxph, alone and co-crystallized with STX to compare STX binding modes among Sxph family members. NpSxph and STX:NpSxph crystals diffracted X-rays to resolutions of 2.2 Å and 2.0 Å, respectively, and were solved by molecular replacement (FIGS. 6A, S10A and B). As expected from the similarity to RcSxph, NpSxph is built on a transferrin fold (FIG. 6A) and has the same 21 disulfides found in RcSxph, as well as an additional 22^{nd} disulfide in the Type 1 Å thyroglobulin domain of NpSxph Thy1-2. However, structural comparison of NpSxph and RcSxph reveals a number of unexpected large-scale domain rearrangements.

The NpSxph N-lobe is displaced along the plane of the molecule by ˜30° and rotated around the central axis by a similar amount (FIG. S10C). NpSxph N-lobe and C-lobe lack Fe³⁺ binding sites (FIG. 5A), and despite the N-lobe displacement relative to RcSxph adopt closed and open conformations, respectively as in RcSxph (Yen et al.) (FIG. S10D-E) (RMSD_{Ca}=1.160 Å and 1.373 Å for NpSxph and RcSxph N- and C-lobes, respectively). Surprisingly, the two NpSxph Thy1 domains are in different positions than in RcSxph and appear to move as a unit by ˜90° with respect to the central transferrin scaffold (FIG. S10F and Supplementary movie M3) and a translation of ˜30 Å of Thy1-2 (FIG. S10G). Thy1-1 is displaced from a site over the N-lobe in RcSxph to one in which it interacts with the NpSxph C-lobe C2 subdomain and Thy1-2 moves from between the N and C-lobes in RcSxph where it interacts with the C1 subdomain, to a position in NpSxph where it interacts with both N-lobe subdomains. Consequently, the interaction between the C-lobe b-strand b7C1 and Thy1-2 b5 observed in RcSxph is absent in NpSxph. Despite these domain-scale differences, Thy1-1 and Thy1-2 are structurally similar to each other (RMSD_{Ca}=1.056 Å) and to their RcSxph counterparts (FIG. S10H) (RMSD_{Ca}=1.107 Å and 0.837, respectively). Further, none of these large scale changes impact the STX binding site, which is found on the C1 domain as in RcSxph (FIG. 5A).

Comparison of the apo- and STX-bound NpSxph structures shows that there are essentially no STX binding site conformational changes upon STX engagement, apart from the movement of Asp786 to interact with the STX five-membered guanidinium ring (FIG. 6B and Supplementary Movie M4). This conformational change is shared with RcSxph (Yen et al.) and appears to be a common element of Sxph binding to STX. The movements of Tyr558 and its loop away from the STX binding site observed in RcSxph (Yen et al.) are largely absent in NpSxph for the Tyr558 equivalent position (Ile559) and its supporting loop. Hence, the NpSxph STX binding site is better organized to accommodate STX (FIG. 6B), similar to RcSxph Y558I (FIG. 3B). We also noted an electron density in the apo-NpSxph STX binding site that we assigned as a PEG400 molecule from the crystallization solution (FIG. S10A). This density occupies a site different from STX and is not present in the STX-bound complex (FIG. S10B) and suggests that other molecules may be able to bind the STX binding pocket.

We also determined the structure of an NpSxph:F-STX complex at 2.2 Å resolution (Table S1). This structure shows no density for the fluorescein moiety and has an identical STX pose to the NpSxph:STX complex (FIG. S11), providing further evidence that fluorescein does not interact with Sxph (cf. FIG. S3) even though it is tethered to the STX binding pocket and the FP assay faithfully reports on STX:Sxph interactions. Comparison of the NpSxph and RcSpxh STX poses shows essentially identical interactions with the tricyclic bis-guanidium core and reveals that the carbamate is able to occupy the pocket opened by the Y→I variant (FIG. 6C), as observed in RcSxph Y558I (FIG. 3B). This change, together with the more rigid nature of the NpSxph STX binding pocket likely contributes to the higher affinity of NpSxph for STX relative to RcSxph (Table 1). Taken together, the various structures of Sxph:STX complexes show how subtle changes, particularly at the Tyr558 position can influence STX binding and underscore that knowledge of the STX binding code can be used to tune the STX binding properties of different Sxphs.

### 2. Discussion

Our biochemical and structural characterization of a set of RcSxph mutants and Sxphs from diverse anurans reveals a conserved STX recognition code centered around six amino acid residues comprising two triads. One triad engages the STX bis-guanidinium core using carboxylate groups that coordinate each ring (RcSxph Glu540 and Asp794) and an aromatic residue that makes a cation-p interaction (RcSxph Phe784) with the STX concave face. This recognition motif is shared with Na_{V}s, the primary target of STX in PSP (Thomas-Tran and DuBois, Shen et al. 2018, Shen et al. 2019) (FIG. 2C and D) and showcases a remarkably convergent STX recognition strategy. The second amino acid triad (RcSxph residues Tyr558, Phe561, and Pro727) largely interacts with the carbamate moiety and contains a site, Tyr558 and its supporting residue Ile782, where amino acid changes, including those found in some Anuran Sxphs (FIG. 5), enhance STX binding. Structural studies of RcSxph mutants and the High Himalaya Frog NpSxph show that STX-affinity enhancing changes in this region of the binding site act by reducing the degree of conformational change associated with STX binding (FIGS. 3 and 6 C-D). These findings reveal one strategy for creating high affinity STX binding sites. Importantly, enhancing the affinity of Sxph for STX through changes at either site increases the capacity of RcSxph to rescue Na_{V}s from STX block (FIG. 4) and demonstrates that an understanding of the STX recognition code enables rational modification of Sxph binding properties. Thus, exploiting the information in the STX recognition code defined here should enable design of Sxphs as STX sensors or agents for treating STX poisoning.

Although STX binding activity has been reported in a variety of diverse invertebrates (Llewellyn et al.) and vertebrates (Llewellyn et al., Tanaka et al.), only two types of STX binding proteins have been identified and validated, Sxphs from frogs (Mahar et al., Abderemane-Ali et al.) and the STX and TTX binding proteins from pufferfish (Yotsu et al. 2001, Yotsu et al. 2010). Our discovery of a set of ten new Sxphs that bind STX with high affinity (FIGS. 5, S7, and S8) represents a substantial expansion of the Sxph family and reveals diverse natural variation of residues that are important for STX binding (FIG. 5B). Most notably, E540D, a change that compromises STX binding in RcSxph by ˜14-fold, occurs in five of the newly identified Sxphs. Nevertheless, functional studies show that RiSxph, which bears an Asp at this site, binds STX more strongly than RcSxph (Table 2). Hence, the natural variations at other RiSxph STX binding pocket sites must provide compensatory interactions to maintain a high STX binding affinity. Understanding how such variations impact STX engagement or influence the capacity of these proteins to discriminate among STX congeners (Llewellyn et al.) remain important unanswered questions. The striking abundance of Sxphs in diverse amphibians, representing linages separated by ˜140 million years (Feng et al.), that are not known to carry STX raises intriguing questions regarding the selective pressures that have caused these disparate amphibians to maintain this STX binding protein.

Besides the conserved STX binding site, all of the amphibian Sxphs possess a set of Thy1 domains similar to those in RcSxph that are known to act as protease inhibitors (Lenarčič et al.). The discovery of Sxphs in diverse Anurans shows that these domains are a common feature of the Sxph family and occur in varied numbers. The expansion of this insertion into the Sxph scaffold of 15-16 Thy1 domains in toad Sxphs is particularly striking. Structural comparisons between RcSxph and NpSxph, both of which have two Thy1 domains (FIG. 5A), show that these domains can adopt different positions with respect to the shared transferrin core (FIG. S10F). Whether these Thy1 domains are important for Sxph-mediated toxin resistance mechanisms (Mahar et al., Abderemane-Ali et al.) or serve some other function and whether the diversity of Thy1 repeats impacts function remains unknown. Our definition of Sxph STX binding code, which provides a guide for deciphering variation in the Sxph STX binding site (FIG. 5B), and high variability in Thy1 repeats among Anuran Sxphs should provide a guide for finding other Sxphs within this widespread and diverse family of amphibians family.

STX interacts with a variety of target proteins including select Nav isoforms (Duran-Riveroll and Cembella) and other channels (Su et al., Wang et al.), diverse soluble STX binding proteins (Mahar et al., Yen et al., Yotsu et al. 2001, Yotsu et al. 2010, Takati et al., Lin et al.), and some enzymes (Llewellyn, Lukowski et al. 2019, Lukowski et al. 2020). The identification of the Sxph STX recognition code together with the substantial expansion of the Sxph family provide a foundation for developing a deeper understanding of the factors that enable proteins to bind STX with high affinity. Exploration of such factors should be facilitated by the TF, FP, and ITC assays established here that enable Sxph:STX interactions to be probed using a range of samples quantities (TF: 600 ng STX, 25 μg Sxph; FP: 1 ng F-STX, 3 μg Sxph; ITC: 5 μg STX, 300 μg Sxph). In cases of limited samples, such as difficult to obtain STX congeners, the excellent agreement among the assays should provide a reliable basis for interpretation of binding properties. These assays, together with delineation of the Sxph STX binding code, together with the expansion of the Sxph family here provide a framework for understanding the lethal effects of this potent neurotoxin and ‘toxin sponge’ STX resistance mechanisms (Mahar et al., Abderemane-Ali et al.). This knowledge and may enable the design of novel PSP toxin sensors and agents that could mitigate STX intoxication.

### 3. Materials and Methods

### Expression and Purification of Sxphs and Mutants

R. catesbeiana Sxph (RcSxph) and mutants were expressed using a previously described RcSxph baculovirus expression system in which RcSxph carries in series, a C-terminal 3C protease cleavage site, green fluorescent protein (GFP), and a Hisio tag (Yen et al.). The gene encoding Nanorana parkeri Sxph (NpSxph) including its N-terminal secretory sequence (GenBank: XM_018555331.1) was synthesized and subcloned into a pFastBac1 vector using NotI and XhoI restriction enzymes by GenScript and bears the same C-terminal tags as RcSxph. RcSxph and NpSxph mutants were generated using the QuikChange site-directed mutagenesis kit (Stratagene). All constructs were sequenced completely. RcSxph, RcSxph mutants, NpSxph and NpSxph I559Y were expressed in Spodoptera frugiperda (Sf9) cells using a baculovirus expression system as described previously for RcSxph (Yen et al.) and purified using a final size exclusion chromatography (SEC) run in 150 mM NaCl, 10 mM HEPES, pH 7.4. Protein concentrations were determined by measuring UV absorbance at 280 nm using the following extinction coefficients calculated using the ExPASY server (https://web.expasy.org/protparam/): RcSxph Y558 mutants, 94,875 M⁻¹ cm⁻¹; RcSxph F784C 96,490 M⁻¹ cm⁻¹; RcSxph F784Y 97,855 M⁻¹ cm⁻¹, RcSxph and all other RcSxph mutants 96,365 M⁻¹ cm⁻¹; NpSxph 108,980 M⁻¹ cm⁻¹; and NpSxph I559Y 110,470 M⁻¹ cm⁻¹.

### Thermofluor (TF) Assay of Toxin Binding

Thermofluor assays for STX and TTX binding were developed as outlined (Huynh and Partch). TTX was purchased from Abcam (Catalog #ab120054). 20 μL samples containing 1.1 μM RcSxph, NpSxph, or mutants thereof, 5×SYPRO Orange dye (Sigma-Aldrich, S5692, stock concentration 5000×), 0-20 μM STX or TTX, 150 mM NaCl, 10 mM HEPES, pH 7.4 were set up in 96-well PCR plates (Bio-Rad), sealed with a microseal B adhesive sealer (Bio-Rad) and centrifuged (1 min, 230×g) prior to thermal denaturation. The real-time measurement of fluorescence using the HEX channel (excitation 515-535 nm, emission 560-580 nm) was performed in CFX Connect Thermal Cycler (Bio-Rad). Samples were heated from 25° C. to 95° C. at 0.2° C. min⁻¹. Melting temperature (Tm) was calculated by fitting the denaturation curves using a Boltzmann sigmoidal function and GraphPad Prism: F=Fₘᵢₙ+(Fₘₐₓ−Fₘᵢₙ)/(1+exp((Tm−T)/C)), where F is the fluorescence intensity at temperature T, Fₘᵢₙ and Fₘₐₓ are the fluorescence intensities before and after the denaturation transition, respectively, Tm is the midpoint temperature of the thermal unfolding transition, and C is the slope at Tm (Huynh and Partch). □Tm=Tm_{Sxph+20 μM toxin}−Tm_{Sxph}.

### Fluorescence Polarization Assay

Fluorescence polarization assays were performed as described (Rossi and Taylor). 100 μL samples containing 1 nM fluorescein labeled STX (F-STX), 150 mM NaCl, 10 mM HEPES, pH 7.4, and Sxph variants at the following concentration ranges (RcSxph and RcSxph T563A, I782A, F784Y, D785N, Q787A, Q787E, K789A, and Y795A, 0-75 nM; RcSxph Y558 Å and I782A/Y558A, 0-24 nM; RcSxph Y558I, 0-37.5 nM; RcSxph Y558F, 0-100 nM; RcSxph I782F 0-150 nM; RcSxph F561A, 0-300 nM; RcSxph F784L, 0-500 nM; RcSxph E540D, P727A, and D785A, 0-600 nM; RcSxph F784A, 0-4.8 μM; RcSxph E540 Å and F784C, 0-10 μM; RcSxph D794 Å and D794E, 0-12.5 μM; RcSxph F784S, 0-17 μM; RcSxph D794N, 0-20 μM; RcSxph E540Q, 0-25 μM; NpSxph and NpSxph I559Y, 0-75 nM) were prepared in 96-well black flat-bottomed polystyrene microplates (Greiner Bio-One) and sealed with an aluminum foil sealing film (AlumaSeal II), and incubated at room temperature for 0.5 h before measurement. Measurements were performed at 25° C. on a Synergy H1 microplate reader (BioTek) using the polarization filter setting (excitation 485 nm, emission 528 nm). Binding curves for representative high affinity (RcSxph, NpSxph, and RcSxph-Y558I) and low affinity (RcSxph-E540D) proteins were compared at 0.5 h, 1.5 h, 4.5 h, and 24 h, post mixing and indicated that equilibrium was reached by 0.5 h for all samples. The dissociation constants were calculated using GraphPad Prism by fitting fluorescence millipolarization (mP=P·10⁻³, where P is polarization) as a function of Sxph concentration using the equation: P={(P_{bound}−P_{free}) [Sxph]/(K_{d}+[Sxph])}+P_{free}, where P is the polarization measured at a given Sxph concentration, Pfᵣₑₑ is the polarization of Sxph in the absence of F-STX, and P_{bound} is the maximum polarization of Sxph bound by F-STX (Rossi and Taylor, Hansen et al.).

### Isothermal Titration Calorimetry (ITC)

ITC measurements were performed at 25° C. using a MicroCal PEAQ-ITC calorimeter (Malvern Panalytical). RcSxph, RcSxph mutants, NpSxph, and NpSxph I559Y were purified using a final size exclusion chromatography step in 150 mM NaCl, 10 mM HEPES, pH 7.4. 1 mM STX stock solution was prepared by dissolving STX powder in MilliQ water. This STX stock was diluted with the SEC buffer to prepare 100 μM or 300 μM STX solutions having a final buffer composition of 135 mM NaCl, 9 mM HEPES, pH 7.4. To match buffers between the Sxph and STX solutions, the purified protein samples were diluted with MilliQ water to reach a buffer concentration of 135 mM NaCl, 9 mM HEPES, pH 7.4. (30 μM for RcSxph D794E; 10 μM for RcSxph, other RcSxph mutants, NpSxph, and NpSxph I559Y) Protein samples were filtered through a 0.22 μm spin filter (Millipore) before loading into the sample cell and titrated with STX (300 μM STX for RcSxph D794 and 100 μM STX for RcSxph, other RcSxph mutants, NpSxph, and NpSxph I559Y) using a schedule of 0.4 μL titrant injection followed by 35 injections of 1 μL for the strong binders (RcSxph, RcSxph Y558I, RcSxph Y558A, RcSxph F561A, NpSxph, and NpSxph I559Y) and a schedule of 0.4 μL titrant injection followed by 18 injections of 2 μL for the weak binders (RcSxph P727A, RcSxph E540D, and RcSxph D794E). The calorimetric experiment settings were: reference power, 5 μcal/s; spacing between injections, 150 s; stir speed 750 rpm; and feedback mode, high. Data were analyzed using MicroCal PEAQ-ITC Analysis Software (Malvern Panalytical) using a single binding site model. The heat of dilution from titrations of 100 μM STX in 135 mM NaCl, 9 mM HEPES, pH 7.4 into 135 mM NaCl, 9 mM HEPES, pH 7.4 was subtracted from each experiment to correct the baseline.

### Crystallization, Structure Determination, and Refinement

RcSxph mutants were crystallized at 4° C. as previously described for RcSxph (Yen et al.). Briefly, purified protein was exchanged into a buffer of 10 mM NaCl, 10 mM HEPES, pH 7.4 and concentrated to 65 mg ml⁻¹ using a 50-kDa cutoff Amicon Ultra centrifugal filter unit (Millipore). Crystallization was set up by hanging drop vapor diffusion using a 24-well VDX plate with sealant (Hampton Research) using 3 μL drops having a 2:1 (v:v) ratio of protein:precipitant. For co-crystallization with STX, STX and the target RcSxph mutants were mixed in a molar ratio of 1.1:1 STX:Sxph and incubated on ice for 1 hour before setting up crystallization. RcSxph-Y558I and RcSxph-Y558I:STX were crystallized from solutions containing 27% 2-methyl-2,4-pentanediol, 5% PEG 8000, 0.08-0.2 M sodium cacodylate, pH 6.5. RcSxph-Y558 Å and RcSxph-Y558A:STX were crystallized from solutions containing 33% 2-methyl-2,4-pentanediol, 5% PEG 8000, 0.08-0.2 M sodium cacodylate, pH 6.5. To obtain crystals of the RcSxph:F-STX complex, RcSxph was crystallized from solutions containing 33% 2methyl-2,4-pentanediol, 5% PEG 8000, 0.11-0.2 M sodium cacodylate, pH 6.5 and then soaked with F-STX (final concentration, 1 mM) for 5 hours before freezing.

For NpSxph crystallization, protein was purified as described for RcSxph, except that the final size exclusion chromatography was done using 30 mM NaCl, 10 mM HEPES, pH 7.4. Protein was concentrated to 30-40 mg ml⁻¹ using a 50-kDa cutoff Amicon Ultra centrifugal filter unit (Millipore). NpSxph crystals were obtained by hanging drop vapor diffusion at 4° C. using 1:1 v/v ratio of protein and precipitant. NpSxph crystals were obtained from 400 nl drops set with Mosquito crystal (Sptlabtech) using 20-25% (v/v) PEG 400, 4-5% (w/v) PGA-LM, 100-200 mM sodium acetate, pH 5.0. For STX co-crystallization, NpSxph and STX (5 mM stock solution prepared in MilliQ water) were mixed in a molar ratio of 1.2:1 STX:NpSxph and incubated on ice for 1 hour before setting up the crystallization trays. Crystals of the STX:NpSxph were grown in the same crystallization solution as NpSxph. NpSxph and NpSxph:STX crystals were harvested and flash-frozen in liquid nitrogen without additional cryoprotectant.

X-ray datasets for RcSxph mutants, RcSxph mutant:STX complexes, RcSxph: F-STX, NpSxph, and NpSxph:STX were collected at 100K at the Advanced Photon Source (APS) beamline 23 ID B of Argonne National Laboratory (Lemont, IL), processed with XDS (Kabsch, 2010) and scaled and merged with Aimless (Evans and Murshudov). RcSxph structures were determined by molecular replacement of RcSxph chain B from (PDB: 6O0F) using Phaser from PHENIX (Adams et al.). The resulting electron density map was thereafter improved by rigid body refinement using phenix.refine. The electron density map obtained from rigid body refinement was manually checked and rebuilt in COOT (Emsley and Cowtan) and subsequent refinement was performed using phenix.refine.

The NpSxph structure was solved by molecular replacement using the MoRDa pipeline implemented in the Auto-Rikshaw, automated crystal structure determination platform (Panjikar et al.). The scaled X-ray data and amino-acid sequence of NpSxph were provided as inputs. The molecular replacement search model was identified using the MoRDa domain database derived from the Protein Data Bank (PDB). The MR solution was refined with REFMAC5 (N. Collaborative Computational Project), density modification was performed using PIRATE (Cowtan 2000, Winn et al.), and was followed by the automated model building in BUCCANEER (Cowtan 2006, Cowtan 2008). The partial model was further refined using REFMAC5 and phenix.refine. Dual fragment phasing was performed using OASIS-2006 (Winn et al.) based on the automatically refined model, and the resulting phases were further improved in PIRATE. The next round of model building was continued in ARP/wARP (Morris et al.) and the resulting structure was refined in REFMAC5. The final model generated in Auto-Rikshaw (720 out of 825 residues built, and 625 residues automatically docked) was further used as a MR search model in Phaser from PHENIX (Adams et al.). The quality of the electron density maps allowed an unambiguous assignment of most of the amino acid residues with the exception of the loop regions and the C2 subdomain showing poor electron density. The apo-NpSxph structure was completed by manual model building in COOT (Emsley and Cowtan) and multiple rounds of refinement in phenix.refine. The NpSxph:STX: structure was solved by molecular replacement using the NpSxph structure as a search model in Phaser from PHENIX (Adams et al.). After multiple cycles of manual model rebuilding in COOT (Emsley and Cowtan), iterative refinement was performed using phenix.refine. The quality of all models was assessed using MolProbity (Williams et al.) and refinement statistics.

RNA sequencing of O. sylvatica, D. tinctorius, R. imitator, E. tricolor, A. femoralis, and M. aurantiaca Sxphs

Nearly all poison frog species were bred in the O'Connell Lab or purchased from the pet trade (Josh's Frogs) except for O. sylvatica, which was field collected as described in (McGugan et al.). De novo transcriptomes for O. sylvatica, D. tinctorius, R. imitator, E. tricolor, A. femoralis, and M. aurantiaca were constructed using different tissue combinations depending on the species. RNA extraction from tissues was performed using TRIzol™ Reagent (Thermo Fisher Scientific). Poly-adenylated RNA was isolated using the NEXTflex PolyA Bead kit (Bioo Scientific, Austin, USA) following manufacturer's instructions. RNA quality and lack of ribosomal RNA was confirmed using an Agilent 2100 Bioanalyzer or Tapestation (Agilent Technologies, Santa Clara, USA). Each RNA sequencing library was prepared using the NEXTflex Rapid RNAseq kit (Bioo Scientific). Libraries were quantified with quantitative PCR (NEBnext Library quantification kit, New England Biolabs, Ipswich, USA) and an Agilent Bioanalyzer High Sensitivity DNA chip, according to manufacturer's instructions. All libraries were pooled at equimolar amounts and were sequenced on four lanes of an Illumina HiSeq 4000 machine to obtain 150 bp paired-end reads. De novo transcriptomes were assembled using Trinity and once assembled were used to create a BLAST nucleotide database using the BLAST+ command line utilities. The amino acid Sxph sequence of L. catesbeiana was used as a query to tBLASTN against the reference transcriptome databases. The Sxph sequence for O. sylvatica was lacking the 5′ and 3′ ends, whose sequence was obtained using RACE as described above. After obtaining a full-length sequence, the top BLAST hits from each poison frog transcriptome were manually inspected and aligned to the O. sylvatica nucleotide sequence to find full sequences with high similarity. Either a single Sxph sequence from each transcriptome was found to be the best match, or there were multiple transcripts that aligned well, in which case a consensus alignment was created. The largest ORF from each species sequence was translated to create an amino acid sequence for alignment. For the D. tinctorius, R. imitator, and A. femoralis sequences, regions covering the STX binding site and transferrin-related iron-binding sites were confirmed by PCR and sanger sequencing.

Identification of P. terribilis, R. Marina, B. Bufo, and B. gargarizans Sxphs

All P. terribilis frogs were captive bred in the O'Connell lab poison frog colony. All were sexually mature individuals housed in 18×18×18-inch glass terraria, brought up on a diet of Drosophila melanogaster without additional toxins. Frogs were euthanized according to the laboratory collection protocol detailed by Fischer et al. 2019 and tissues were stored in RNALater. Eye tissue was rinsed in PBS before being placed into the beadbug tubes (Sigma-Aldrich, Z763756) prefilled with 1 mL TRIzol (Thermo Fisher Scientific, 15596018) and then RNA was extracted following manufacturer instructions. RNA was reverse transcribed into cDNA following the protocol outlined in Invitrogen's SuperScript IV Control Reactions First-Strand cDNA Synthesis reaction (Pub. no. MAN0013442, 16 Rev. B). After reverse transcription, cDNA concentration was checked via NanoDrop (Thermo Scientific, ND-ONE-W), and then aliquoted and stored at −20° C. until used for PCR. Saxiphilin was amplified from cDNA from P. terribilis in 50 μL polymerase chain reactions following the New England Biolabs protocol for Phusion® High-Fidelity PCR Master Mix with HF Buffer (30) (included DMSO). Each reaction was performed with 1 μL of cDNA. PCR primers were designed based on a O. sylvatica saxiphilin cDNA sequence previously generated by the O'Connell lab. PCR products were cleaned up using the Thermo Scientific GeneJET Gel Extraction and DNA Cleanup Micro Kit (Catalog number K0832) dimer removal protocol, and then sent out for Sanger Sequencing via the GeneWiz “Premix” service. The segments from sequencing were aligned and assembled but found that the 5′ and 3′ ends of the Sxph sequence for P. terribilis were missing, thus the 5′ and 3′ end sequences were subsequently obtained using RACE. 5′ and 3′-RACE-Ready cDNA templates were synthesized using a SMARTer® RACE 5′/3′ Kit (Takara Bio, USA) and subsequently used to amplify 5′ and 3′ end sequences of P. terribilis Sxph using internal gene specific primers.

Initial Sxph sequence for R. marina was obtained from the genome by searching the draft Cane Toad genome (Edwards et al.) with tBLASTN using the L. catesbeiana Sxph amino acid sequence as a query. Matching segments from the genome were pieced together to produce an amino acid sequence, however, this sequence was missing part of the 3′ end. To obtain the 3′ residues, the nucleotide sequences from the genome were used to design primers for 3′ Rapid Amplification of cDNA Ends (RACE). One R. marina individual from a lab-housed colony was thus euthanized in accordance with UCSF IACUC protocol AN136799, and a portion of the liver was harvested for total RNA extraction using TRIzol™ Reagent (Thermo Fisher Scientific). Total RNA integrity was assessed on a denaturing formaldehyde agarose gel. 3′-RACE-Ready cDNA template was synthesized using a SMARTer® RACE 5′/3′ Kit (Takara Bio, USA) and subsequently used to amplify 3′ end sequences of R. marina Sxph using internal gene specific primers designed from R. marina genomic sequences. 3′ end sequences of R. marina Sxph were determined by gel extraction using QIAquick Gel Extraction Kit (QIAGEN) and verified by sanger sequencing.

Sequences for Bufo (Common Toad) and Bufo gargarizans (Asiatic toad) Sxphs were identified as sequence searches (tBLASTN) using the RmSph sequence as a query.

### Two-Electrode Voltage Clamp Electrophysiology

Two-electrode voltage-clamp (TEVC) recordings were performed on defolliculated stage V-VI Xenopus laevis oocytes harvested under UCSF-IACUC protocol AN178461. Capped mRNA for P. terribilis (Pt) Na_{V}14 (GenBank: MZ545381.1) expressed in a pCDNA3.1 vector (Abderemane-Ali et al.) was made using the mMACHINE™ T7 Transcription Kit (Invitrogen). Xenopus oocytes were injected with 3-6 ng of PtNa_{V}14 and TEVC experiments were performed 1-2 days post-injection. Data were acquired using a GeneClamp 500B amplifier (MDS Analytical Technologies) controlled by pClamp software (Molecular Devices), and digitized at 1 kHz using Digidata 1332 Å digitizer (MDS Analytical Technologies).

Oocytes were impaled with borosilicate recording microelectrodes (0.3-3.0 MΩ resistance) backfilled with 3 M KCl. Sodium currents were recorded using a bath solution containing the following, in millimolar: 96, NaCl; 1, CaCl₂; 1, MgCl₂; 2, KCl; and 5, HEPES (pH 7.5 with NaOH), supplemented with antibiotics (50 μg ml⁻¹ gentamycin, 100 IU ml⁻¹ penicillin and 100 μg ml⁻¹ streptomycin) and 2.5 mM sodium pyruvate.

Sxph responses were measured using Sxph or Sxph mutants purified as described above. Following recording of channel behavior in the absence of toxin, 100 nM STX was applied to achieve ˜90% block. Sxph was then added directly to a 1 mL recording chamber containing the toxin to the desired concentration. For all [Sxph]:[STX] ratios, the concentration of the stock Sxph solution added to the chamber was adjusted so that the volume of the added Sxph solution was less than 1% of the total volume of the recording chamber.

All toxin effects were assessed with 60-ms depolarization steps from −120 to 0 mV with a holding potential of −120 mV and a sweep-to-sweep duration of 10 s.

Recordings were conducted at room temperature (23±2° C.). Leak currents were subtracted using a P/4 protocol during data acquisition. Data Analysis was performed using Clampfit 10.6 (Axon Instruments) and SigmaPlot (Systat Software).

### F-STX Synthesis

All reagents were obtained commercially unless otherwise noted. N,N-Dimethylformamide (DMF) was passed through two columns of activated alumina prior to use. High-performance liquid chromatography-grade CH₃CN and H₂O were obtained from commercial suppliers. Semi-preparative high-performance liquid chromatography (HPLC) was performed on a Varian ProStar model 210. A high-resolution mass spectrum of F-STX was obtained from the Vincent Coates Foundation Mass Spectrometry Laboratory at Stanford University. The sample was analyzed with HESI-MS by direct injection onto Waters Acquity UPLC and a Thermo Fisher Orbitrap Exploris™ 240 mass spectrometer scanning m/z 100-1000. F-STX was quantified by ¹H NMR spectroscopy on a Varian Inova 600 MHz NMR instrument using distilled DMF as an internal standard. A relaxation delay (dl) of 20 s and an acquisition time (at) of 10 s were used for spectral acquisition. The concentration of F-STX was determined by integration of ¹H signals corresponding to F-STX and a fixed concentration of the DMF standard.

To an ice-cold solution of saxitoxin-N21-hexylamine (1.4 μmol) in 140 μL of pH 9.5 aqueous bicarbonate buffer (0.2 M aqueous NaHCO₃, adjusted to pH 9.5 with 1 M aqueous NaOH) was added a solution of fluorescein NHS-ester, 6-isomer (2.0 mg, 4.2 mol, 3.0 equiv, Lumiprobe) in 140 μL of DMSO. The reaction flask was stoppered, wrapped in foil, and placed in a sonication bath for 30 s. The reaction mixture was then stirred at room temperature for 4 h. Following this time, the reaction was quenched by the addition of 0.3 mL of 1% aqueous CF₃CO₂H. The reaction mixture was diluted with 1.1 mL of 10 mM aqueous CF₃CO₂H and 0.3 mL of DMSO and filtered through a VWR 0.22 μm PTFE filter. The product was purified by reverse-phase HPLC (Silicycle SiliaChrom dt C18, 5 μm, 10×250 mm column, eluting with a gradient flow of 10→40% CH₃CN in 10 mM aqueous CF₃CO₂H over 40 min, 214 nm UV detection). At a flow rate of 4 mL/min, F-STX had retention time of 31.00 min and was isolated as a dark yellow powder following lyophilization (1.08 μmol, 77%, ¹H NMR quantitation).

¹H NMR (600 MHz, D₂O) δ 8.05 (d, J=8.1 Hz, 1H), 7.94 (d, J=8.9 Hz, 1H), 7.48 (s, 1H), 6.95 (d, J=9.0 Hz, 2H), 6.79 (s, 2H), 6.67 (dt, J=9.1, 2.2 Hz, 2H), 4.60 (d, J=1.2 Hz, 1H), 4.09-4.05 (m, 1H), 3.89 (dd, J=11.6, 5.2 Hz, 1H), 3.70 (dt, J=10.1, 5.5 Hz, 1H), 3.64 (dd, J=8.7, 5.4 Hz, 1H), 3.47-3.42 (m, 1H), 3.27 (t, J=6.6 Hz, 2H), 2.97-2.89 (m, 2H), 2.36-2.33 (m 1H), 2.30-2.24 (m, 1H), 1.48-1.45 (m, 2H), 1.32-1.29 (m, 2H), 1.25-1.21 (m, 4H) ppm. HRMS (ESI⁺) calcd for C₃₇H₄₁N₈O₁₀, 757.2940; found 757.2918 (M⁺).

### Example 2: Protocol for Testing In Vivo Efficacy of Sxphs as Toxin Antidotes

Saxitoxin (STX) is one of the most lethal natural paralytic neurotoxins due to its ability to stop electrical signals in nerves by inhibiting the action of proteins known as voltage-gated sodium channels (Na_{V}s). Because of its extreme potency and lethality, STX is classified as a chemical weapon. STX and related toxins are produced by bacteria and plankton associated with oceanic red tides and cause paralytic shellfish poisoning (PSP). There are no antidotes that can be administered to mitigate STX poisoning. Remarkably, some animals, particularly select frog species, are naturally resistant to STX poisoning. How these creatures evade the lethal effects of STX remains unknown.

A leading candidate is a class of soluble high-affinity STX binding proteins known as saxiphilins (Sxphs). These and related proteins are thought to act as ‘toxin sponges’ that sequester and my help eliminate STX, thereby protecting the frog nervous system from STX inhibition. The experiments outlined below are directed at testing whether high-affinity STX binding proteins, Sxphs, can be used as countermeasures against STX poisoning. We pursue two approaches that will test the ability of Sxphs and related proteins to reverse STX poisoning in mouse models.

Individual trials will use 6-10 mice for each condition tested. These values are based on previous studies used to test anti-STX and anti-TTX antibodies (Davio, Fukiya and Matsumura). Because there are reports of differential effects on male vs. female mice, all trials will include equal numbers of males and females in each test condition.

Initial studies will need 60 animals (6 different conditions, 10 mice each; STX, Sham, Sxph, Sxph/STX, TTX, TTX/Sxph). We expect to need to test various rations of Sxph/STX as well as Sxph mutants having enhanced or reduced STX binding capacity. For those experiments, we will have to have controls (STX, Sxph, Sham) that will each require 10 mice. These numbers are based on similar published experiments testing the efficacy of anti-STX antisera (Davio); 10 mice per condition) and anti-TTX IgG ((Fukiya and Matsumura); 12 mice per condition).

Our objective is to test whether saxiphilins (Sxphs) or similar high affinity STX binding proteins can be used as an antidote for STX poisoning. Using in vitro models, we have recently shown that bullfrog (Rana castesbeiana) Sxph (RcSxph) can effectively rescue voltage-gated sodium channels from STX block (Abderemane-Ali et al.). This in vitro demonstration is a key proof-of-concept that underpins the proposed animal studies. Even though we can show that Sxph application to a cell expressing a voltage-gated sodium channel can rescue the channel from STX block, it is impossible to know if Sxph can do the same in a complex physiological setting. Hence, in order to test whether Sxphs can be developed as antidotes for STX poisoning, we have to use an animal system.

Mice (Mus musculus) are the best species for testing reversal of STX effects. Their sensitivity to STX poisoning is the bases for the gold-standard assay that is used to test shellfish for human consumption to ensure that the shellfish are not contaminated with STX (AOAC). Prior work testing the efficacy of rabbit STX antisera (Davio, Kaufman et al.) and a donkey anti-STX antibody (Benton et al.) conducted experiments using a mouse model system. As these are the only studies in the literature for which we have a comparison, we think that the mouse model is the best system for the proposed experiments.

These experiments are designed to test whether saxiphilins, or other saxitoxin binding molecules, can be used as medical countermeasures against saxitoxin poisoning. Currently, no such treatments exist.

This protocol follows the basic i.p. injection protocol outlined by Munday et al., Based on Munday et al., 30 nmoles/Kg of STX delivered i.p. is sufficient to achieve 100% lethality within 10 minutes. This will be the initial quantity of STX used in our experiments. Based on Finch et al., 70 nM/Kg of TTX delivered i.p. is equivalent to 30 nmoles/Kg STX. This will be the initial quantity of TTX used in our experiments. This is an important control as Sxph does not bind TTX, and TTX and STX work by similar mechanisms of action.
1) STX, Sxph, TTX, Sxph:STX, or Sxph:TTX mixtures will be prepared in phospho-buffered saline (150 mM NaCl, 10 mM P04, pH 7.4).
2) Mice will be weighed before treatment.
3) Mice will be injected IP with 30 nmoles/Kg STX, Sxph, or STX:Sxph mixtures. Initial experiments will aim for 1:1 or 1:2 molar rations of STX:Sxph as these are sufficient to reverse the effects of STX on sodium channels based on our recent work (Abderemane-Ali et al.).

Solutions will be prepared to deliver a 100 μl i.p. injection per animal using a 25-27G needle. Test solutions will be delivered IP as follows:
i) Mice will be restrained manually with the body tilted downward, and the head of the animal tilted back.
ii) Insert needle (25-27G needle size) with bevel facing “up” into the lower right quadrant of the abdomen towards the head at a 30-40° angle to horizontal. Insert needle to the depth in which the entire bevel is within the abdominal cavity.
iii) Deliver solution after aspiration. Only one injection will be delivered per mouse. No pregnant females will be used.
iv) Animals will be monitored following injection to note symptoms, time of onset, and time of death if it occurs. Based on prior work, Munday et al., we expect to see the following symptoms: lethargy, rapid abdominal breathing, immobility, irregular respiration, paralysis, cynanosis. To reduce pain and distress, if any animals having only the toxin administered are experiencing abdominal breathing, they will immediately be euthanized per section N because we can anticipate this animal will die very soon. Animals administered the possible antidote will need to be monitored longer so that we can detect if and when the antidote is able to reverse the effects of the toxin (see below).
v) Data of time to death will be plotted to assess efficacy of Sxph, Sxph/Toxin ratios, and Sxph variant function.

Based on the above references, we expect the animals to survive only ˜10 minutes in the absence of the toxin binding proteins. For animals that have only had the toxin administered, they will be euthanized at 10 minutes if the animals have not died by that point.

Animals receiving control injections (buffer only, toxin binding protein only) will be monitored for the same period. Provided these animals are not showing signs of distress, we will continue the monitoring to 1 hour in order to gauge the effectiveness of the antidote. These animals will be euthanized at the 1 hour time point.

### REFERENCES

Abderemane-Ali F. et al., Evidence that toxin resistance in poison birds and frogs is not rooted in sodium channel mutations and may rely on “toxin sponge” proteins. J Gen Physiol 153, (2021).
Adams, P D et al., PHENIX: a comprehensive Python-based system for macromolecular structure solution. Acta Crystallogr D Biol Crystallogr 66, 213-221 (2010).
Anderson, D M et al., Evidence for massive and recurrent toxic blooms of Alexandrium catenella in the Alaskan Arctic. Proc Natl Acad Sci USA 118, (2021).
Andresen, BM, J. Du Bois, De novo synthesis of modified saxitoxins for sodium ion channel study. J Am Chem Soc 131, 12524-12525 (2009).
AOAC, in Official Methods of Analysis. (Arlington, V A, 1999), vol. 1.
Arrigoni, D. L. Minor, Jr., Global versus local mechanisms of temperature sensing in ion channels. Pflugers Arch 470, 733-744 (2018).
Becktel, WJ, J. A. Schellman, Protein stability curves. Biopolymers 26, 1859-1877 (1987).
Benton, BJ. V. R. Rivera, J. F. Hewetson, F. C. Chang, Reversal of saxitoxin-induced cardiorespiratory failure by a burro-raised alpha-STX antibody and oxygen therapy. Toxicol Appl Pharmacol 124, 39-51 (1994).
Caty, S N, et al., Molecular physiology of chemical defenses in a poison frog. J Exp Biol 222, (2019).
Clackson, T., J. A. Wells, A hot spot of binding energy in a hormone-receptor interface. Science 267, 383-386. (1995).
Cowtan, K, General quadratic functions in real and reciprocal space and their application to likelihood phasing. Acta Crystallogr D Biol Crystallogr 56, 1612-1621 (2000).
Cowtan, K, The Buccaneer software for automated model building. 1. Tracing protein chains. Acta Crystallogr D Biol Crystallogr 62, 1002-1011 (2006).
Cowtan, K, Fitting molecular fragments into electron density. Acta Crystallogr D Biol Crystallogr 64, 83-89 (2008).
Davio, SR Neutralization of saxitoxin by anti-saxitoxin rabbit serum. Toxicon 23, 669-675 (1985).
Doyle, DD, M. Wong, J. Tanaka, L. Barr, Saxitoxin binding sites in frog-myocardial cytosol. Science 215, 1117-1119 (1982).
Duran-Riveroll, LM, A. D. Cembella, Guanidinium Toxins and Their Interactions with Voltage-Gated Sodium Ion Channels. Mar Drugs 15, (2017).
Edwards, R J, et al., Draft genome assembly of the invasive cane toad, Rhinella marina. Gigascience 7, (2018).
Emsley, P, K. Cowtan, Coot: model-building tools for molecular graphics. Acta Crystallogr D Biol Crystallogr 60, 2126-2132 (2004).
Evans, PR, G. N. Murshudov, How good are my data and what is the resolution? Acta Crystallogr D Biol Crystallogr 69, 1204-1214 (2013).
Feng, Y J, et al., Phylogenomics reveals rapid, simultaneous diversification of three major clades of Gondwanan frogs at the Cretaceous-Paleogene boundary. Proc Natl Acad Sci USA 114, E5864-E5870 (2017).
Finch, SC, M. J. Boundy, D. T. Harwood, The Acute Toxicity of Tetrodotoxin and Tetrodotoxin(-)Saxitoxin Mixtures to Mice by Various Routes of Administration. Toxins (Basel) 10, (2018).
Fukiya, S, K. Matsumura, Active and passive immunization for tetrodotoxin in mice. Toxicon 30, 1631-1634 (1992).
Hansen, S, et al., Regulation of the Escherichia coli HipBA toxin-antitoxin system by proteolysis. PLoS One 7, e39185 (2012).
Heinemann, S H, H. Terlau, K. Imoto, Molecular basis for pharmacological differences between brain and cardiac sodium channels. Pflugers Arch 422, 90-92 (1992).
Hille, B, Ion Channels ofExcitable Membranes. (Sinauer Associates, Inc., Sunderland, MA, ed. 3rd, 2001).
Huang, X, A. Aulabaugh, Application of Fluorescence Polarization in HTS Assays. Methods Mol Biol 1439, 115-130 (2016).
Huynh, K, C. L. Partch, Analysis of protein stability and ligand interactions by thermal shift assay. Curr Protoc Protein Sci 79, 28 29 21-14 (2015).
Infield, D T et al., Cation-pi Interactions and their Functional Roles in Membrane Proteins. J Mol Biol 433, 167035 (2021).
Kabsch, Xds. Acta Crystallogr D Biol Crystallogr 66, 125-132 (2010).
Kao, CY, F. A. Fuhrman, Differentiation of Actions of Tetrodotoxin and Saxitoxin. Toxicon 5, 25-34 (1967).
Kaufman, B., D. C. Wright, W. R. Ballou, D. Monheit, Protection against tetrodotoxin and saxitoxin intoxication by a cross-protective rabbit anti-tetrodotoxin antiserum. Toxicon 29, 581-587 (1991).
Lambert, H. Perri, P. J. Halbrooks, A. B. Mason, Evolution of the transferrin family: conservation of residues associated with iron and anion binding. Comp Biochem Physiol B Biochem MolBiol 142, 129-141 (2005).
Leffler, A, R. I. Herzog, S. D. Dib-Hajj, S. G. Waxman, T. R. Cummins, Pharmacological properties of neuronal TTX-resistant sodium channels and the role of a critical serine pore residue. Pflugers Arch 451, 454-463 (2005).
Lenarčič, B, et al., Saxiphilin, a saxitoxin-binding protein with two thyroglobulin type 1 domains, is an inhibitor of papain-like cysteine proteinases. J Biol Chem 275, 15572-15577 (2000).
Li, L. Llewellyn, E. Moczydlowski, Biochemical and Immunochemical Comparison of Saxiphilin and Transferrin, 2 Structurally Related Plasma-Proteins from Rana-Catesbeiana. Molecular Pharmacology 44, 742-748 (1993).
Lin, H, et al., Neutralizing effect of hemolymph from the shore crab, Thalamita crenata, on paralytic shellfish toxins. Toxicon 99, 51-57 (2015).
Llewellyn, LE, E. G. Moczydlowski, Characterization of saxitoxin binding to saxiphilin, a relative of the transferrin family that displays pH-dependent ligand binding. Biochemistry 33, 12312-12322 (1994).
Llewellyn, LE, P. M. Bell, E. G. Moczydlowski, Phylogenetic survey of soluble saxitoxin-binding activity in pursuit of the function and molecular evolution of saxiphilin, a relative of transferrin. Proc Biol Sci 264, 891-902 (1997).
Llewellyn, LE, Saxitoxin, a toxic marine natural product that targets a multitude of receptors. Nat Prod Rep 23, 200-222 (2006).
Lukowski, A L et al., Biocatalytic Detoxification of Paralytic Shellfish Toxins. ACS Chem Biol 14, 941-948 (2019).
Lukowski, A L, J. Liu, J. Bridwell-Rabb, A. R. H. Narayan, Structural basis for divergent C—H hydroxylation selectivity in two Rieske oxygenases. Nat Commun 11, 2991 (2020).
Mahar, J, G. L. Lukacs, Y. Li, S. Hall, E. Moczydlowski, Pharmacological and biochemical properties of saxiphilin, a soluble saxitoxin-binding protein from the bullfrog (Rana catesbeiana). Toxicon 29, 53-71 (1991).
McGugan, J R et al., Ant and Mite Diversity Drives Toxin Variation in the Little Devil Poison Frog. J Chem Ecol 42, 537-551 (2016).
Morabito, MA, E. Moczydlowski, Molecular cloning of bullfrog saxiphilin: a unique relative of the transferrin family that binds saxitoxin. Proc Natl Acad Sci USA 91, 2478-2482 (1994).
Morris, R J et al., Breaking good resolutions with ARP/wARP. J Synchrotron Radiat 11, 56-59 (2004).
Munday, R., K. Thomas, R. Gibbs, C. Murphy, M. A. Quilliam, Acute toxicities of saxitoxin, neosaxitoxin, decarbamoyl saxitoxin and gonyautoxins 1&4 and 2&3 to mice by various routes of administration. Toxicon 76, 77-83 (2013).
N. Collaborative Computational Project, The CCP4 suite: Programs for protein crystallography. Acta Crystallogr D Biol Crystallogr 50, 760-763. (1994).
Niesen, FH, H. Berglund, M. Vedadi, The use of differential scanning fluorimetry to detect ligand interactions that promote protein stability. Nat Protoc 2, 2212-2221 (2007).
O'Connell, L A et al., Rapid toxin sequestration modifies poison frog physiology. J Exp Biol 224, (2021).
Ondrus, A E, et al., Fluorescent saxitoxins for live cell imaging of single voltage-gated sodium ion channels beyond the optical diffraction limit. Chem Biol 19, 902-912 (2012).
Panjikar, S, V. Parthasarathy, V. S. Lamzin, M. S. Weiss, P. A. Tucker, Auto-rickshaw: an automated crystal structure determination platform as an efficient tool for the validation of an X-ray diffraction experiment. Acta Crystallogr D Biol Crystallogr 61, 449-457 (2005).
Prinzmetal, M., H. Sommer, C. D. Leake, The pharmacological action of “mussel poison”. J Pharmacol Exp Ther 46, 63-73 (1932).
Rossi, A M, C. W. Taylor, Analysis of protein-ligand interactions by fluorescence polarization. Nat Protoc 6, 365-387 (2011).
Satin, J. et al., A mutant of TTX-resistant cardiac sodium channels with TTX-sensitive properties. Science 256, 1202-1205 (1992). (Satin et al.)
Shen, H, et al., Structural basis for the modulation of voltage-gated sodium channels by animal toxins. Science 362, (2018).
Shen, H, D. Liu, K. Wu, J. Lei, N. Yan, Structures of human Na_{V}17 channel in complex with auxiliary subunits and animal toxins. Science 363, 1303-1308 (2019).
Sivilotti, L, K. Okuse, A. N. Akopian, S. Moss, J. N. Wood, A single serine residue confers tetrodotoxin insensitivity on the rat sensory-neuron-specific sodium channel SNS. FEBS Lett 409, 49-52 (1997).
Su, Z, M. Sheets, H. Ishida, F. Li, W. H. Barry, Saxitoxin blocks L-type ICa. J Pharmacol Exp Ther 308, 324-329 (2004).
Takati, N, D. Mountassif, H. Taleb, K. Lee, M. Blaghen, Purification and partial characterization of paralytic shellfish poison-binding protein from Acanthocardia tuberculatum. Toxicon 50, 311-321 (2007).
Tanaka, JC, D. D. Doyle, L. Barr, Sodium channels in vertebrate hearts. Three types of saxitoxin binding sites in heart. Biochim Biophys Acta 775, 203-214 (1984).
Thomas-Tran, R, J. Du Bois, Mutant cycle analysis with modified saxitoxins reveals specific interactions critical to attaining high-affinity inhibition of hNaV1.7. Proc Natl Acad Sci USA 113, 5856-5861 (2016).
Thottumkara, AP, W. H. Parsons, J. Du Bois, Saxitoxin. Angew Chem Int Ed Engl 53, 5760-5784 (2014).
Velazquez-Campoy, H. Ohtaka, A. Nezami, S. Muzammil, E. Freire, Isothermal titration calorimetry. Curr Protoc Cell Biol Chapter 17, Unit 17 18 (2004).
Wang, J, J. J. Salata, P. B. Bennett, Saxitoxin is a gating modifier of HERG K+ channels. J Gen Physiol 121, 583-598 (2003).
Wells, JA, Additivity of mutational effects in proteins. Biochemistry 29, 8509-8517 (1990).
Wells, JA, Systematic mutational analyses of protein-protein interfaces. Methods Enzymol 202, 390-411 (1991).
Wiese, M, P. M. D'Agostino, T. K. Mihali, M. C. Moffitt, B. A. Neilan, Neurotoxic alkaloids: saxitoxin and its analogs. Mar Drugs 8, 2185-2211 (2010).
Williams, C J et al., MolProbity: More and better reference data for improved all-atom structure validation. Protein Sci 27, 293-315 (2018).
Winn, M D et al., Overview of the CCP4 suite and current developments. Acta Crystallogr D Biol Crystallogr 67, 235-242 (2011).
Yen, TJ, M. Lolicato, R. Thomas-Tran, J. Du Bois, D. L. Minor, Jr., Structure of the Saxiphilin:saxitoxin (STX) complex reveals a convergent molecular recognition strategy for paralytic toxins. Sci Adv 5, (2019).
Yotsu-Yamashita, M et al., Purification, characterization, and cDNA cloning of a novel soluble saxitoxin and tetrodotoxin binding protein from plasma of the puffer fish, Fugu pardalis. Eur J Biochem 268, 5937-5946 (2001).
Yotsu-Yamashita, M, H. Yamaki, N. Okoshi, N. Araki, Distribution of homologous proteins to puffer fish saxitoxin and tetrodotoxin binding protein in the plasma of puffer fish and among the tissues of Fugu pardalis examined by Western blot analysis. Toxicon 55, 1119-1124 (2010).
Zhang, Q. Wu, M. Y. Berezin, Fluorescence anisotropy (polarization): from drug screening to precision medicine. Expert Opin Drug Discov 10, 1145-1161 (2015).

1. A system comprising:
(i) a solid support comprising at least one vessel, wherein the vessel comprises a solution comprising a first amino acid sequence that comprises at least about 70% sequence identity to X₁X₂X₃X₄X₅X₆X₇X₈X₉X₁₀X₁₁X₁₂X₁₃X₁₄; or
(ii) a solid support comprising a solid surface upon which the first amino acid sequence is absorbed, bound covalently or bound non-covalently, wherein the first amino acid sequence comprises at least about 70% sequence identity to X₁X₂X₃X₄X₅X₆X₇X₈X₉X₁₀X₁₁X₁₂X₁₃X₁₄;
wherein X₁=I, V, F, L
X₂=any amino acid
X₃=F
X₄=D
X₅=any amino acid
X₆=M, Q, I
X₇=any amino acid
X₈=R, K
X₉=any amino acid
X₁₀=any amino acid
X₁₁=any amino acid
X₁₂=any amino acid
X₁₃=D
X₁₄=Y, V

2. The system of claim 1, wherein the solid surface is a magnetic bead, a portion of a column, or a modified or unmodified plastic surface.

3. The system of claim 2, wherein the solid surface is a modified plastic surface comprising a linker bound to the first amino acid sequence.

4. (canceled)

5. The system of claim 3, wherein the linker is an antibody or antibody fragment immobilized to the surface of the solid surface comprising a complementary determinant region (CDR) specific to the first amino acid sequence.

6. The system of claim 1, wherein the amino acid sequence comprises at least 90% sequence identity to X₁X₂X₃X₄X₅X₆X₇X₈X₉X₁₀X₁₁X₁₂X₁₃X₁₄;
wherein X₁=I, V, F, L
X₂=any amino acid
X₃=F
X₄=D
X₅=any amino acid
X₆=M, Q, I
X₇=any amino acid
X₈=R, K
X₉=any amino acid
X₁₀=any amino acid
X₁₁=any amino acid
X₁₂=any amino acid
X₁₃=D
X₁₄=Y, V

7. The system of claim 1, wherein the first amino acid sequence comprises about 70% sequence identity to an amino acid comprising contiguous amino acids with Formula: X_{B}−[from about 53 to about 56 amino acids]−[[X₁X₂X₃X₄X₅X₆X₇X₈X₉X₁₀X₁₁X₁₂X₁₃X₁₄]; wherein
X_{B}=N or P
X₁=I, V, F, or L
X₂=any amino acid
X₃=F
X₄=D
X₅=any amino acid
X₆=M, Q, or I
X₇=any amino acid
X₈=R or K
X₉=any amino acid
X₁₀=any amino acid
X₁₁=any amino acid
X₁₂=any amino acid
X₁₃=D
X₁₄=Y or V

8. The system of claim 1, wherein the first amino acid sequence comprises at least about 75% sequence identity to an amino acid with contiguous amino acids of Formula III: [X₁ₐX₂ₐX₃ₐX₄ₐX₅ₐX₆ₐ]−[from about 161 to about 164 amino acids]−X_{B}−[from about 53 to about 56 amino acids]−[X₁X₂X₃X₄X₅X₆X₇X₈X₉X₁₀X₁₁X₁₂X₁₃X₁₄];
wherein
X₁ₐ=Y
X₂ₐ=any amino acid
X₃ₐ=an amino acid
X₄ₐ=F
X₅ₐ=any amino acid
X₆ₐ=S or G
X_{B}=N or P
X₁=I, V, F, or L
X₂=any amino acid
X₃=F
X₄=D
X₅=any amino acid
X₆=M, Q, or I
X₇=any amino acid
X₈=R or K
X₉=any amino acid
X₁₀=any amino acid
X₁₁=any amino acid
X₁₂=any amino acid
X₁₃=D
X₁₄=Y or V

9. The system of claim 1, wherein the first amino acid sequence comprises at least about 75% sequence identity to an amino acid with contiguous amino acids of Formula IV: [X_{A}]−[from about 15 to about 20 amino acids]−[X₁ₐX₂ₐX₃ₐX₄ₐX₅ₐX₆ₐ]−[from about 161 to about 164 amino acids]−X_{B}−[from about 53 to about 56 amino acids]−[X₁X₂X₃X₄X₅X₆X₇X₈X₉X₁₀X₁₁X₁₂X₁₃X₁₄]; wherein
X_{A}=D or E
X₁ₐ=Y
X₂ₐ=any amino acid
X₃ₐ=an amino acid
X₄ₐ=F
X₅ₐ=any amino acid
X₆ₐ=S or G
X_{B}=N or P
X₁=I, V, F, or L
X₂=any amino acid
X₃=F
X₄=D
X₅=any amino acid
X₆=M, Q, or I
X₇=any amino acid
X₈=R or K
X₉=any amino acid
X₁₀=any amino acid
X₁₁=any amino acid
X₁₂=any amino acid
X₁₃=D
X₁₄=Y or V

10. (canceled)

11. (canceled)

12. The system of claim 1, wherein the first amino acid is chosen from an amino acid sequence comprising a substitution mutation at amino acid number chosen from: 540, 558, 559, 561, 563, 727, 782, 784, 785, 787, 789, 794 and/or 795, in relation to such amino acid numbers identified in any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15.

13. (canceled)

14. (canceled)

15. (canceled)

16. (canceled)

17. The system of claim 1, wherein the amino acid is associated with a saxitoxin (STX) compound having a structure represented by a formula: 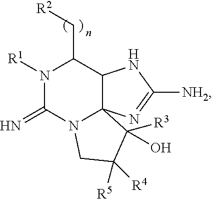
wherein n is selected from 0, 1, and 2;
wherein each of R¹ and R³ is independently selected from hydrogen and —OH;
wherein R² is selected from hydrogen, —OH, and —OC(O)R¹⁰;
wherein R¹⁰, when present, is selected from —NH₂, —CH₃, —NHSO₂H, —NHSO₃⁻, —NHSO₃H, and Ar¹;
wherein Ar¹, when present, is a C6 aryl substituted with 0, 1, 2, or 3 groups independently selected from —OH, —SO₃⁻, and —SO₃H; and
wherein each of R⁴ and R⁵ is independently selected from hydrogen, —OH, —OSO₃H, and —OSO₃⁻,
or a salt thereof.

18. The system of claim 1, wherein the amino acid is associated with a tetrodotoxin (TTX) compound having a structure represented by a formula selected from: 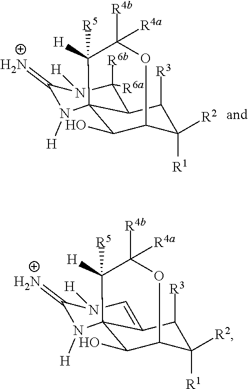
wherein R¹ is selected from hydrogen and —OH;
wherein R² is selected from —CH₃, —CH₂OH, and —CH(OH)₂;
wherein R³ is hydrogen and wherein each of R^{4a} and R^{4b} together comprise ═O,
or wherein R³ and R^{4a} together comprise —O— and wherein R^{4b} is —OH; and
wherein R⁵ is —OH and wherein each of R^{6a} and R^{6b} is independently selected from hydrogen and —OH,
or wherein R⁵ and R^{6b} together comprise —O— and wherein R^{6a} is hydrogen;
or a salt thereof.

18. (canceled)

19. Them system of claim 1, wherein the first amino acid further comprises comprising a fluorescent molecule, a dye, quantum dot, streptavidin, biotin, or an enzyme.

20. (canceled)

21. (canceled)

22. (canceled)

23. (canceled)

24. The system of claim 1, wherein the system is free of SEQ ID NO: 1, SEQ ID NO:2, SEQ ID NO:11, and SEQ ID NO:12.

25. (canceled)

26. (canceled)

27. (canceled)

28. (canceled)

29. (canceled)

30. (canceled)

31. (canceled)

32. (canceled)

33. A method of detecting the quantity or presence of a paralytic shellfish poisoning (PSP) toxin in a sample, the method comprising: (i) exposing the sample to a composition comprising an amino acid sequence having at least 90% sequence identity to least 90% sequence identity to X₁X₂X₃X₄X₅X₆X₇X₈X₉X₁₀X₁₁X₁₂X₁₃X₁₄;
wherein X₁=I, V, F, L
X₂=any amino acid
X₃=F
X₄=D
X₅=any amino acid
X₆=M, Q, I
X₇=any amino acid
X₈=R, K
X₉=any amino acid
X₁₀=any amino acid
X₁₁=any amino acid
X₁₂=any amino acid
X₁₃=D
X₁₄=Y, V

34. (canceled)

35. The method of claim 33 further comprising a step (ii) comprising measuring the amount of amino acid sequence bound to the PSP toxin present in the sample.

36. (canceled)

37. The method of claim 33, wherein the sample is from a mollusk.

38. (canceled)

39. The method of claim 33, wherein the amino acid sequence comprises a sequence chosen from Formula I, Formula II, Formula III or Formula IV.

40. (canceled)

41. (canceled)

42. (canceled)

43. The method of claim 35, wherein the step (ii) of measuring the amount of amino acid sequence bound to the PSP toxin comprises mixing a known amount of the amino acid sequence and the sample for a time period sufficient to associate the amino acid sequence to the saxitoxin or derivative thereof and measuring the association between the amino acid sequence and the saxitoxin or derivative thereof by one or more of: fluorescence, microscopy, chemiluminescence, elution, wavelength absorbance, or enzymatic cleavage.

44.-53. (canceled)

54. A method of assaying the toxicity of a subject comprising:
(i) exposing a sample from the subject to a composition comprising an amino acid sequence having at least 70% sequence identity to X₁X₂X₃X₄X₅X₆X₇X₈X₉X₁₀X₁₁X₁₂X₁₃X₁₄;
wherein X₁=I, V, F, L
X₂=any amino acid
X₃=F
X₄=D
X₅=any amino acid
X₆=M, Q, I
X₇=any amino acid
X₈=R, K
X₉=any amino acid
X₁₀=any amino acid
X₁₁=any amino acid
X₁₂=any amino acid
X₁₃=D
X₁₄=Y, V;
(ii) measuring an association between the amino acid sequence and a paralytic shellfish poisoning (PSP) toxin; and
(iii) determining that the subject comprises a toxic substance if the association between the amino acid sequence and the PSP toxin is higher than a threshold level of toxin; or determining that the subject is free of a toxic substance if the association between the amino acid sequence and the PSP toxin is lower than the threshold level of the toxin.

55. (canceled)

56. The method of claim 54, further comprising a step, after step (ii) but before step (iii), normalizing quantitative values obtained from measuring the association by subtracting or comparing the quantitative values obtained from the step of measuring to control values of association determined by a control.

57. The method of claim 54, wherein the sample is from a mollusk.

58. The method of claim 54, wherein the amino acid sequence comprises at least 90% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15 or a functional fragment thereof.

59. (canceled)

60. The method of, wherein the PSP toxin is a STX or TTX.

61. (canceled)

62. (canceled)

63. (canceled)

64. The method of claim 54, wherein the step of measuring further comprises:
(a) mixing a known amount of a control amino acid sequence with the sample for a time period sufficient for association of the amino acid sequence with the PSP toxin and measuring the association between the control amino acid sequence and the PSP toxin by one or a combination of fluorescence, microscopy, chemiluminescence, elution, wavelength absorbance, or enzymatic cleavage; and
(b) normalizing the association of the amino acid sequence to the PSP toxin the association of the control to the PSP toxin.

65. (canceled)

66. (canceled)

67. A kit comprising:
(a) the system of claim 1; and a first container comprising instructions.

68. (canceled)

69. (canceled)

70. A method of preparing a sample comprising or suspected of comprising a toxin comprising (i) exposing homogenate to a filter in the system of claim 1; and (ii) analyzing the homogenate for presence of a toxin.

71. (canceled)

72. The method of claim 70 wherein said filter comprises a saxiphilin or functional fragment thereof.

73. The method of claim 72, wherein said saxiphilin comprises a detection molecule covalently or non-covalently attached to said saxiphilin or functional fragment thereof.

74. The method of claim 72, wherein the step of analyzing comprises detecting whether a toxin bound to said saxiphilin or functional fragment thereof.
